SEQUENCE LISTING (9 sequences)

SEQ ID NO: 1 - DNA, 21 bp, synthetic construct
  source 1..21: mol_type = other DNA
cctggtgctccatgaggagac

SEQ ID NO: 2 - DNA, 22 bp, synthetic construct
  source 1..22: mol_type = other DNA
cagactctgaccttttgccagg

SEQ ID NO: 3 - DNA, 22 bp, synthetic construct
  source 1..22: mol_type = other DNA
tcttgtcgtcttcggaaatgtt

SEQ ID NO: 4 - DNA, 21 bp, synthetic construct
  source 1..21: mol_type = other DNA
aagcctctccttcctcctgta

SEQ ID NO: 5 - DNA, 22 bp, synthetic construct
  source 1..22: mol_type = other DNA
aggattccgccttgttggtctg

SEQ ID NO: 6 - DNA, 22 bp, synthetic construct
  source 1..22: mol_type = other DNA
cggcaaagaatggacggtcctt

SEQ ID NO: 7 - RNA, 19 nt, synthetic construct
  source 1..19: mol_type = other RNA
aataagaggtggacacaac

SEQ ID NO: 8 - RNA, 19 nt, synthetic construct
  source 1..19: mol_type = other RNA
ctgaagaacacctgaagca

SEQ ID NO: 9 - DNA, 21 bp, synthetic construct
  source 1..21: mol_type = other DNA
gaaatcagcctatggtttaag

(19) United States
(12) Patent Application Publication
(10) Pub. No.: US 2026/0248768 A1
(43) Pub. Date: Aug. 27, 2026

(54) MYC PROGRAM AS A MARKER OF RESPONSE TO ENZALUTAMIDE IN PROSTATE CANCER

(71) Applicants: Rutgers, The State University of New Jersey, New Brunswick, NJ (US); Northwestern University, Evanston, IL (US)

(72) Inventors: Antonina Mitrofanova, New York, NY (US); Sukanya Panja, Harrison, NJ (US); Edward Schaeffer, Chicago, IL (US); Sarki Abdulkadir, Chicago, IL (US); Vishal Kothari, Chicago, IL (US)

(73) Assignees: Rutgers, The State University of New Jersey, New Brunswick, NJ (US); Northwestern University, Evanston, IL (US)

(21) Appl. No.: 18/854,762

(22) PCT Filed: Apr. 7, 2023
(86) PCT No.: PCT/US2023/065533

§ 371 Date: Oct. 7, 2024

Related U.S. Application Data
(60) Provisional application No. 63/329,074, filed on Apr. 8, 2022.

(51) Int. Cl. A61K 31/4166; A61K 31/58; C12Q 1/6886

(52) U.S. Cl. CPC A61K 31/4166; A61K 31/58; C12Q 1/6886; C12Q 2600/106; C12Q 2600/158

(57) ABSTRACT

Provided herein are methods of identifying a subject with prostate cancer (such as a human or veterinary subject) who will respond to enzalutamide therapy. In particular examples, the methods can determine with high accuracy whether a subject is likely to respond to enzalutamide therapy. Also provided are methods for treating a subject who is likely to respond to enzalutamide, for example by administering enzalutamide to the subject.

### CROSS REFERENCE TO RELATED APPLICATION

This application claims the benefit of U.S. Provisional Application No. 63/329,074, filed Apr. 8, 2022, which is incorporated herein by reference in its entirety.

### ACKNOWLEDGMENT OF GOVERNMENT SUPPORT

This invention was made with government support under grant number R01LM013236-01 awarded by the National Institutes of Health. The government has certain rights in the invention.

### FIELD

This disclosure relates to methods of treating and identifying subjects with prostate cancer that will respond to enzalutamide treatment.

### BACKGROUND

Since the seminal discovery of the central role of androgen receptor (AR) in prostate tumor growth and progression, androgen-deprivation therapy (ADT, an AR blockade therapy), has been the mainstay of treatment for prostate cancer (PCa). Although approximately 80% of patients initially respond to ADT, a large subset of patients relapse and progress to a more aggressive castration resistant prostate cancer (CRPC). Regardless of the continuous androgen deprivation, patients that develop CRPC retain a high level of AR signaling, which could be due to AR overexpression, AR mutations, or other mechanisms such as upregulation of glucocorticoid receptor that activates a subset of AR target genes. To overcome this problem, more potent AR inhibitors, such as ARSIs (androgen receptor signaling inhibitors), which includes Enzalutamide, Abiraterone, and Apalutamide, have been introduced for CRPC treatment.

While Enzalutamide (an AR signaling inhibitor that can block binding of androgen to androgen receptors with high affinity and can also inhibit AR nuclear translocation and AR binding to DNA) is one of the most commonly used ARSI that has shown to improve patient survival, nearly half of CRPC patients do not respond to it and develop resistance in approximately 8 months to 1.5 years. Unfortunately, CRPC patients that fail Enzalutamide treatment are left with no targeted therapeutic option, and progress to more lethal, metastatic form of CRPC and eventual death. Hence heterogeneity in response to Enzalutamide by CRPC patients pose a critical challenge in prostate cancer management.

### SUMMARY

There is a critical need for comprehensive analysis of clinical samples to elucidate mechanisms that govern response to Enzalutamide, with a special emphasis to identify readily targetable oncogenic drivers to improve the clinical outcomes. As disclosed herein, MYC-associated mechanisms serve as a biomarker of primary resistance to Enzalutamide and can identify patients that are at risk of developing resistance and that should potentially be offered alternative line of treatment. Moreover, the studies disclosed herein indicate that therapeutic targeting of MYC-associated mechanisms constitute a valuable primary treatment strategy for these patients and provides a potential secondary rescue therapy for patients that failed Enzalutamide.

Provided herein are methods of treating a subject with prostate cancer. In some implementations, the methods include measuring expression of one or more enzalutamide resistance-related molecules from one or more enzalutamide resistance-related molecular pathways and/or transcriptional regulatory programs in a sample (such as a prostate cancer sample) obtained from the subject, wherein the enzalutamide resistance-related molecular pathways or transcriptional regulatory programs include a Myc molecular pathway, a NME2 transcriptional regulatory program, or any combination thereof; and administering enzalutamide to the subject with prostate cancer, wherein expression of the one or more enzalutamide resistance-related molecules is similar to a control representing expression for the one or more enzalutamide resistance-related molecules expected in a sample from a subject who positively responds to enzalutamide therapy; or administering an androgen receptor signaling inhibitor that is not enzalutamide (such as abiraterone) to the subject with prostate cancer, wherein expression of the one or more enzalutamide resistance-related molecules differs from a control representing expression for the one or more enzalutamide resistance-related molecules expected in a sample from a subject who positively responds to enzalutamide therapy, thereby treating the subject with prostate cancer. In some examples, treating the subject with enzalutamide therapy occurs wherein the subject is identified as a subject who responds positively to enzalutamide therapy with a p value of 0.01 or less. In one example, p value is 0.0035

Also provided are methods of identifying a subject with prostate cancer who will respond positively to enzalutamide therapy. In some implementations, the methods include measuring expression of one or more enzalutamide resistance-related molecules from one or more enzalutamide resistance-related molecular pathways or transcriptional regulatory programs in a sample (such as a prostate cancer sample) obtained from the subject, wherein the enzalutamide resistance-related molecular pathways or transcriptional regulatory programs include a Myc molecular pathway, a NME2 transcriptional regulatory program, or any combination thereof, wherein expression of the enzalutamide resistance-related molecules is similar to a control representing expression for the enzalutamide resistance-related molecules expected in a sample from a subject who positively responds to enzalutamide therapy, thereby identifying a subject with prostate cancer who will respond positively to enzalutamide therapy. In some examples, the subject is identified as a subject who will respond positively to enzalutamide therapy with a p value of 0.01 or less. In one example, the p value is 0.0035

In some implementations of the disclosed methods, the one or more enzalutamide resistance-related molecules from one or more enzalutamide resistance-related molecular pathways or transcriptional regulatory programs include one or more of MYC, SLC19A1, MRTO4, TMEM97, RRP9, PES1, TFB2M, EXOSC5, IPO4, NDUFAF4, NOC4L, SRM, PA2G4, GNL3, NOLC1, WDR43, RABEPK, NOP16, TBRG4, DDX18, NIP7, WDR74, BYSL, HSPD1, PLK4, NOP2, PPAN, NOP56, RCL1, NPM1, AIMP2, RRP12, PPRC1, TCOF1, MCM5, HK2, CBX3, PLK1, PHB, MCM4, CDK4, DUSP2, MYBBP1A, UTP20, PRMT3, FARSA, MAP3K6, LAS1L, PUS1, HSPE1, SLC29A2, DCTPP1, SUPV3L1, SORD, IMP4, GRWD1, UNG, and MPHOSPH10 from the Myc molecular pathway, NME2, or any combination thereof. In some implementations, the expression is mRNA expression. In some examples, expression of the one or more enzalutamide resistance-related molecules from one or more enzalutamide resistance-related molecular pathways or transcriptional regulatory programs is measured by RNAseq or qRT-PCR.

In some implementations, the subject who positively responds to enzalutamide therapy is a subject who does not develop resistance to enzalutamide therapy. In other implementations, a subject that responds positively to enzalutamide therapy is a subject with a prostate cancer that is reduced in size by at least 20%, at least 50%, at least 80%, at least 90%, at least 95%, at least 98%, or even at least 100%, following administration of the enzalutamide therapy, as compared to no treatment with the enzalutamide therapy; with a metastasis that is reduced in size by at least 20%, at least 50%, at least 80%, at least 90%, at least 95%, at least 98%, or even at least 100%, following administration of the enzalutamide therapy, as compared to no treatment with the enzalutamide therapy; has an increase in survival time following administration of the enzalutamide therapy, as compared to no treatment with the enzalutamide therapy; has a reduction of at least 65%, at least 85%, at least 90%, at least 95%, or at least 98%, in developing resistance to the enzalutamide therapy; or combinations thereof, within one year of starting treatment with the enzalutamide therapy.

In some implementations, the methods of treating a subject with prostate cancer further include administering subject an inhibitor of a Myc molecular pathway or a NME2 transcriptional regulatory program to the subject.

Also provided are methods of treating a subject with prostate cancer (such as a subject that has received enzalutamide therapy and undergone disease progression and having a prostate cancer expressing increased Myc and NME2 compared to a sample from a subject who positively responds to enzalutamide therapy), which include administering to the subject an inhibitor of a Myc molecular pathway or a NME2 A transcriptional regulatory program, thereby treating the enzalutamide-resistant prostate cancer. In some implementations, the inhibitor of the Myc molecular pathway inhibits Myc expression or activity, for example Myc-i975. In other implementations, the inhibitor of the NME2 transcriptional regulatory program inhibits NME2 expression or activity (such as an antisense oligonucleotide or siRNA that specifically binds to an NME 2 nucleic acid). In some implementations, the methods further comprise treating the subject with enzalutamide.

The foregoing and other features of the disclosure will become more apparent from the following detailed description, which proceeds with reference to the accompanying figures.

### BRIEF DESCRIPTION OF THE DRA WINGS

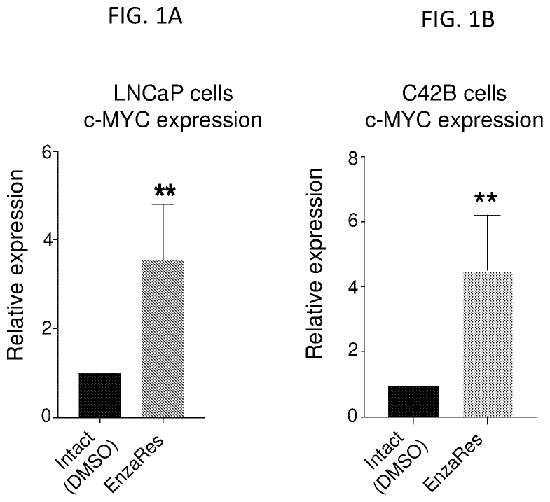

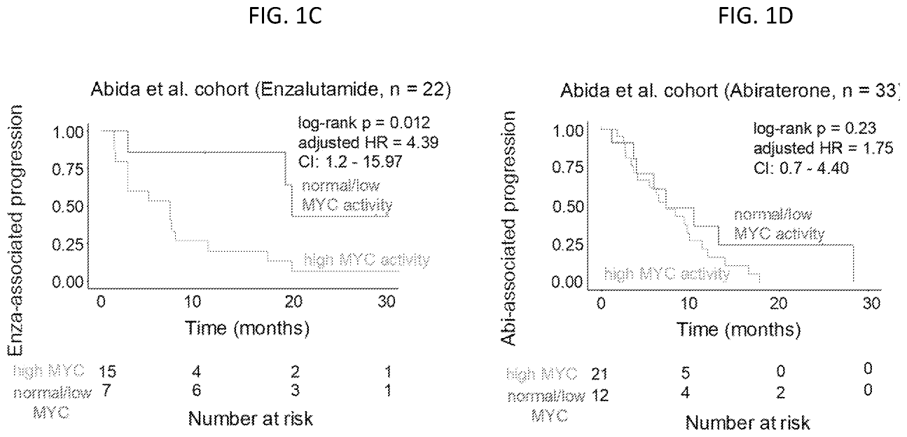

FIGS. 1A-1D show MYC pathway activity is specific for predicting response to Enzalutamide in CRPC patients. (FIGS. 1A-1B) c-MYC expression in Intact (DMSO treated) and Enzalutamide-resistant (EnzaRes) (FIG. 1A) LNCaP and (FIG. 1B) C42B cells as shown using the qRT-PCR. P-values were estimated using a one-tailed Welch t-test. ** p-value≤0.01. (FIGS. 1C-1D) Kaplan-Meier survival analysis comparing CRPC patients that received (FIG. 1C) Enzalutamide or (FIG. 1D) Abiraterone after sample collection from the Abida et al. cohort with high and normal/low MYC pathway activity levels. Log-rank p-value, adjusted HR (hazard ratio), and CI (confidence interval) are indicated.

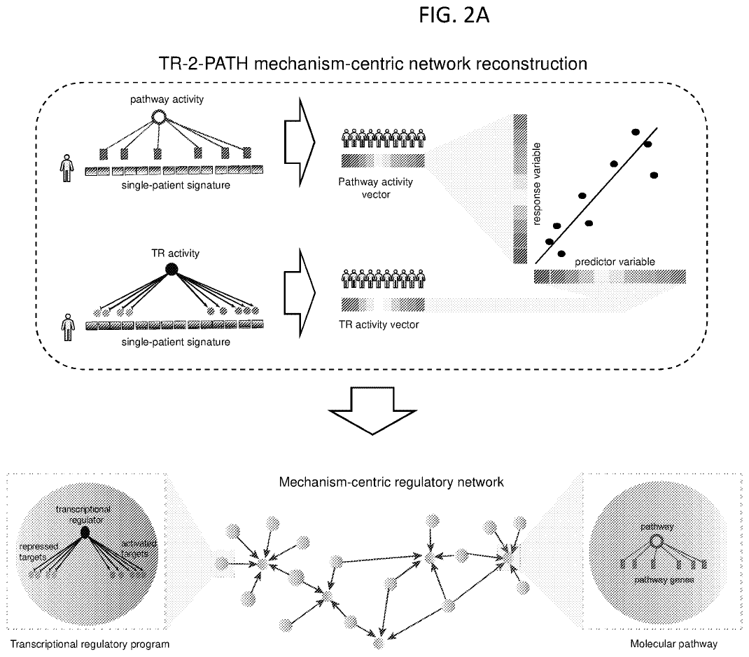

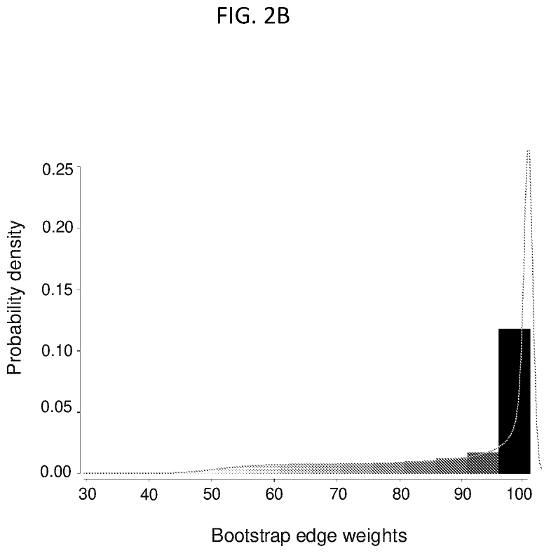

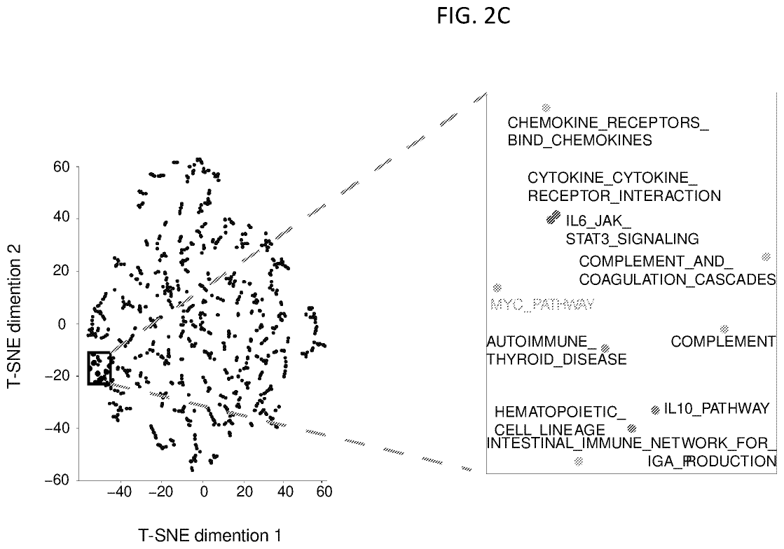

FIGS. 2A-2D show reconstruction of a mechanism-centric systems regulatory network for CRPC patients. (FIG. 2A) Schematic representation of the TR-2-PATH workflow. (First row) Single-patient pathway enrichment analysis and single-patient transcriptional regulatory analysis identifies pathway activity vector and transcriptional regulatory activity vector respectively, pairs of which are then subjected to linear regression analysis to reconstruct a mechanism-centric regulatory network. (Second row) In the network, transcriptional regulatory programs are represented as orange nodes and molecular pathways as green nodes. An edge (black arrow) illustrates that a significant relationship was defined between a transcriptional regulatory program and molecular pathway. (FIG. 2B) Distribution of edge weights across the network, as defined by the bootstrap analysis. The x-axis corresponds to the edge weight and the y-axis to its frequency (probability). (FIG. 2C) (Left) t-SNE clustering of molecular pathways (dots), based on the weights of their incoming edges. (Right) Pathways around MYC are shown as a zoom-in and MYC pathway is shown. FIG. 2D: Bootstrap consistency is confirmed by the similarity of significant edge distributions across bootstrap runs. Consistency of bootstrap runs in SU2C East Coast cohort is demonstrated through comparison of the distribution of number of significant edges for each pathway across runs. Leftmost indicates results from the original (whole) SU2C East Coast dataset and remaining indicates results from the bootstrap runs on the same dataset.

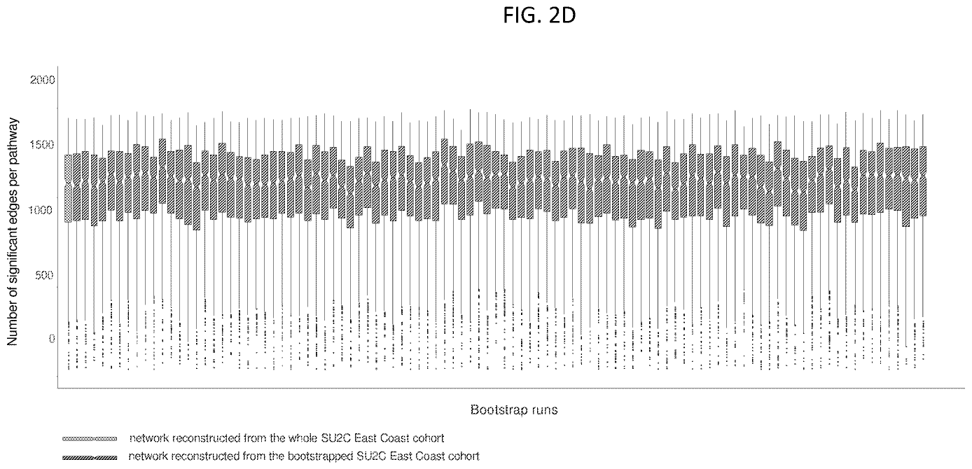

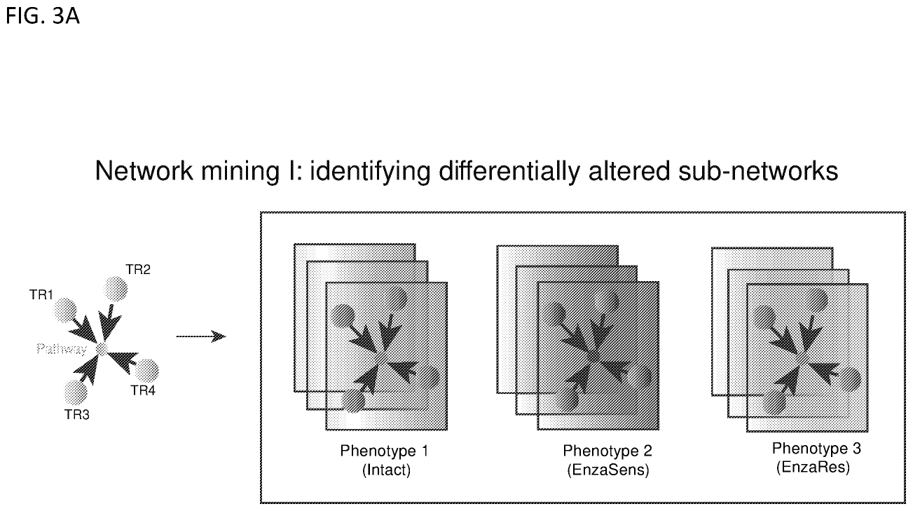

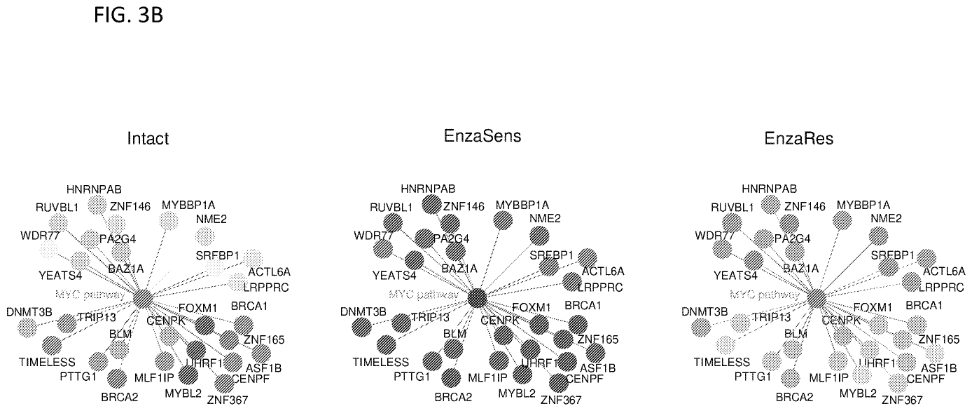

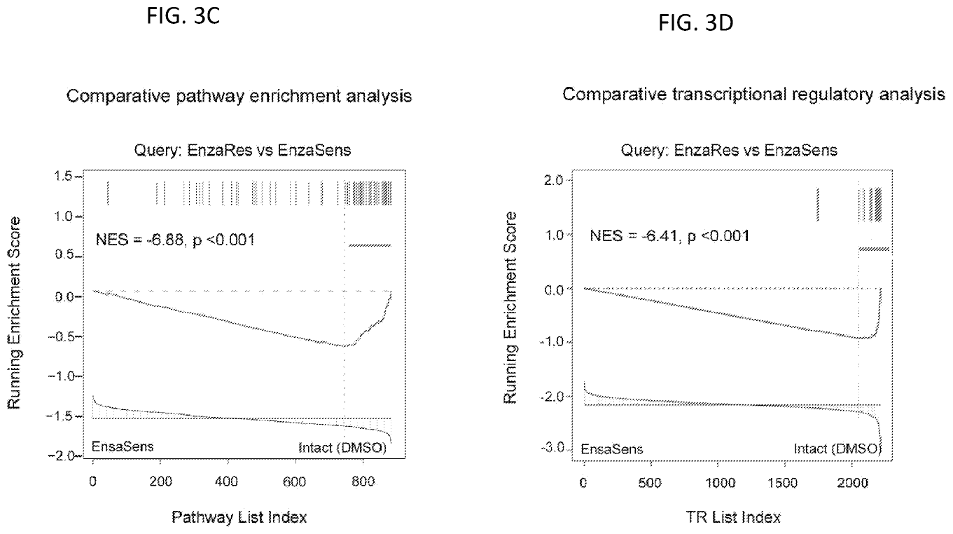

FIGS. 3A-3D show network mining I: Identification of upstream transcriptional regulatory programs that affect MYC pathway and are associated with response to Enzalutamide treatment. (FIG. 3A) Schematic representation of the changes in activity levels of molecular pathways and their upstream transcriptional regulatory programs (sub-networks) as they transition from Intact (treated with DMSO) to Enzalutamide-sensitive (EnzaSens) to Enzalutamide-resistant (EnzaRes) phenotypes. TR and pathway activities are up-regulated in phenotypes 1 and 3 and down-regulated in phenotype 2. (FIG. 3B) Identified upstream transcriptional regulatory programs (MYC-centered sub-network) associated with Enzalutamide treatment affecting MYC pathway, depicted across Intact, EnzaSens, and EnzaRes phenotypes. TR and pathway activities are up-regulated in Intact and EnzaRes and down-regulated in EnzaSens. FIGS. 3C-3D show mechanism-centric network mining identifies molecular pathways and TR programs that govern progression to Enzalutamide resistance. (FIG. 3C) GSEA performed on pathway activity levels between a reference signature comparing Enzalutamide-sensitive (EnzaSens, n=4) to Intact (treated with DMSO, n=4) samples and query signature comparing Enzalutamide-resistant (EnzaRes, n=4) and Enzalutamide-sensitive (EnzaSens, n=4) samples (query set was defined as up-regulated pathways at p-value <0.05). GSEA NES (normalized enrichment score) and p-value were estimated using 1,000 pathway permutations in the reference signature. (FIG. 3D) GSEA performed on TR activity levels between a reference signature comparing Enzalutamide-sensitive (EnzaSens, n=4) to Intact (treated with DMSO, n=4) samples and query signature comparing Enzalutamide-resistant (EnzaRes, n=4) and Enzalutamide-sensitive (EnzaSens, n=4) samples (query set was defined as up-regulated TRs at p-value <0.05). NES (normalized enrichment score) and p-value were estimated using 1,000 TR permutations in the reference signature.

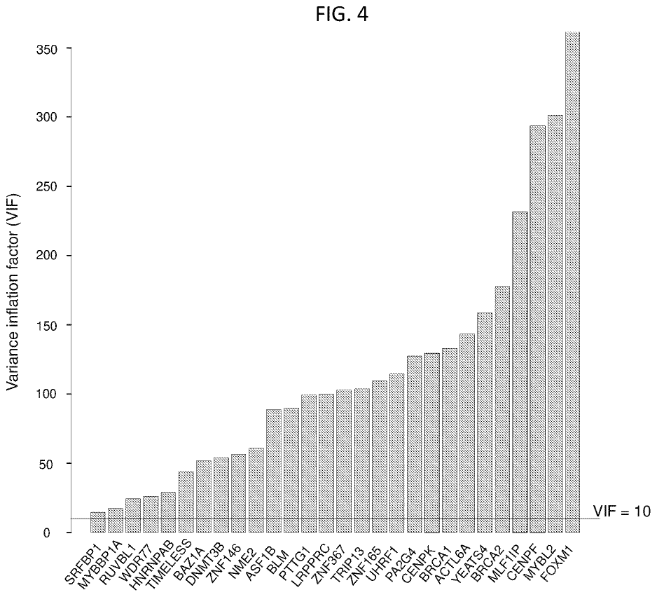

FIG. 4 shows VIF analysis identifies multi-collinearity between the transcriptional regulatory programs affecting MYC pathway. Bar plot representation of VIF (variance inflation factor) analysis. Each bar corresponds to VIF value for the indicated transcriptional regulatory program (shown on x-axis).

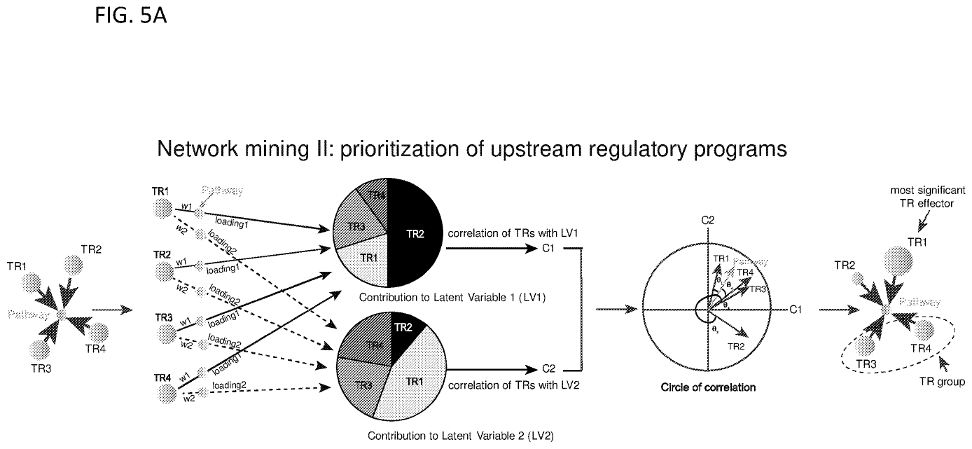

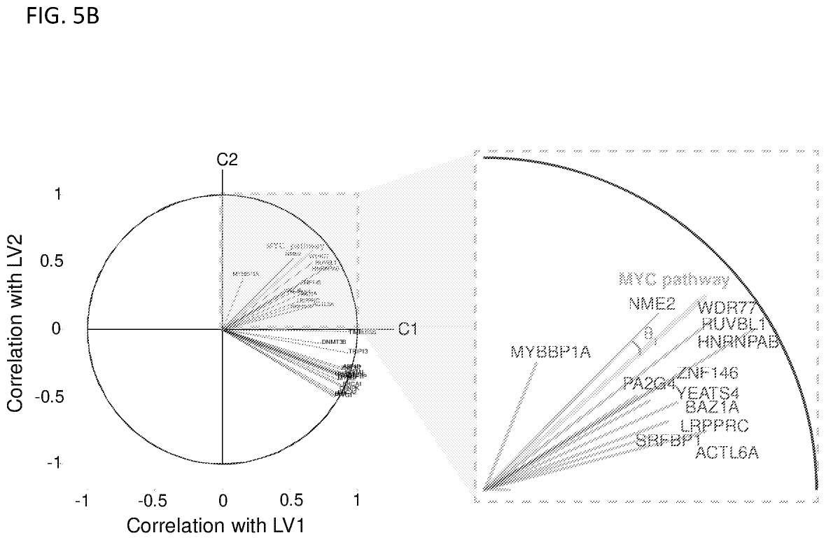

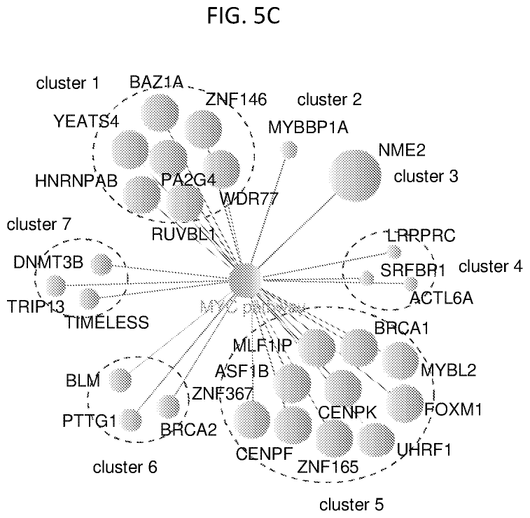

FIGS. 5A-5C shows Network mining II: NME2 has the largest independent effect on MYC pathway. (FIG. 5A) Schematic representation of the PLS-inspired approach to prioritize TR programs, based on their effect on a molecular pathway of interest. (Left) TR activity vectors are utilized as inputs, which are then regressed on a pathway to identify non-collinear latent variables (pie charts), which include a linear combination of TR programs, based on their effect on the pathway (slices in each pie). (Middle) These latent variables are utilized to build a circle of correlation, which depicts the relationship between each latent variable and each TR and pathway. (Right) Effect scores are defined to group and prioritize TRs, based on their effect on a pathway. (FIG. 5B) A circle of correlation is utilized to determine the degree of closeness between TR programs and the MYC pathway, based on their effect on each latent variable. (FIG. 5C) Grouping and prioritization of the MYC upstream TR programs. Circle sizes correspond to the TR effect scores. NME2 is determined to have the most significant effect on MYC pathway.

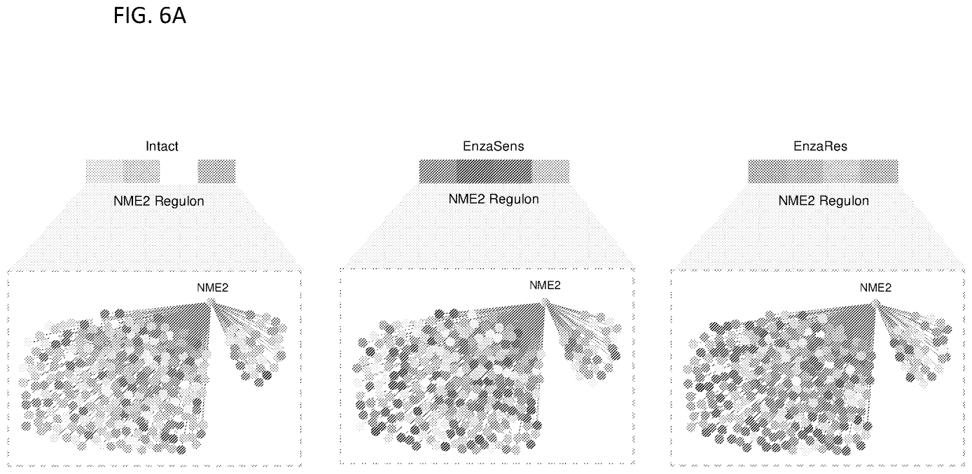

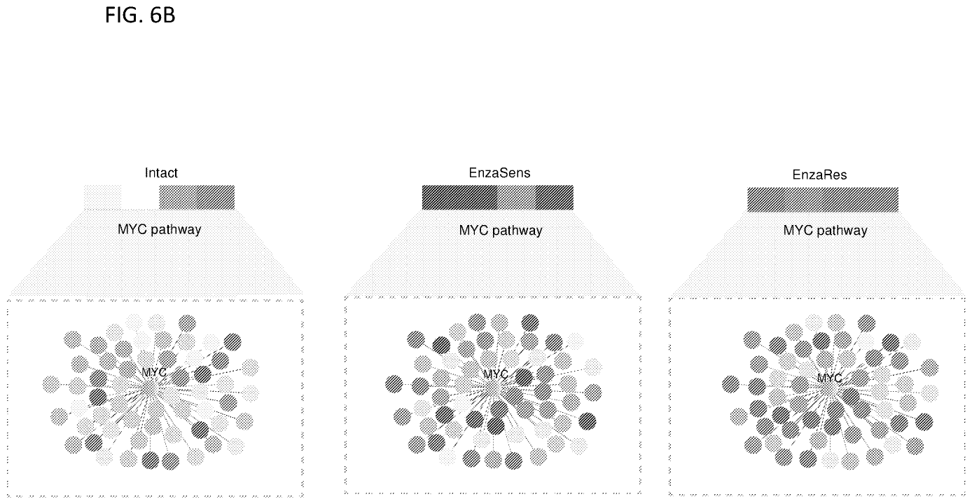

FIGS. 6A-6B show activity levels of NME2 transcriptional program and MYC molecular pathway reveal significant changes across Enzalutamide-related conditions. (FIG. 6A) Expression levels of NME2 activated targets (n=368) across Intact (treated with DMSO), Enza-sensitive (EnzaSens), and Enza-resistant (EnzaRes) phenotypes. (FIG. 6B) Expression levels of MYC pathway genes (n=57) across Intact (treated with DMSO), Enza-sensitive (EnzaSens), and Enza-resistant (EnzaRes) phenotypes.

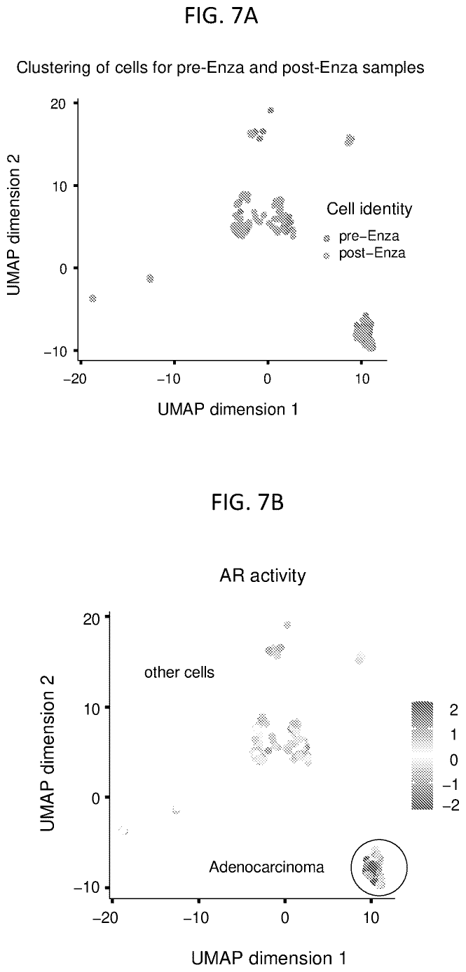

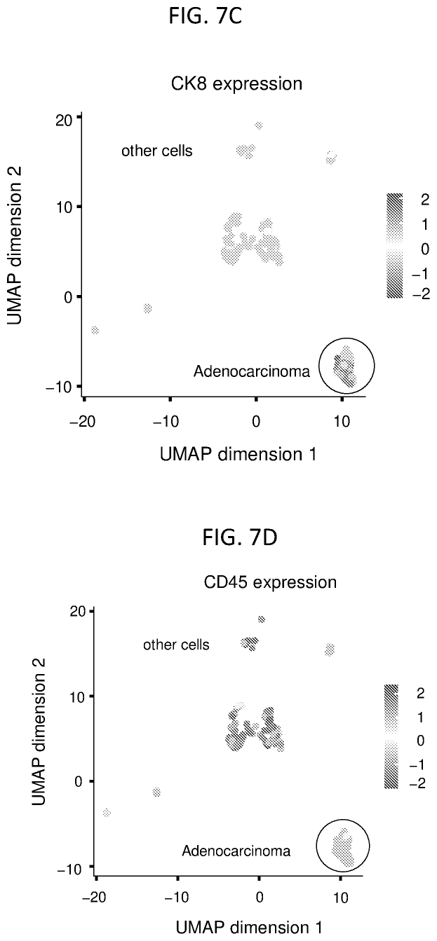

FIGS. 7A-7D show upregulation of AR, CK8, and CD45 identify adenocarcinoma prostate cancer cells in pre- and post-Enzalutamide conditions. UMAP representation of (FIG. 7A) pre- and post-Enza cell populations, (FIG. 7B) AR activity levels, (FIG. 7C) CK8 expression levels, and (FIG. 7D) CD45 expression levels, identifying a group of adenocarcinoma prostate cells (circle).

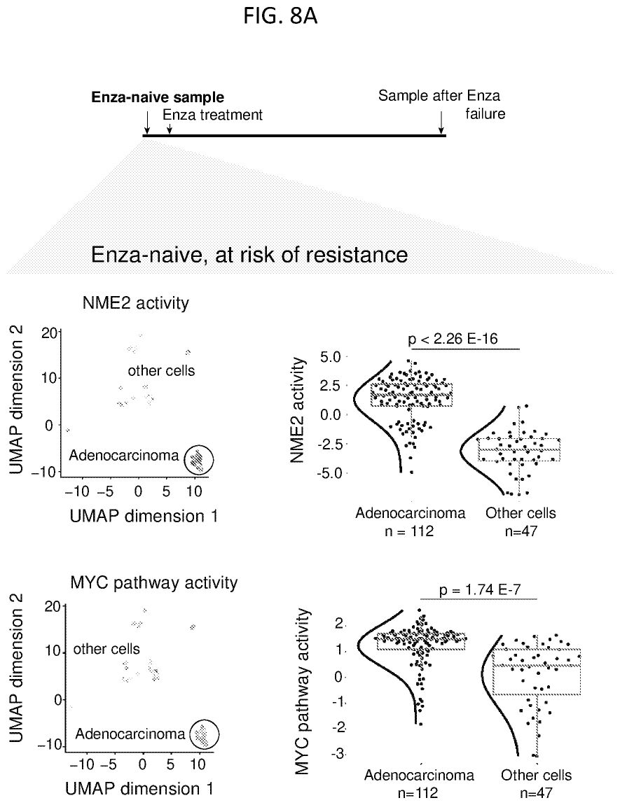

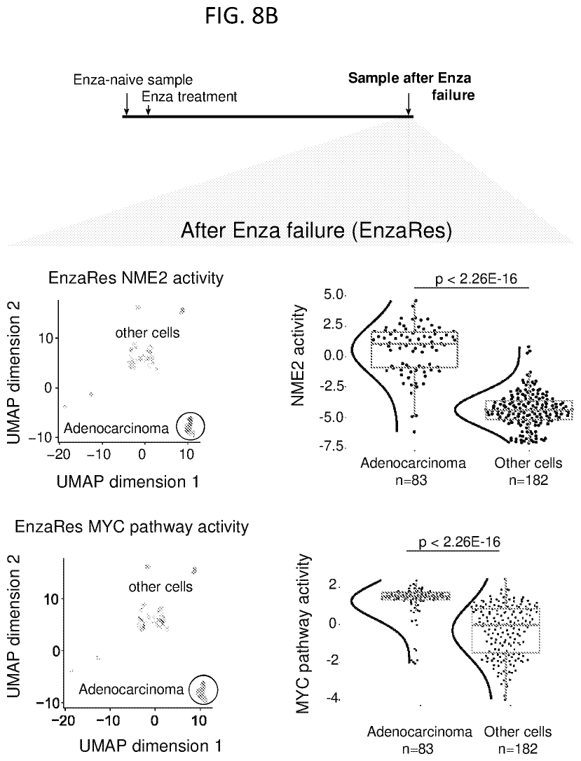

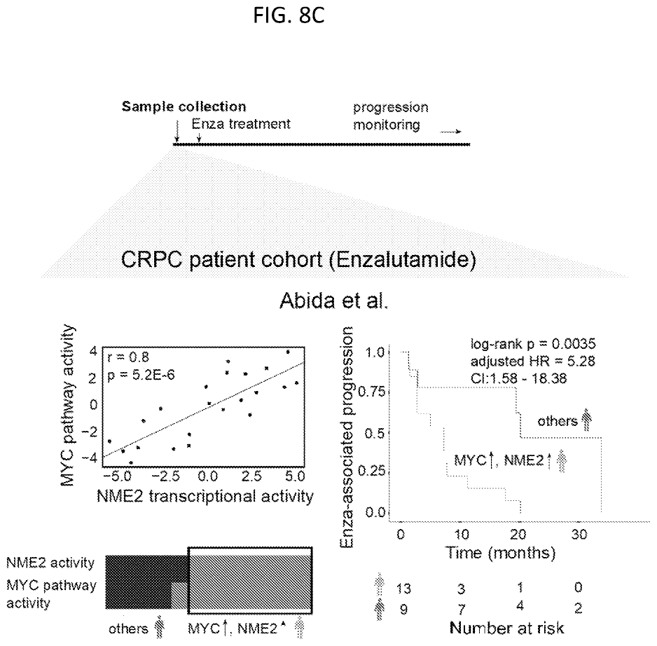

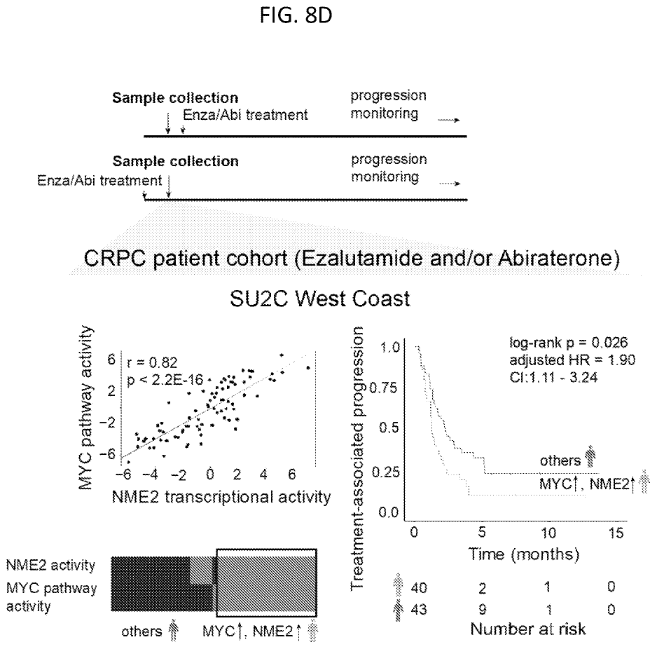

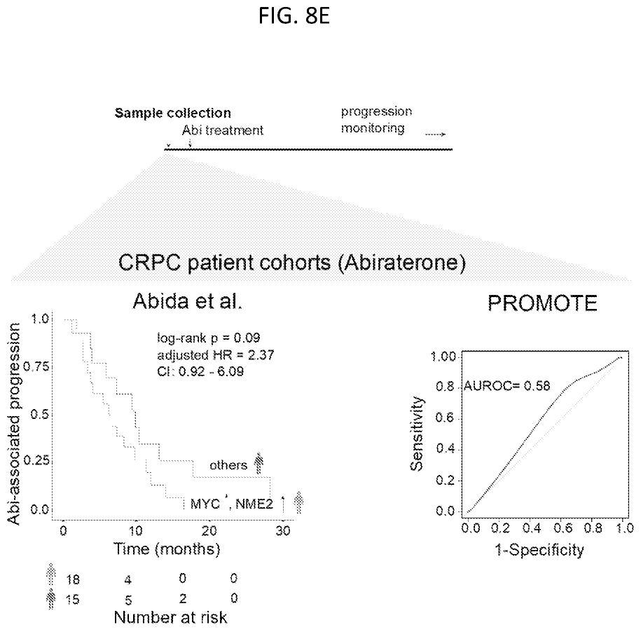

FIGS. 8A-8E show activity of MYC and NME2 predict poor response to Enzalutamide. (FIGS. 8A-8B) Comparing NME2 TR and MYC pathway activity between adenocarcinoma cells and other cells in neoadjuvant and adjuvant samples of a CRPC patient obtained from He et al. P-value was estimated using a one-tailed Welch t-test. (FIG. 8C) (Left, top) Pearson correlation analysis between NME2 TR and MYC pathway activity in the Abida et al. cohort subjected to adjuvant Enzalutamide. Pearson r and p-value are indicated. (Left, bottom) Patients with high-MYC and high-NME2 and the rest of the patients (others) were identified. (Right) Kaplan-Meier survival analysis, comparing high-MYC and high-NME2 group to the rest of the patients in Abida et al. cohort subjected to adjuvant Enzalutamide. Log-rank p-value, adjusted HR (hazard ratio), and CI (confidence interval) are indicated. (FIG. 8D) (Left, top) Pearson correlation analysis between NME2 TR and MYC pathway activity in SU2C West Coast cohort subjected to Enzalutamide and/or Abiraterone either before or after sample collection. Pearson r and p-value are indicated. (Left, bottom) Patients with high-MYC and high-NME2 and the rest of the patients were identified. (Right) Kaplan-Meier survival analysis, comparing high-MYC and high-NME2 group to the rest of the patients in SU2C West Coast cohort. Log-rank p-value, adjusted HR (hazard ratio), and CI (confidence interval) are indicated. (FIG. 8E) (Left) Kaplan-Meier survival analysis, comparing high-MYC and high-NME2 group to the rest of the patients in Abida et al. cohort subjected to adjuvant Abiraterone. Log-rank p-value, adjusted HR, and CI are indicated. (Right) ROC analysis using NME2 TR and MYC pathway activity levels to compare CRPC patients with poor and favorable Abiraterone response from PROMOTE cohort. AUROC (area under ROC) is indicated.

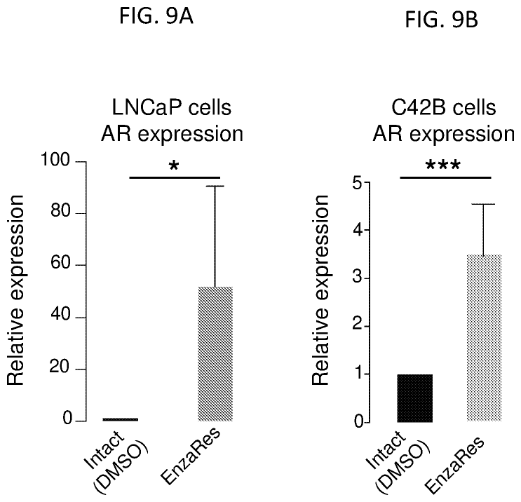

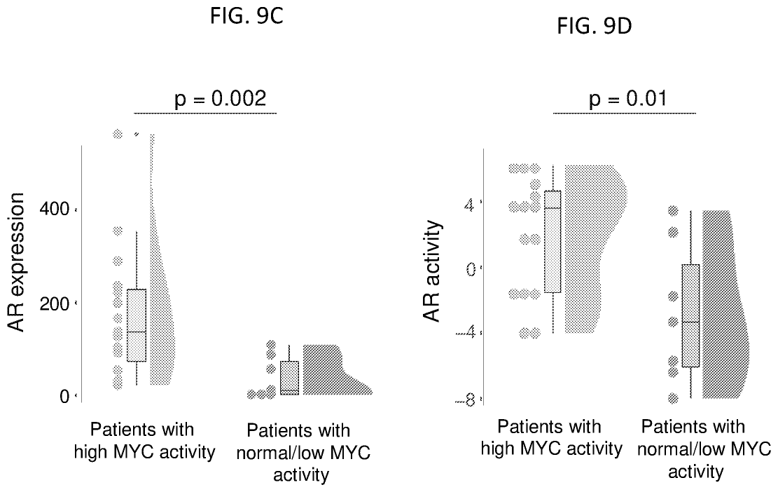

FIGS. 9A-9D show AR expression and activity are higher in Enzalutamide resistant phenotypes. (FIGS. 9A-9B) AR expression in Intact (treated with DMSO) and Enzalutamide resistant (EnzaRes) (FIG. 9A) LNCaP and (FIG. 9B) C42B cell lines, as shown using qRT-PCR. (FIG. 9C) Comparing AR expression and (FIG. 9D) AR activity in CRPC patients from Abida et al patient cohort with high MYC activities and normal/low MY activities. P-values were estimated using a one-tailed Welch t-test. * p<0.05, *** p<0.001

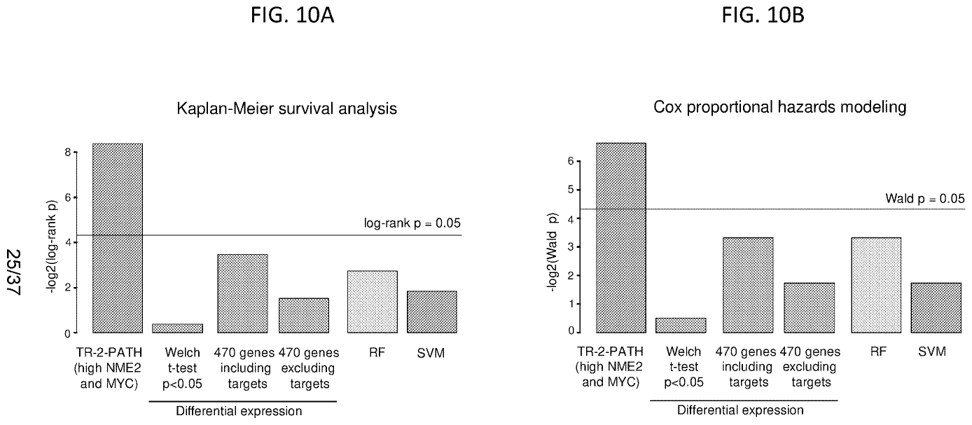

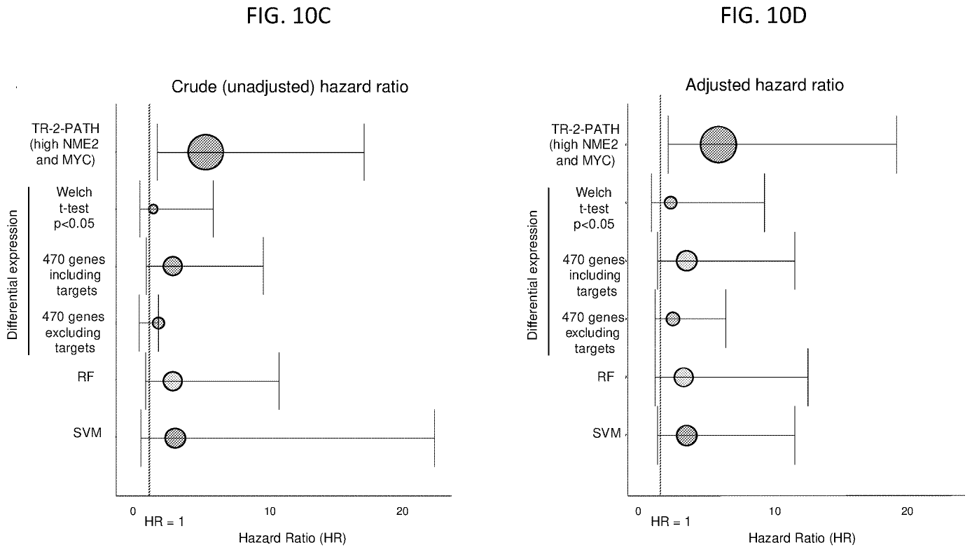

FIGS. 10A-10D show comparative analysis to different computational methods demonstrates superiority of TR-2-PATH approach. Comparison of different methods with respect to their ability to predict Enzalutamide resistance. Methods include TR-2-PATH (high-MYC and high-NME2), differential expression analysis between Intact, EnzaSens and EnzaRes phenotypes (Welch t-test p-value <0.05, top 470 genes including NME2 TR targets and MYC pathway genes, top 470 genes excluding NME2 TR targets and MYC pathway genes), top 10 predictions from Random (survival) Forests (RF) method, and top 10 predictions from Support Vector Machine (SVM) method. Comparison among methods was done using: (FIG. 10A) Kaplan-Meier survival analysis (log-rank p-value indicated), (FIG. 10B) Cox modeling (Wald p-value indicated), (FIG. 10C) crude (unadjusted) hazards ratio, and (FIG. 10D) adjusted (for age at diagnosis and Gleason score) hazards ratio. Circles correspond to hazard ratio values and whiskers to their Confidence Intervals, HR=1 is indicated as a vertical line in (FIGS. 10C-10D).

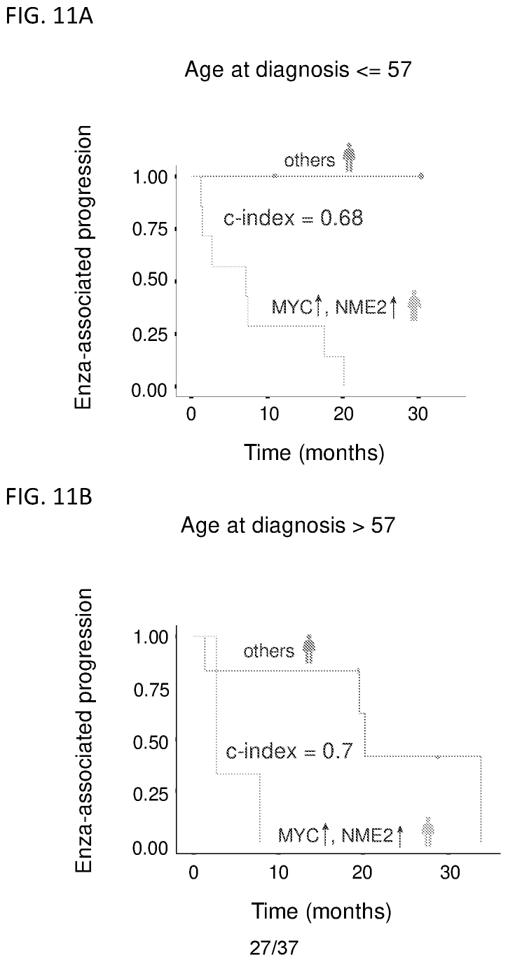

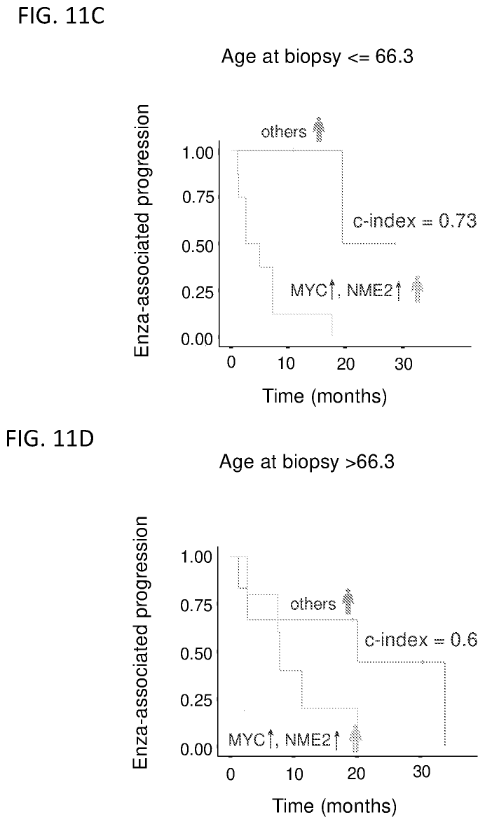

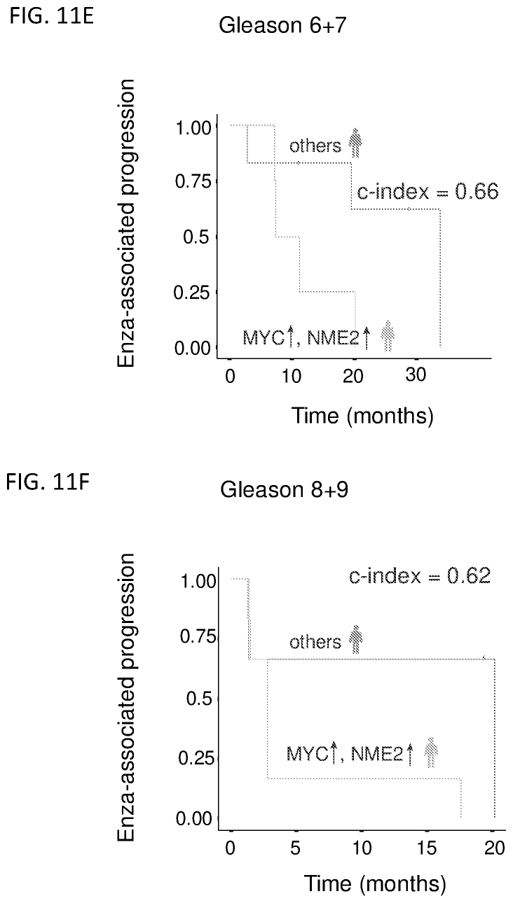

FIGS. 11A-11F show Kaplan-Meier survival analysis stratified by age at diagnosis, age at biopsy, and Gleason score demonstrates independent predictive ability of NME2 and MYC. Kaplan-Meier survival analysis for CRPC patients, that were subjected to Enzalutamide after sample collection, in Abida et al. cohort, stratified based on (FIGS. 11A-11B) median age at diagnosis: ≤57 (FIG. 11A) and >57 (FIG. 11B). (FIGS. 11C-11D) median age at biopsy: ≤66.3 (FIG. 11C) and >66.3 (FIG. 11D) and (FIGS. 11E-11F) Gleason score: Gleason 6 and 7 (FIG. 11E) and Gleason 8 and 9 (FIG. 11F). C-index (corresponding to AUROC) is indicated. Group 1 corresponds to patients with high NME2 transcriptional and high MYC pathway activity levels. The rest of the patients is represented by Group 2.

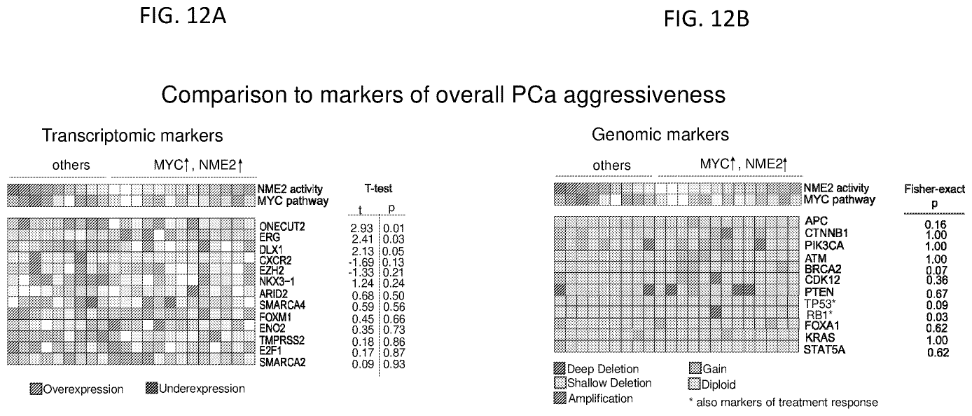

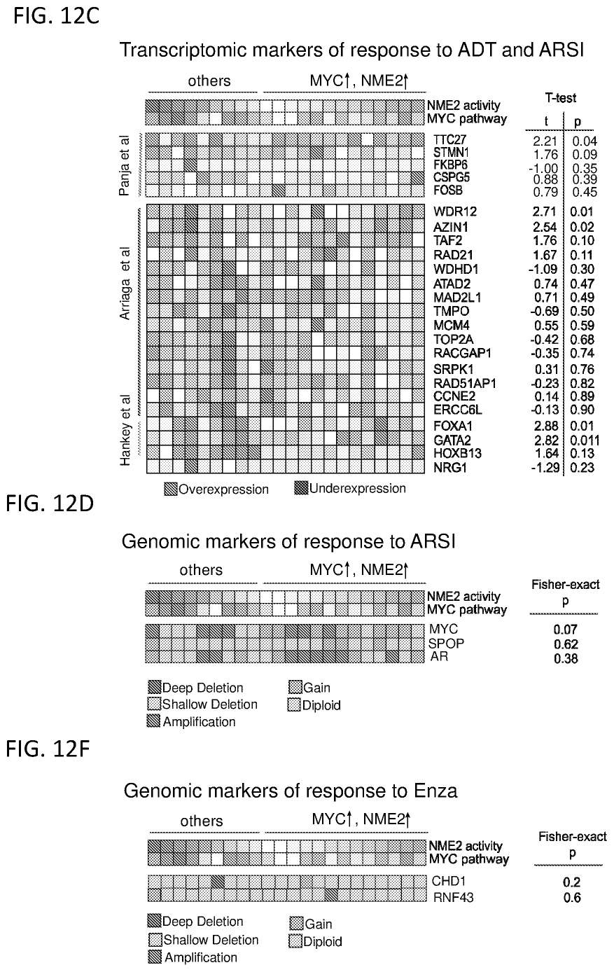

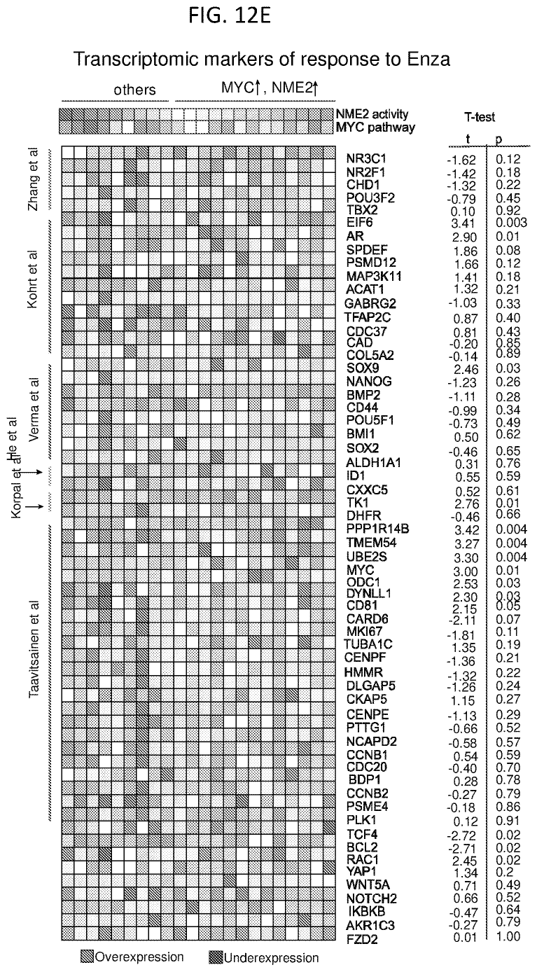

FIGS. 12A-12F show ability of MYC and NME2 to predict Enzalutamide-response outperforms known markers of PCa progression and treatment response. (FIGS. 12A-12B) Comparison of MYC and NME2 ability to predict response to Enzalutamide in Abida et al. cohort to known markers of PCa aggressiveness, including (FIG. 12A) transcriptomic and (FIG. 12B) genomic markers. (FIGS. 12C-12D) Comparison of MYC and NME2 ability to predict response to Enzalutamide in Abida et al. cohort to known markers of response to ADT and ARSIs including (FIG. 12C) transcriptomic and (FIG. 12D) genomic markers. (FIGS. 12E-12F) Comparison of MYC and NME2 ability to predict response to Enzalutamide in Abida et al. cohort to known markers of Enzalutamide-response, including (FIG. 12E) transcriptomic and (FIG. 12F) genomic markers. Two-tailed Welch t-test was utilized to calculate p-values to estimate the difference in expression levels between high-MYC and high-NME2 group and the rest of the patients for transcriptomic markers in FIGS. 12A, C, and E. Fisher-exact test was utilized to calculate p-values to estimate the difference in the frequency/occurrence of any genomic alterations between high-MYC and high-NME2 group and the rest of the patients for genomic markers in FIGS. 12B, D, and F.

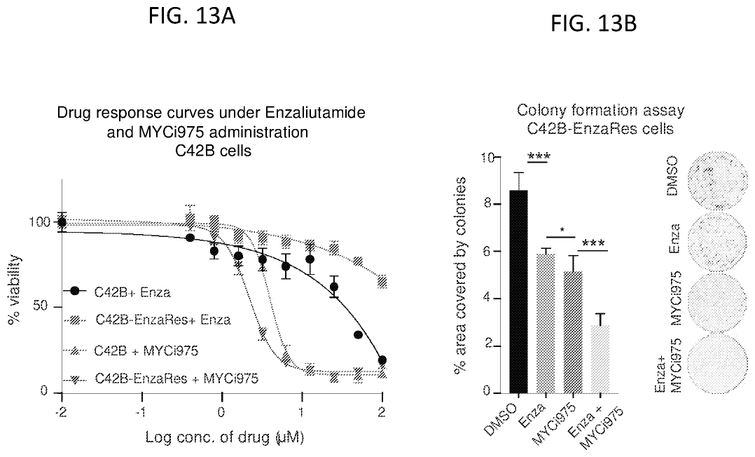

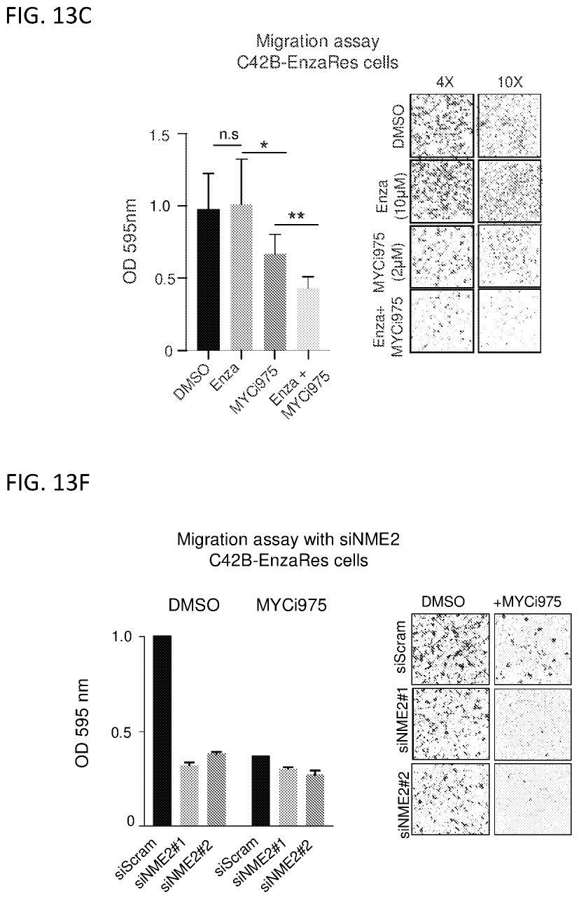

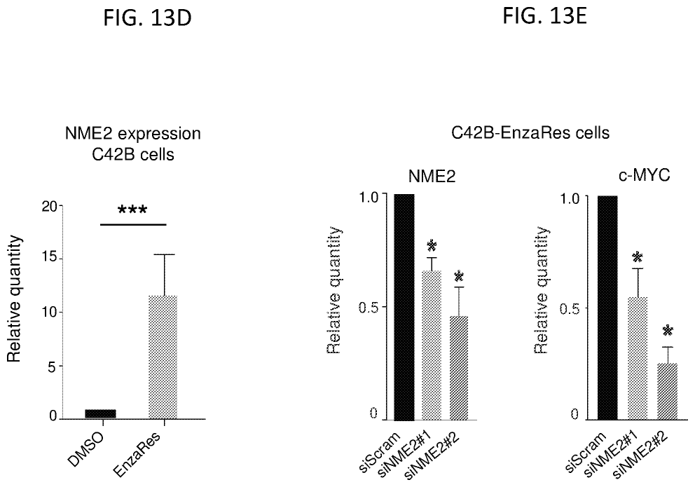

FIGS. 13A-13F show MYC targeting is beneficial for patients in Enzalutamide-resistant conditions. (FIG. 13A) Drug sensitivity curves of Enzalutamide-naïve, or Enzalutamide-resistant (EnzaRes) C42B cells treated with MYCi975. (FIG. 13B) Colony formation assay using Enzalutamide-resistant (EnzaRes) C42B cells in Intact (treated with DMSO), treated with Enzalutamide (10 μM), MYCi975 (2 μM), or a combination of Enzalutamide+MYCi975 (10 μM+2 μM). Cells were grown in the presence of respective drugs. Representative images are shown. P-value was estimated using a one-tailed Welch t-test. (FIG. 13C) Boyden chamber-based in vitro migration assay using Enzalutamide-resistant (EnzaRes) C42B cells in Intact (treated with DMSO), treated with Enzalutamide (10 μM), MYCi975 (2 μM), or a combination of Enzalutamide+MYCi975 (10 μM+2 μM). Bars represent the quantification of Crystal Violet trapped by migrated cells. P-value was estimated using a one-tailed Welch t-test. (FIG. 13D) Expression of NME2 in Intact and Enzalutamide-resistant (EnzaRes) C42B cells, using the qRT-PCR. P-value was estimated using the one-tailed Welch t-test. (FIG. 13E) Two different siRNAs targeting NME2 were used to downregulate NME2 (left panel) and its effect on MYC expression using qRT-PCR is shown (right panel). (FIG. 13F) Boyden chamber-based in vitro migration assay using Enzalutamide-resistance (EnzaRes) C42B cells in Intact (treated with DMSO) or treated with a combination of Enzalutamide+MYCi975 (10 μM+2 μM) with or without knockdown of NME2. Bars represent the quantification of Crystal Violet trapped by migrated cells. * p-value <0.05, ** p-value≤0.01, *** p-value≤0.001

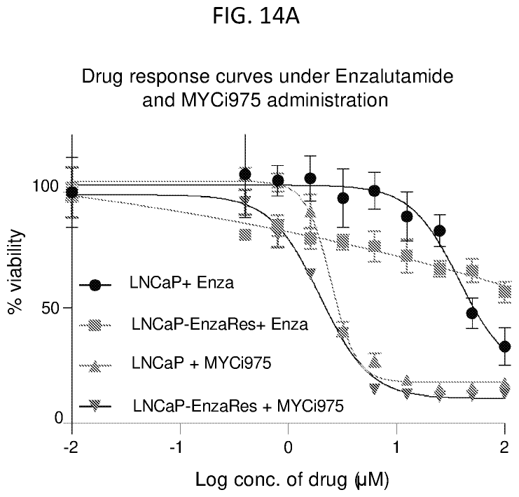

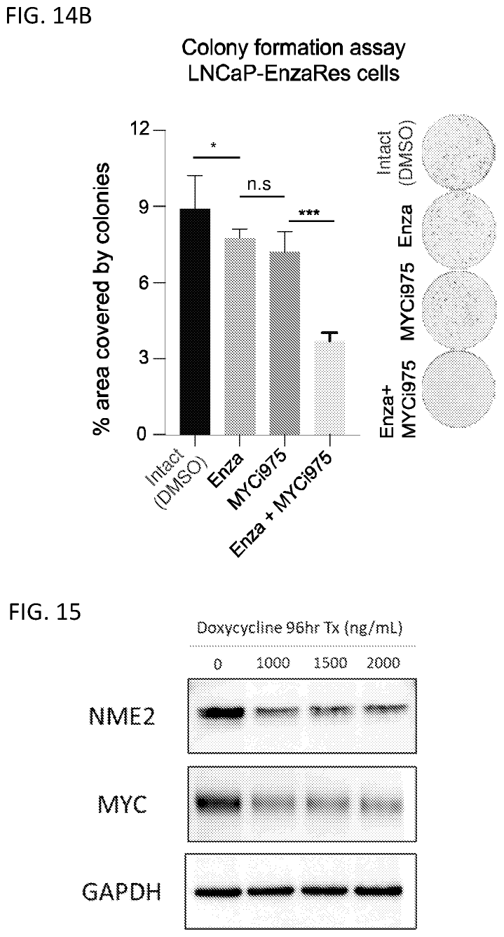

FIGS. 14A and 14B show MYC inhibition reduces viability and colony formation in Enzalutamide resistant conditions. (FIG. 14A) Drug response curves of Enzalutamide naïve LNCaP or Enzalutamide-resistant LNCaP cells treated with Enzalutamide and/or MYC-i975. (FIG. 14B) Colony formation assay using Enzalutamide-resistant LNCaP cells t (LNCaP-Enza-Res) in intact (treated with DMSO), treated with enzalutamide (10 μM), MYC-i975 (2 μM), or a combination of Enzalutamide+MYC-i975 (10 μM+2 μM). Cells were grown in the presence of respective drugs. Bars represent quantification of Crystal Violet trapped by migrated cells. P-value is estimated utilizing one-tailed Welch t-test. * p<0.05, *** p≤0.001.

FIG. 15 is a Western blot showing that inducible knockdown of NME2 in C4-2B cells suppresses MYC expression.

### SEQUENCES

Any nucleic acid and amino acid sequences listed herein are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.
SEQ ID NOs: 1 and 2 are forward and reverse primers for cMYC, respectively.
SEQ ID NOs: 3 and 4 are forward and reverse primers for androgen receptor (AR), respectively.
SEQ ID NOs: 5 and 6 are forward and reverse primers for NME2, respectively.
SEQ ID NOs: 7 and 8 are siRNAs targeting NME2.
SEQ ID NO: 9 is a shRNA targeting NME2.

### DETAILED DESCRIPTION

### I. Terms

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a cell” includes single or plural cells and is considered equivalent to the phrase “comprising at least one cell.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Dates of GenBank® Accession Nos. referred to herein are the sequences available at least as early as Apr. 8, 2022

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

In order to facilitate review of the various implementations of the disclosure, the following explanations of specific terms are provided.

Control: A reference standard. In some implementations, the control is a healthy subject. In other implementations, the control is a subject with a cancer, such as a prostate cancer. In some implementations, the control is a subject who responds positively to a therapy (for example, enzalutamide therapy), such as a subject who does not develop resistance to the therapy. In other implementations, the control is a subject who does not respond positively to a therapy (for example, enzalutamide therapy), such as a subject who develops resistance to the therapy. In still other implementations, the control is a historical control or standard reference value or range of values (e.g., a previously tested control subject with a known prognosis or outcome or group of subjects that represent baseline or normal values). A difference between a test subject and a control can be an increase or a decrease. The difference can be a qualitative difference or a quantitative difference, for example a statistically significant difference. Values that are “similar” between a test subject and a control can be values that are not statistically significantly different.

Detect: To determine if an agent (such as a signal; particular nucleotide; amino acid; nucleic acid molecule; and/or peptide or protein) is present or absent. In some examples, detection can include further quantification. For example, use of the disclosed methods in particular examples permits detection of nucleic acid expression in a sample.

Differential Expression: A nucleic acid molecule is differentially expressed when the amount of one or more of its expression products (e.g., transcript, such as mRNA, and/or protein) is higher or lower in one sample (such as a test sample) as compared to another sample (such as a control). Detecting differential expression can include measuring a change in gene (such as by measuring mRNA) or protein expression.

Enzalutamide: Enzalutamide (e.g., XTANDI®) is an androgen receptor signaling inhibitor that can block binding of androgen to androgen receptors with high affinity and/or inhibit androgen receptor nuclear translocation and binding to DNA. Enzalutamide is used to treat subjects with prostate cancer (such as CPRC). Treatment of prostate cancer with enzalutamide is typically effective for a period of time; however, resistance to enzalutamide can develop. “Enzalutamide resistant” refers to prostate cancer that is not inhibited or treated by enzalutamide therapy, such as with respect to prostate cancer growth or metastasis. In some examples, enzalutamide resistant prostate cancer progresses, for example, with tumor growth or recurrence (such as local recurrence or local or distant metastases). “Enzalutamide sensitive” refers to prostate cancer that is treated or inhibited by treatment (e.g., responds to treatment) with enzalutamide, such as inhibition of prostate cancer growth or metastases.

Expression: Translation of a nucleic acid into a peptide or protein. Peptides or proteins may be expressed and remain intracellular, become a component of the cell surface membrane, or be secreted into the extracellular matrix or medium.

Inhibiting or treating a disease: Inhibiting the full development of a disease or condition, for example, in a subject who is at risk for a disease, such as a subject with cancer, for example, prostate cancer. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. Disease progression refers to a new tumor event, including tumor re-occurrence, and local and distant metastases. A “prophylactic” treatment is a treatment administered to a subject who does not exhibit signs of a disease or exhibits only early signs for the purpose of decreasing the risk of developing pathology.

MYC proto-oncogene (MYC): Myc encodes a nuclear phosphoprotein that is involved in cell cycle progression, apoptosis, and cellular transformation. MYC forms a heterodimer with MAX and binds to the E box DNA consensus sequence and regulates transcription of target genes. Exemplary MYC nucleic acid and proteins include GenBank Accession Nos. NM_002467.6 and NP_002458.2, respectively. Other MYC2 molecules are possible. One of ordinary skill in the art can identify additional MYC nucleic acid and protein sequences, including variants. In some examples, MYC is down-regulated (e.g., expression of MYC mRNA is decreased) in prostate cancer that will respond to enzalutamide, for example as compared to in a prostate cancer that will not respond (e.g., is resistant) to enzalutamide therapy.

NME/NM23 nucleoside diphosphate kinase 2 (NME2): Nucleoside diphosphate kinase is an enzyme that is a hexamer composed of NME1 and NME2 isoforms. NME2 is a transcriptional activator of MYC and binds to both single-stranded guanine and cytosine rich strands in the nuclease hypersensitive III (1) region of the MYC promoter. Exemplary NME2 nucleic acid and proteins include GenBank Accession Nos. NM_002512.4 and NP_002503.1, respectively. Other NME2 molecules are possible. One of ordinary skill in the art can identify additional NME2 nucleic acid and protein sequences, including splice variants. In some examples, NME2 is down-regulated (e.g., expression of NME2 mRNA is decreased) in prostate cancer that will respond to enzalutamide, for example as compared to in a prostate cancer that will not respond (e.g., is resistant) to enzalutamide therapy.

Sample or biological sample: A sample of biological material obtained from a subject, which can include cells, proteins, and/or nucleic acid molecules. Biological samples include all clinical samples useful for detection or analysis of disease, such as cancer, in subjects. Appropriate samples include any conventional biological samples, including clinical samples obtained from a human or veterinary subject. Exemplary samples include, without limitation, cancer or tumor samples (such as from surgery, tissue biopsy, tissue sections, or autopsy), cells, cell lysates, blood smears, cytocentrifuge preparations, cytology smears, bodily fluids (e.g., blood, plasma, serum, saliva, sputum, urine, bronchoalveolar lavage, semen, cerebrospinal fluid (CSF), etc.), or fine-needle aspirates. Samples may be used directly from a subject, or may be processed before analysis (such as concentrated, diluted, purified, such as isolation and/or amplification of nucleic acid molecules in the sample). In a particular example, a sample or biological sample is obtained from a subject having, suspected of having, or at risk of having cancer (such as prostate cancer). In a specific example, the sample is a prostate cancer sample.

Subject: As used herein, the term “subject” refers to a mammal and includes, without limitation, humans, domestic animals (e.g., dogs or cats), farm animals (e.g., cows, horses, or pigs), and laboratory animals (mice, rats, hamsters, guinea pigs, pigs, rabbits, dogs, or monkeys). In one example, the subject treated and/or analyzed with the disclosed methods has cancer, such as prostate cancer. In some examples, the subject responds positively to enzalutamide therapy, such as a subject who does not develop resistance to enzalutamide therapy. In other examples, the subject has or is likely to develop resistance to enzalutamide therapy.

Therapeutically effective amount: The amount of an active ingredient that is sufficient to effect treatment when administered to a mammal in need of such treatment, such as treatment of a cancer (such as prostate cancer). The therapeutically effective amount will vary depending upon the subject and disease condition being treated, the weight and age of the subject, the severity of the disease condition, the manner of administration and the like, which can readily be determined by a prescribing physician.

Treating, treatment, and therapy: Any success or indicia of success in the attenuation or amelioration of an injury, pathology, or condition, including any objective or subjective parameter such as abatement, remission, diminishing of symptoms or making the condition more tolerable to the patient, slowing in the rate of progression, degeneration or decline, making the final point of degeneration less debilitating. The treatment may be assessed by objective or subjective parameters; including the results of a physical examination, neurological examination, or psychiatric evaluations. For example, treatment of a cancer can include decreasing the size, volume, or weight of a cancer, decrease the number, size, volume, or weight of metastases, or combinations thereof.

Tumor, neoplasia, malignancy or cancer: A neoplasm is an abnormal growth of tissue or cells which results from excessive cell division. Neoplastic growth can produce a tumor. The amount of a tumor in an individual is the “tumor burden”, which can be measured as the number, volume, or weight of the tumor. A tumor that does not metastasize is referred to as “benign.” A tumor that invades the surrounding tissue and/or can metastasize is referred to as “malignant.” A “non-cancerous tissue” is a tissue from the same organ wherein the malignant neoplasm formed, but does not have the characteristic pathology of the neoplasm. Generally, noncancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer. Exemplary tumors, such as cancers, that can be analyzed and treated with the disclosed methods include prostate cancers.

### II. Overview

One of the most important oncogenes that has been observed to be overexpressed in almost 60% of CRPC is MYC (Rebello et al., Genes 8:71, 2017). MYC is known to stimulate cell growth and also has been associated with pro-tumorigenic activation in PCa. For example, MYC is known to upregulate EZH2, which is associated with prostate cancer progression. Apart from disease progression, a recent study by Arriaga et al. (Nature Cancer 1:1082-1096, 2020) have demonstrated MYC to be associated with poor response to ARSI in CRPC patients. While studies have shown a tight association of MYC activity with disease progression and response to ARSI, a direct implication of MYC activity in predicting response to Enzalutamide has not been elucidated. Thus to evaluate if MYC-associated mechanisms could serve as a functional biomarker and therapeutic target in Enzalutamide-resistant conditions, disclosed herein is elucidation of a de novo CRPC-specific mechanism-centric regulatory network, which connects biological pathways with their upstream transcriptional regulatory programs. This network was mined with signatures of favorable and poor Enzalutamide response using Partial Least Squares (PLS)-inspired approach. The disclosed analysis nominated MYC pathway and its upstream transcriptional regulatory program NME2 as a mechanism that differentiates favorable and poor response to Enzalutamide.

As disclosed herein, this finding was validated in multiple independent patient cohorts including single cell untreated and Enzalutamide-resistant samples and CRPC patients subjected to adjuvant Enzalutamide treatment. Additionally, the data provided herein confirmed poor predictive ability of MYC and NME2 programs in CRPC patients subjected to adjuvant Abiraterone. Furthermore, the ability to predict patients at risk of Enzalutamide resistance is shown to outperform the predictive ability of markers associated to overall PCa aggressiveness, ARSI resistance, and Enzalutamide resistance. Following identification of NME2 and MYC as a marker of response to Enzalutamide, we further explored the utility of MYC-centric therapeutic targeting by subjecting cells from LNCap and C42B cell lines under Enzalutamide naïve and Enzalutamide resistant conditions to Enzalutamide, and observed that therapeutic targeting of MYC programs reverses Enzalutamide-resistant aggressive phenotype and re-sensitizes LNCaP and C42B Enzalutamide resistant cells to Enzalutamide. We further evaluated the proliferative and metastatic capacity of Enzalutamide resistant cells from C42B cell line using colony formation and migration assay and observed that C42B Enzalutamide resistant cells when subjected to MYC inhibitor, Myc-i975 and Enzalutamide treatment experience significant reduction in both proliferative and metastatic capacity. Finally, apart from direct targeting of MYC, our study also demonstrated that by indirect targeting of MYC via NME2 knockdown in Enzalutamide resistant C42B cells and further treating these cells with Enzalutamide resulted in low metastatic capability, indicating that Enzalutamide resistant cells can recover and become sensitive to Enzalutamide even by indirect targeting of MYC via NME2. Thus, we propose that MYC-associated mechanisms could serve as a biomarker of primary resistance to Enzalutamide aiming to identify patients that are at risk of developing resistance and that should potentially be offered alternative line of treatment. Moreover, our in vitro studies indicate that therapeutic targeting of MYC-associated mechanisms constitutes a valuable primary treatment strategy for these patients and provides a potential secondary rescue therapy for patients that failed Enzalutamide.

### III. Evaluating Expression in a Subject with Cancer

Provided herein are methods of identifying a subject with prostate cancer (such as a human or veterinary subject) who will respond to enzalutamide therapy. In particular examples, the methods can determine with high accuracy whether a subject is likely to respond to enzalutamide therapy. Also provided are methods for treating a subject who is likely to respond to enzalutamide, for example by administering enzalutamide to the subject.

The methods herein can be used to treat subjects with prostate cancer with enzalutamide or identify subjects who respond to enzalutamide. It is helpful to determine whether or not a subject is responsive to enzalutamide because many subjects with prostate cancer develop enzalutamide resistance. Hence, using the results of the disclosed methods allows subjects to be administered an effective therapy, such as enzalutamide or an alternative treatment, such as abiraterone.

Examples of methods for treating a subject with prostate cancer or identifying a subject with prostate cancer who responds positively to enzalutamide therapy are disclosed herein (such as a subject with prostate cancer that will be treated by the enzalutamide therapy (such as a reduction in the size or metastasis of a tumor), and/or who does not develop resistance to enzalutamide therapy). In some examples, the methods include measuring expression of one or more enzalutamide resistance-related molecules from one or more enzalutamide resistance-related molecular pathways and/or transcriptional regulatory programs in a sample obtained from a subject (such as a prostate cancer sample). A variety of molecules from the one or more enzalutamide resistance-related molecular pathways and/or transcriptional regulatory programs can be measured. Further, the methods can include measuring any number of molecules. For example, at least about two, at least about three, at least about four, at least about five, at least about six, at least about seven, at least about eight, at least about nine, at least about 10, at least about 15, at least about 20, at least about 25, at least about 50, or about 2-5, about 2 to 7, about 2-10, about 1-25, about 10-50, molecules can be measured. In some examples, molecules from at least about one, at least about two, at least about three, at least about four, at least about five, at least about six, or at least about seven enzalutamide resistance-related molecular pathways and/or transcriptional regulatory programs can be measured.

The methods herein can further include comparing the expression of enzalutamide resistance-related molecules from one or more enzalutamide resistance-related molecular pathways and/or transcriptional regulatory programs measured in a sample obtained from a subject. In some examples, the measured expression is similar to the expression of enzalutamide resistance-related molecules in a control representing expression for the enzalutamide resistance-related molecules expected in a sample from a subject who positively responds to enzalutamide therapy (such as a subject with prostate cancer that will be treated by enzalutamide therapy (such as a reduction in the size or metastasis of a tumor), and/or who does not develop resistance to enzalutamide therapy). Where such similar expression is measured, the subject can be identified as a subject who responds positively to enzalutamide therapy. Where such similar expression is measured, the subject can be identified as a subject who responds positively to enzalutamide therapy. Conversely, where similar expression is not present, the subject can be identified as a subject who will not respond positively to enzalutamide therapy (such as a subject with cancer that will not be treated by the enzalutamide therapy (such as a reduction in the size or metastasis of a tumor), and/or who does develop resistance to enzalutamide therapy).

In some examples, the measured expression of enzalutamide resistance-related molecules differs from the expression\ of the enzalutamide resistance-related molecules in a control representing expression for the enzalutamide resistance-related molecules expected in a sample from a subject who does not positively respond to enzalutamide therapy (such as a subject with cancer that will not be treated by the enzalutamide therapy (such as a reduction in the size or metastasis of a tumor), and/or who does develop resistance to enzalutamide therapy). Where such differential expression is measured, the subject can be identified as a subject who responds positively to enzalutamide therapy. Conversely, where differential expression is not measured, the subject can be identified as a subject who does not respond positively to enzalutamide therapy (such as a subject with cancer that will not be treated by the enzalutamide therapy (such as a reduction in the size or metastasis of a tumor), and/or who does develop resistance to enzalutamide therapy). In some examples, the methods include administering enzalutamide therapy to a subject identified as one who will respond positively to enzalutamide therapy (such as a subject with prostate cancer that will be treated by the enzalutamide therapy (such as a reduction in the size or metastasis of a tumor), and/or who does not develop resistance to enzalutamide therapy), thereby treating the subject. In other examples, the methods include administering other types of cancer therapy (such as surgery, radiation therapy, targeted therapy, immunotherapy, or palliative care) to a subject identified as one who will not respond positively to enzalutamide therapy, thereby treating the subject. In some examples, the methods include administering an androgen receptor signaling inhibitor that is not enzalutamide (such as abiraterone) to the subject identified as one who will not respond positively to enzalutamide therapy, thereby treating the subject.

### IV. Detecting Expression of Enzalutamide Resistance-Related Molecules

As described herein, expression of any enzalutamide resistance-related molecules or combination thereof disclosed herein (such as enzalutamide resistance-related molecules one or more enzalutamide resistance-related molecular pathways and/or transcriptional regulatory programs can be detected alone or in combination using a variety of methods. In some implementations, expression of one or more (such as 1, 2, 3, 4, 5, 10, 15, 20, 25, 30, 35, 40, 45, 50, 55, or all) of MYC, SLC19A1, MRTO4, TMEM97, RRP9, PES1, TFB2M, EXOSC5, IPO4, NDUFAF4, NOC4L, SRM, PA2G4, GNL3, NOLC1, WDR43, RABEPK, NOP16, TBRG4, DDX18, NIP7, WDR74, BYSL, HSPD1, PLK4, NOP2, PPAN, NOP56, RCL1, NPM1, AIMP2, RRP12, PPRC1, TCOF1, MCM5, HK2, CBX3, PLK1, PHB, MCM4, CDK4, DUSP2, MYBBP1A, UTP20, PRMT3, FARSA, MAP3K6, LAS1L, PUS1, HSPE1, SLC29A2, DCTPP1, SUPV3L1, SORD, IMP4, GRWD1, UNG, MPHOSPH10, and NME2 are detected. Expression of nucleic acid molecules (e.g., mRNA, cDNA) or protein is contemplated herein.

1. Methods for Detecting mRNA Expression

Gene expression can be evaluated by detecting mRNA encoding the gene of interest. Thus, the disclosed methods can include evaluating mRNA encoding enzalutamide resistance-related molecules from one or more enzalutamide resistance-related molecular pathways and/or transcriptional regulatory programs (such as one or more of MYC, SLC19A1, MRTO4, TMEM97, RRP9, PES1, TFB2M, EXOSC5, IPO4, NDUFAF4, NOC4L, SRM, PA2G4, GNL3, NOLC1, WDR43, RABEPK, NOP16, TBRG4, DDX18, NIP7, WDR74, BYSL, HSPD1, PLK4, NOP2, PPAN, NOP56, RCL1, NPM1, AIMP2, RRP12, PPRC1, TCOF1, MCM5, HK2, CBX3, PLK1, PHB, MCM4, CDK4, DUSP2, MYBBP1A, UTP20, PRMT3, FARSA, MAP3K6, LAS1L, PUS1, HSPE1, SLC29A2, DCTPP1, SUPV3L1, SORD, IMP4, GRWD1, UNG, MPHOSPH10, and NME2). In some examples, mRNA expression is quantified.

RNA can be isolated from a sample (such as a prostate cancer sample) from a subject, for example using commercially available kits, such as those from QIAGEN®. General methods for mRNA extraction are disclosed in, for example, including Ausubel et al., Current Protocols of Molecular Biology, John Wiley and Sons (1997). RNA can be extracted from paraffin embedded tissues (e.g., see Rupp and Locker, Lab Invest. 56: A67 (1987), and De Andres et al., BioTechniques 18:42044 (1995)). Total RNA from cells in culture (such as those obtained from a subject) can be isolated using QIAGIN® RNeasy mini-columns. Other commercially available RNA isolation kits include MASTERPURE®. Complete DNA and RNA Purification Kit (EPICENTRE® Madison, Wis.), and Paraffin Block RNA Isolation Kit (Ambion, Inc.). Total RNA from tissue samples can be isolated using RNA Stat-60 (Tel-Test). RNA prepared from tumor or other biological sample can be isolated, for example, by cesium chloride density gradient centrifugation.

Methods of gene expression profiling include methods based on hybridization analysis of polynucleotides, methods based on sequencing of polynucleotides, and proteomics-based methods. In some examples, mRNA expression in a sample is quantified using northern blotting or in situ hybridization (Parker & Barnes, Methods in Molecular Biology 106:247-283, 1999); RNAse protection assays (Hod, Biotechniques 13:852-4, 1992); or PCR-based methods, such as reverse transcription polymerase chain reaction (RT-PCR) (Weis et al., Trends in Genetics 8:263-4, 1992). Alternatively, antibodies can be employed that can recognize specific duplexes, including DNA duplexes, RNA duplexes, and DNA-RNA hybrid duplexes or DNA-protein duplexes. Representative methods for sequencing-based gene expression analysis include RNA sequencing (RNA-seq), single cell RNA sequencing (scRNA-seq), Serial Analysis of Gene Expression (SAGE), and gene expression analysis by massively parallel signature sequencing (MPSS).

In one example, RT-PCR can be used. Generally, the first step in gene expression profiling by RT-PCR is the reverse transcription of the RNA template into cDNA, followed by its exponential amplification in a PCR reaction. Two commonly used reverse transcriptases are avian myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase. TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

To minimize errors and the effect of sample-to-sample variation, RT-PCR can be performed using an internal standard. The ideal internal standard is expressed at a constant level among different tissues, and is unaffected by the experimental treatment. RNAs commonly used to normalize patterns of gene expression are mRNAs for the housekeeping genes glyceraldehyde-3-phosphate-dehydrogenase (GAPDH), beta-actin, tubulin, and 18S ribosomal RNA.

A variation of RT-PCR is real time quantitative RT-PCR, which measures PCR product accumulation through a dual-labeled fluorogenic probe (e.g., TAQMAN® probe). Real time PCR is compatible both with quantitative competitive PCR, where internal competitor for each target sequence is used for normalization, and with quantitative comparative PCR using a normalization gene contained within the sample, or a housekeeping gene for RT-PCR (see Held et al., Genome Research 6:986 994, 1996). Quantitative PCR is also described in U.S. Pat. No. 5,538,848. Related probes and quantitative amplification procedures are described in U.S. Pat. Nos. 5,716,784 and 5,723,591. Instruments for carrying out quantitative PCR in microtiter plates are commercially available.

The steps of a representative protocol for quantifying gene expression using fixed, paraffin-embedded tissues as the RNA source, including mRNA isolation, purification, primer extension and amplification are given in various publications (see Godfrey et al., J. Mol. Diag. 2:84 91, 2000; Specht et al., Am. J. Pathol. 158:419-29, 2001). Briefly, a representative process starts with cutting about 10 μm thick sections of paraffin-embedded tumor tissue samples or adjacent non-cancerous tissue. The RNA is then extracted, and protein and DNA are removed. Alternatively, RNA is located directly from a tumor sample or other tissue sample. After analysis of the RNA concentration, RNA repair and/or amplification steps can be included, if necessary, and RNA is reverse transcribed using gene specific promoters followed by RT-PCR.

Primers used for amplification of the mRNA(s) are selected so as to amplify a unique segment of the gene of interest, such as mRNA encoding enzalutamide resistance-related molecules from one or more enzalutamide resistance-related molecular pathways and/or transcriptional regulatory programs (such as one or more of MYC, SLC19A1, MRTO4, TMEM97, RRP9, PES1, TFB2M, EXOSC5, IPO4, NDUFAF4, NOC4L, SRM, PA2G4, GNL3, NOLC1, WDR43, RABEPK, NOP16, TBRG4, DDX18, NIP7, WDR74, BYSL, HSPD1, PLK4, NOP2, PPAN, NOP56, RCL1, NPM1, AIMP2, RRP12, PPRC1, TCOF1, MCM5, HK2, CBX3, PLK1, PHB, MCM4, CDK4, DUSP2, MYBBP1A, UTP20, PRMT3, FARSA, MAP3K6, LAS1L, PUS1, HSPE1, SLC29A2, DCTPP1, SUPV3L1, SORD, IMP4, GRWD1, UNG, MPHOSPH10, and NME2). In some implementations, expression of other genes is also detected. Primers that can be used to amplify mRNA encoding enzalutamide resistance-related molecules from one or more enzalutamide resistance-related molecular pathways and/or transcriptional regulatory programs (such as one or more of MYC, SLC19A1, MRTO4, TMEM97, RRP9, PES1, TFB2M, EXOSC5, IPO4, NDUFAF4, NOC4L, SRM, PA2G4, GNL3, NOLC1, WDR43, RABEPK, NOP16, TBRG4, DDX18, NIP7, WDR74, BYSL, HSPD1, PLK4, NOP2, PPAN, NOP56, RCL1, NPM1, AIMP2, RRP12, PPRC1, TCOF1, MCM5, HK2, CBX3, PLK1, PHB, MCM4, CDK4, DUSP2, MYBBP1A, UTP20, PRMT3, FARSA, MAP3K6, LAS1L, PUS1, HSPE1, SLC29A2, DCTPP1, SUPV3L1, SORD, IMP4, GRWD1, UNG, MPHOSPH10, and NME2) are commercially available or can be designed and synthesized. In some examples, the primers specifically hybridize to a promoter or promoter region of an enzalutamide resistance-related molecule from one or more enzalutamide resistance-related molecular pathways and/or transcriptional regulatory programs (such as one or more of MYC, SLC19A1, MRTO4, TMEM97, RRP9, PES1, TFB2M, EXOSC5, IPO4, NDUFAF4, NOC4L, SRM, PA2G4, GNL3, NOLC1, WDR43, RABEPK, NOP16, TBRG4, DDX18, NIP7, WDR74, BYSL, HSPD1, PLK4, NOP2, PPAN, NOP56, RCL1, NPM1, AIMP2, RRP12, PPRC1, TCOF1, MCM5, HK2, CBX3, PLK1, PHB, MCM4, CDK4, DUSP2, MYBBP1A, UTP20, PRMT3, FARSA, MAP3K6, LAS1L, PUS1, HSPE1, SLC29A2, DCTPP1, SUPV3L1, SORD, IMP4, GRWD1, UNG, MPHOSPH10, and NME2).

An alternative quantitative nucleic acid amplification procedure is described in U.S. Pat. No. 5,219,727. In this procedure, the amount of a target sequence in a sample is determined by simultaneously amplifying the target sequence and an internal standard nucleic acid segment. The amount of amplified DNA from each segment is determined and compared to a standard curve to determine the amount of the target nucleic acid segment that was present in the sample prior to amplification.

In some implementations of this method, the expression of a “housekeeping” gene or “internal control” can also be evaluated. These terms include any constitutively or globally expressed gene whose presence enables an assessment of mRNA levels provided herein. Such an assessment includes a determination of the overall constitutive level of gene transcription and a control for variations in RNA recovery. Exemplary housekeeping genes include β-actin and tubulin.

In some examples, gene expression is identified or confirmed using a microarray technique. Thus, the expression profile can be measured in either fresh or paraffin-embedded tumor tissue, using microarray technology. In this method, nucleic acid sequences (including cDNAs and oligonucleotides) encoding enzalutamide resistance-related molecules from one or more enzalutamide resistance-related molecular pathways and/or transcriptional regulatory programs (such as one or more of MYC, SLC19A1, MRTO4, TMEM97, RRP9, PES1, TFB2M, EXOSC5, IPO4, NDUFAF4, NOC4L, SRM, PA2G4, GNL3, NOLC1, WDR43, RABEPK, NOP16, TBRG4, DDX18, NIP7, WDR74, BYSL, HSPD1, PLK4, NOP2, PPAN, NOP56, RCL1, NPM1, AIMP2, RRP12, PPRC1, TCOF1, MCM5, HK2, CBX3, PLK1, PHB, MCM4, CDK4, DUSP2, MYBBP1A, UTP20, PRMT3, FARSA, MAP3K6, LAS1L, PUS1, HSPE1, SLC29A2, DCTPP1, SUPV3L1, SORD, IMP4, GRWD1, UNG, MPHOSPH10, and NME2) are plated, or arrayed, on a microchip substrate. The arrayed sequences are then hybridized with specific DNA probes from cells or tissues of interest. Just as in the RT-PCR method, the source of mRNA typically is total RNA isolated from human tumors, and optionally from corresponding noncancerous tissue and normal tissues or cell lines.

In a specific implementation of the microarray technique, PCR amplified inserts of cDNA clones are applied to a substrate in a dense array. At least probes specific for nucleotide sequences mRNA encoding enzalutamide resistance-related molecules from one or more enzalutamide resistance-related molecular pathways and/or transcriptional regulatory programs (such as one or more of MYC, SLC19A1, MRTO4, TMEM97, RRP9, PES1, TFB2M, EXOSC5, IPO4, NDUFAF4, NOC4L, SRM, PA2G4, GNL3, NOLC1, WDR43, RABEPK, NOP16, TBRG4, DDX18, NIP7, WDR74, BYSL, HSPD1, PLK4, NOP2, PPAN, NOP56, RCL1, NPM1, AIMP2, RRP12, PPRC1, TCOF1, MCM5, HK2, CBX3, PLK1, PHB, MCM4, CDK4, DUSP2, MYBBP1A, UTP20, PRMT3, FARSA, MAP3K6, LAS1L, PUS1, HSPE1, SLC29A2, DCTPP1, SUPV3L1, SORD, IMP4, GRWD1, UNG, MPHOSPH10, and NME2) (and, in some examples, one or more housekeeping genes) are applied to the substrate, and the array can consist essentially of, or consist of these sequences. The microarrayed nucleic acids are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for enzalutamide resistance-related molecules from one or more enzalutamide resistance-related molecular pathways and/or transcriptional regulatory programs (such as one or more of MYC, SLC19A1, MRTO4, TMEM97, RRP9, PES1, TFB2M, EXOSC5, IPO4, NDUFAF4, NOC4L, SRM, PA2G4, GNL3, NOLC1, WDR43, RABEPK, NOP16, TBRG4, DDX18, NIP7, WDR74, BYSL, HSPD1, PLK4, NOP2, PPAN, NOP56, RCL1, NPM1, AIMP2, RRP12, PPRC1, TCOF1, MCM5, HK2, CBX3, PLK1, PHB, MCM4, CDK4, DUSP2, MYBBP1A, UTP20, PRMT3, FARSA, MAP3K6, LAS1L, PUS1, HSPE1, SLC29A2, DCTPP1, SUPV3L1, SORD, IMP4, GRWD1, UNG, MPHOSPH10, and NME2). Microarray analysis can be performed by commercially available equipment, following manufacturer's protocols.

Serial analysis of gene expression (SAGE) allows the simultaneous and quantitative analysis of a large number of gene transcripts, without the need of providing an individual hybridization probe for each transcript. First, a short sequence tag (about 10-14 base pairs) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag (see, for example, Velculescu et al., Science 270:484-7, 1995; and Velculescu et al., Cell 88:243-51, 1997).

In situ hybridization (ISH) is another method for detecting and comparing expression of enzalutamide resistance-related molecules from one or more enzalutamide resistance-related molecular pathways and/or transcriptional regulatory programs (such as one or more of MYC, SLC19A1, MRTO4, TMEM97, RRP9, PES1, TFB2M, EXOSC5, IPO4, NDUFAF4, NOC4L, SRM, PA2G4, GNL3, NOLC1, WDR43, RABEPK, NOP16, TBRG4, DDX18, NIP7, WDR74, BYSL, HSPD1, PLK4, NOP2, PPAN, NOP56, RCL1, NPM1, AIMP2, RRP12, PPRC1, TCOF1, MCM5, HK2, CBX3, PLK1, PHB, MCM4, CDK4, DUSP2, MYBBP1A, UTP20, PRMT3, FARSA, MAP3K6, LAS1L, PUS1, HSPE1, SLC29A2, DCTPP1, SUPV3L1, SORD, IMP4, GRWD1, UNG, MPHOSPH10, and NME2). ISH applies and extrapolates the technology of nucleic acid hybridization to the single cell level, and, in combination with the art of cytochemistry, immunocytochemistry and immunohistochemistry, permits the maintenance of morphology and the identification of cellular markers to be maintained and identified, and allows the localization of sequences to specific cells within populations, such as tissues and blood samples. ISH is a type of hybridization that uses a complementary nucleic acid to localize one or more specific nucleic acid sequences in a portion or section of tissue (in situ), or, if the tissue is small enough, in the entire tissue (whole mount ISH). RNA ISH can be used to assay expression patterns in a tissue, such as the expression of enzalutamide resistance-related molecules from one or more enzalutamide resistance-related molecular pathways and/or transcriptional regulatory programs (such as one or more of MYC, SLC19A1, MRTO4, TMEM97, RRP9, PES1, TFB2M, EXOSC5, IPO4, NDUFAF4, NOC4L, SRM, PA2G4, GNL3, NOLC1, WDR43, RABEPK, NOP16, TBRG4, DDX18, NIP7, WDR74, BYSL, HSPD1, PLK4, NOP2, PPAN, NOP56, RCL1, NPM1, AIMP2, RRP12, PPRC1, TCOF1, MCM5, HK2, CBX3, PLK1, PHB, MCM4, CDK4, DUSP2, MYBBP1A, UTP20, PRMT3, FARSA, MAP3K6, LAS1L, PUS1, HSPE1, SLC29A2, DCTPP1, SUPV3L1, SORD, IMP4, GRWD1, UNG, MPHOSPH10, and NME2). In situ PCR is the PCR-based amplification of the target nucleic acid sequences prior to ISH. For detection of RNA, an intracellular reverse transcription step is introduced to generate complementary DNA from RNA templates prior to in situ PCR. This enables detection of low copy RNA sequences.

Gene expression can also be detected and quantitated using the nCounter® technology developed by NanoString (Seattle, WA; see, for example, U.S. Pat. Nos. 7,473,767; 7,919,237; and 9,371,563, which are herein incorporated by reference in their entireties). The nCounter® analysis system utilizes a digital color-coded barcode technology that is based on direct multiplexed measurement of gene expression. The technology uses molecular “barcodes” and single molecule imaging to detect and count hundreds of unique transcripts in a single reaction. Each color-coded barcode is attached to a single target-specific probe corresponding to a gene of interest (such as a TACE-response gene). Mixed together with controls, they form a multiplexed CodeSet.

Each color-coded barcode represents a single target molecule. Barcodes hybridize directly to target molecules and can be individually counted without the need for amplification. The method includes three steps: (1) hybridization; (2) purification and immobilization; and (3) counting. The technology employs two approximately 50 base probes per mRNA that hybridize in solution. The reporter probe carries the signal; the capture probe allows the complex to be immobilized for data collection. After hybridization, the excess probes are removed and the probe/target complexes are aligned and immobilized in the nCounter® cartridge. Sample cartridges are placed in the digital analyzer for data collection. Color codes on the surface of the cartridge are counted and tabulated for each target molecule. This method is described in, for example, U.S. Pat. No. 7,919,237; and U.S. Patent Application Publication Nos. 20100015607; 20100112710; 20130017971. Information on this technology can also be found on the company's website (nanostring.com).

Gene expression can also be detected and quantitated using RNA sequencing (RNA-seq), such as single cell RNA-seq (scRNA-seq) (see Stark, et al., Nat Rev Genet. 2019; 20, 631-656; Haque, et al., Genome Med. 2017; 9 (75)). RNA-seq is most frequently used for analyzing differential gene expression between samples. In traditional RNA-seq analyses, the process of analyzing differential gene expression via RNA-seq begins with RNA extraction (such as from a tumor sample, such as a prostate cancer sample), followed by mRNA enrichment or ribosomal RNA depletion. cDNA is then synthesized, and an adaptor-ligated sequencing library is prepared. The library is sequenced to a read depth of, for example, 10-30 million reads per sample on a high-throughput platform (such as an Illumina platform). The sequencing reads (most often in the form of FASTQ files) are computationally aligned and/or assembled to a transcriptome. The reads are most often mapped to a known transcriptome or annotated genome, matching each read to one or more genomic coordinates. This process is often accomplished using alignment tools such as STAR, TopHat, or HISAT, which each rely on a reference genome. If no genome annotation containing known exon boundaries is available (such as if a reference genome annotation is missing or is incomplete), or if reads are to be associated with transcripts rather than genes, aligned reads can be used in a transcriptome assembly step using tools such as StringTie or SOAPdenovo-Trans. Tools such as Sailfish, Kallisto, and Salmon can associate sequencing reads directly with transcripts, without the need for a separate quantification step. Next, reads that have been mapped to transcriptomic or genomic locations are quantified using tools such as RSEM, CuffLinks, MMSeq, or HTSeq, or the alignment-free direct quantification tools Sailfish, Kallisto, or Salmon. Quantification results are often combined into an expression matrix, with one row for each expression feature (gene or transcript) and one column for each sample, with values being read counts or estimated abundances. Samples are then filtered and normalized to account for differences in expression patterns, read depth, and/or technical biases. Significant changes in expression of individual genes and/or transcripts between sample groups are then statistically modeled using one or more of various tools and computational methods.

In a scRNA-seq analysis, a tissue sample (such as a prostate cancer tissue sample) is dissociated, single cells are separated, and RNA from each individual cell is converted to cDNA (and can be labelled during reverse transcription) and then amplified (typically using PCR) for sequencing. The synthesized cDNA is used as the input for library preparation. Amplified nucleic acids can also be labelled with barcodes (such as using single-cell combinatorial indexing RNA sequencing or split-pool ligation-based transcriptome sequencing). Tissue dissociation may be accomplished using methods known in the art, such as mechanical disaggregation and/or enzymatic dissociation, such as enzymatic dissociation using collagenase and/or DNase. Similarly, single cells can be separated using known methods, such as flow-cytometry, wherein cells can be flow-sorted directly into micro-plates containing lysis buffer. Individual cells can also be captured in microfluidic chips or loaded into nano-well devices (e.g., by Poisson distribution), isolated, and merged into droplets (containing reagents) via droplet-microfluidic isolation (such as Drop-Seq or InDrop). Isolated single cells are then lysed such that RNA can be released for cDNA synthesis.

### 2. Arrays for Profiling Gene Expression

In particular implementations, arrays (such as a solid support) are provided that can be used to evaluate gene expression, for example to determine if a patient with prostate cancer will respond to enzalutamide therapy. Such arrays can include a set of specific binding agents (such as nucleic acid probes and/or primers specific for enzalutamide resistance-related molecules from one or more enzalutamide resistance-related molecular pathways and/or transcriptional regulatory programs (such as one or more of MYC, SLC19A1, MRTO4, TMEM97, RRP9, PES1, TFB2M, EXOSC5, IPO4, NDUFAF4, NOC4L, SRM, PA2G4, GNL3, NOLC1, WDR43, RABEPK, NOP16, TBRG4, DDX18, NIP7, WDR74, BYSL, HSPD1, PLK4, NOP2, PPAN, NOP56, RCL1, NPM1, AIMP2, RRP12, PPRC1, TCOF1, MCM5, HK2, CBX3, PLK1, PHB, MCM4, CDK4, DUSP2, MYBBP1A, UTP20, PRMT3, FARSA, MAP3K6, LAS1L, PUS1, HSPE1, SLC29A2, DCTPP1, SUPV3L1, SORD, IMP4, GRWD1, UNG, MPHOSPH10, and NME2) provided herein. When describing an array that consists essentially of probes or primers specific for enzalutamide resistance-related molecules from one or more enzalutamide resistance-related molecular pathways and/or transcriptional regulatory programs (such as one or more of MYC, SLC19A1, MRTO4, TMEM97, RRP9, PES1, TFB2M, EXOSC5, IPO4, NDUFAF4, NOC4L, SRM, PA2G4, GNL3, NOLC1, WDR43, RABEPK, NOP16, TBRG4, DDX18, NIP7, WDR74, BYSL, HSPD1, PLK4, NOP2, PPAN, NOP56, RCL1, NPM1, AIMP2, RRP12, PPRC1, TCOF1, MCM5, HK2, CBX3, PLK1, PHB, MCM4, CDK4, DUSP2, MYBBP1A, UTP20, PRMT3, FARSA, MAP3K6, LAS1L, PUS1, HSPE1, SLC29A2, DCTPP1, SUPV3L1, SORD, IMP4, GRWD1, UNG, MPHOSPH10, and NME2), such an array includes probes or primers specific for the gene or genes, and can further include control probes or primers, such as 1-10 control probes or primers (for example to confirm the incubation conditions are sufficient). In some examples, the array may further comprise additional, such as 1, 2, 3, 4 or 5 additional probes for other genes. In some examples, the array includes 1-10 housekeeping-specific probes or primers. In one example, an array is a multi-well plate (e.g., 98 or 364 well plate).

In one example, the array includes, consists essentially of, or consists of probes or primers (such as an oligonucleotide or antibody) that can recognize enzalutamide resistance-related molecules from one or more enzalutamide resistance-related molecular pathways and/or transcriptional regulatory programs (such as one or more of MYC, SLC19A1, MRTO4, TMEM97, RRP9, PES1, TFB2M, EXOSC5, IPO4, NDUFAF4, NOC4L, SRM, PA2G4, GNL3, NOLC1, WDR43, RABEPK, NOP16, TBRG4, DDX18, NIP7, WDR74, BYSL, HSPD1, PLK4, NOP2, PPAN, NOP56, RCL1, NPM1, AIMP2, RRP12, PPRC1, TCOF1, MCM5, HK2, CBX3, PLK1, PHB, MCM4, CDK4, DUSP2, MYBBP1A, UTP20, PRMT3, FARSA, MAP3K6, LAS1L, PUS1, HSPE1, SLC29A2, DCTPP1, SUPV3L1, SORD, IMP4, GRWD1, UNG, MPHOSPH10, and NME2) (and, in some examples, also 1-10 housekeeping genes). The oligonucleotide probes or primers can further include one or more detectable labels, to permit detection of hybridization signals between the probe and target sequence (such as enzalutamide resistance-related molecules from one or more enzalutamide resistance-related molecular pathways and/or transcriptional regulatory programs (such as one or more of MYC, SLC19A1, MRTO4, TMEM97, RRP9, PES1, TFB2M, EXOSC5, IPO4, NDUFAF4, NOC4L, SRM, PA2G4, GNL3, NOLC1, WDR43, RABEPK, NOP16, TBRG4, DDX18, NIP7, WDR74, BYSL, HSPD1, PLK4, NOP2, PPAN, NOP56, RCL1, NPM1, AIMP2, RRP12, PPRC1, TCOF1, MCM5, HK2, CBX3, PLK1, PHB, MCM4, CDK4, DUSP2, MYBBP1A, UTP20, PRMT3, FARSA, MAP3K6, LAS1L, PUS1, HSPE1, SLC29A2, DCTPP1, SUPV3L1, SORD, IMP4, GRWD1, UNG, MPHOSPH10, and NME2)).

The solid support of the array can be formed from an organic polymer. Suitable materials for the solid support include, but are not limited to: polypropylene, polyethylene, polybutylene, polyisobutylene, polybutadiene, polyisoprene, polyvinylpyrrolidine, polytetrafluroethylene, polyvinylidene difluoride, polyfluoroethylene-propylene, polyethylenevinyl alcohol, polymethylpentene, polycholorotrifluoroethylene, polysulfornes, hydroxylated biaxially oriented polypropylene, aminated biaxially oriented polypropylene, thiolated biaxially oriented polypropylene, ethyleneacrylic acid, thylene methacrylic acid, and blends of copolymers thereof (see U.S. Pat. No. 5,985,567).

In one example, the solid support surface is polypropylene. In another example, a surface activated organic polymer is used as the solid support surface. One example of a surface activated organic polymer is a polypropylene material aminated via radio frequency plasma discharge. Such materials are easily utilized for the attachment of nucleotide molecules. The amine groups on the activated organic polymers are reactive with nucleotide molecules such that the nucleotide molecules can be bound to the polymers. Other reactive groups can also be used, such as carboxylated, hydroxylated, thiolated, or active ester groups.

A wide variety of array formats can be employed. One example includes a linear array of oligonucleotide bands, generally referred to in the art as a dipstick. Another suitable format includes a two-dimensional pattern of discrete cells (such as 4096 squares in a 64 by 64 array). Other array formats including, but not limited to slot (rectangular) and circular arrays are equally suitable for use. In some examples, the array is a multi-well plate. In one example, the array is formed on a polymer medium, which is a thread, membrane or film. An example of an organic polymer medium is a polypropylene sheet having a thickness on the order of about 1 mil. (0.001 inch) to about 20 mil., although the thickness of the film is not critical and can be varied over a fairly broad range. The array can include biaxially oriented polypropylene (BOPP) films, which in addition to their durability, exhibit a low background fluorescence.

The array formats can be included in a variety of different types of formats. A “format” includes any format to which probes, primers or antibodies can be affixed, such as microtiter plates (e.g., multi-well plates), test tubes, inorganic sheets, dipsticks, and the like. For example, when the solid support is a polypropylene thread, one or more polypropylene threads can be affixed to a plastic dipstick-type device; polypropylene membranes can be affixed to glass slides.

The arrays of can be prepared by a variety of approaches. In one example, oligonucleotide or protein sequences are synthesized separately and then attached to a solid support (see U.S. Pat. No. 6,013,789). In another example, sequences are synthesized directly onto the support to provide the desired array (see U.S. Pat. No. 5,554,501). Suitable methods for covalently coupling oligonucleotides and proteins to a solid support and for directly synthesizing the oligonucleotides or proteins onto the support are describe in Matson et al., Anal. Biochem. 217:306-10, 1994. In one example, the oligonucleotides are synthesized onto the support using chemical techniques for preparing oligonucleotides on solid supports (such as see PCT applications WO 85/01051 and WO 89/10977, or U.S. Pat. No. 5,554,501).

The oligonucleotides can be bound to the polypropylene support by either the 3′ end of the oligonucleotide or by the 5′ end of the oligonucleotide. In one example, the oligonucleotides are bound to the solid support by the 3′ end. In general, the internal complementarity of an oligonucleotide probe in the region of the 3′ end and the 5′ end determines binding to the support.

In particular examples, the oligonucleotide probes on the array include one or more labels, that permit detection of oligonucleotide probe: target sequence hybridization complexes.

### 3. Detecting Protein Expression

In some examples, expression of enzalutamide resistance-related proteins from one or more enzalutamide resistance-related molecular pathways and/or transcriptional regulatory programs (such as one or more of MYC, SLC19A1, MRTO4, TMEM97, RRP9, PES1, TFB2M, EXOSC5, IPO4, NDUFAF4, NOC4L, SRM, PA2G4, GNL3, NOLC1, WDR43, RABEPK, NOP16, TBRG4, DDX18, NIP7, WDR74, BYSL, HSPD1, PLK4, NOP2, PPAN, NOP56, RCL1, NPM1, AIMP2, RRP12, PPRC1, TCOF1, MCM5, HK2, CBX3, PLK1, PHB, MCM4, CDK4, DUSP2, MYBBP1A, UTP20, PRMT3, FARSA, MAP3K6, LAS1L, PUS1, HSPE1, SLC29A2, DCTPP1, SUPV3L1, SORD, IMP4, GRWD1, UNG, MPHOSPH10, and NME2) is analyzed. Suitable biological samples include samples containing protein obtained from a cancer (such as a prostate cancer) of a subject. An alteration in the amount of enzalutamide resistance-related proteins from one or more enzalutamide resistance-related molecular pathways and/or transcriptional regulatory programs (such as one or more of MYC, SLC19A1, MRTO4, TMEM97, RRP9, PES1, TFB2M, EXOSC5, IPO4, NDUFAF4, NOC4L, SRM, PA2G4, GNL3, NOLC1, WDR43, RABEPK, NOP16, TBRG4, DDX18, NIP7, WDR74, BYSL, HSPD1, PLK4, NOP2, PPAN, NOP56, RCL1, NPM1, AIMP2, RRP12, PPRC1, TCOF1, MCM5, HK2, CBX3, PLK1, PHB, MCM4, CDK4, DUSP2, MYBBP1A, UTP20, PRMT3, FARSA, MAP3K6, LAS1L, PUS1, HSPE1, SLC29A2, DCTPP1, SUPV3L1, SORD, IMP4, GRWD1, UNG, MPHOSPH10, and NME2) in a tumor (such as a prostate tumor) from the subject relative to a control, such as an increase or decrease in protein expression, indicates whether the prostate cancer will respond to enzalutamide therapy, as described herein.

Antibodies specific for enzalutamide resistance-related proteins from one or more enzalutamide resistance-related molecular pathways and/or transcriptional regulatory programs (such as one or more of MYC, SLC19A1, MRTO4, TMEM97, RRP9, PES1, TFB2M, EXOSC5, IPO4, NDUFAF4, NOC4L, SRM, PA2G4, GNL3, NOLC1, WDR43, RABEPK, NOP16, TBRG4, DDX18, NIP7, WDR74, BYSL, HSPD1, PLK4, NOP2, PPAN, NOP56, RCL1, NPM1, AIMP2, RRP12, PPRC1, TCOF1, MCM5, HK2, CBX3, PLK1, PHB, MCM4, CDK4, DUSP2, MYBBP1A, UTP20, PRMT3, FARSA, MAP3K6, LAS1L, PUS1, HSPE1, SLC29A2, DCTPP1, SUPV3L1, SORD, IMP4, GRWD1, UNG, MPHOSPH10, and NME2) can be used for protein detection and quantification, for example using an immunoassay method, such as those presented in Harlow and Lane (Antibodies, A Laboratory Manual, CSHL, New York, 1988).

Exemplary immunoassay formats include ELISA, Western blot, and RIA assays. Thus, protein levels of enzalutamide resistance-related proteins from one or more enzalutamide resistance-related molecular pathways and/or transcriptional regulatory programs (such as one or more of MYC, SLC19A1, MRTO4, TMEM97, RRP9, PES1, TFB2M, EXOSC5, IPO4, NDUFAF4, NOC4L, SRM, PA2G4, GNL3, NOLC1, WDR43, RABEPK, NOP16, TBRG4, DDX18, NIP7, WDR74, BYSL, HSPD1, PLK4, NOP2, PPAN, NOP56, RCL1, NPM1, AIMP2, RRP12, PPRC1, TCOF1, MCM5, HK2, CBX3, PLK1, PHB, MCM4, CDK4, DUSP2, MYBBP1A, UTP20, PRMT3, FARSA, MAP3K6, LAS1L, PUS1, HSPE1, SLC29A2, DCTPP1, SUPV3L1, SORD, IMP4, GRWD1, UNG, MPHOSPH10, and NME2) in a cancer sample (such as a prostate cancer sample) can be evaluated using these methods. Immunohistochemical techniques can also be utilized protein detection and quantification. General guidance regarding such techniques can be found in Bancroft and Stevens (Theory and Practice of Histological Techniques, Churchill Livingstone, 1982) and Ausubel et al. (Current Protocols in Molecular Biology, John Wiley & Sons, New York, 1998).

To quantify proteins, a biological sample of a subject that includes cellular proteins can be used. Quantification of enzalutamide resistance-related proteins from one or more enzalutamide resistance-related molecular pathways and/or transcriptional regulatory programs (such as one or more of MYC, SLC19A1, MRTO4, TMEM97, RRP9, PES1, TFB2M, EXOSC5, IPO4, NDUFAF4, NOC4L, SRM, PA2G4, GNL3, NOLC1, WDR43, RABEPK, NOP16, TBRG4, DDX18, NIP7, WDR74, BYSL, HSPD1, PLK4, NOP2, PPAN, NOP56, RCL1, NPM1, AIMP2, RRP12, PPRC1, TCOF1, MCM5, HK2, CBX3, PLK1, PHB, MCM4, CDK4, DUSP2, MYBBP1A, UTP20, PRMT3, FARSA, MAP3K6, LAS1L, PUS1, HSPE1, SLC29A2, DCTPP1, SUPV3L1, SORD, IMP4, GRWD1, UNG, MPHOSPH10, and NME2) can be achieved by immunoassay methods. The amount of enzalutamide resistance-related proteins from one or more enzalutamide resistance-related molecular pathways and/or transcriptional regulatory programs (such as one or more of MYC, SLC19A1, MRTO4, TMEM97, RRP9, PES1, TFB2M, EXOSC5, IPO4, NDUFAF4, NOC4L, SRM, PA2G4, GNL3, NOLC1, WDR43, RABEPK, NOP16, TBRG4, DDX18, NIP7, WDR74, BYSL, HSPD1, PLK4, NOP2, PPAN, NOP56, RCL1, NPM1, AIMP2, RRP12, PPRC1, TCOF1, MCM5, HK2, CBX3, PLK1, PHB, MCM4, CDK4, DUSP2, MYBBP1A, UTP20, PRMT3, FARSA, MAP3K6, LAS1L, PUS1, HSPE1, SLC29A2, DCTPP1, SUPV3L1, SORD, IMP4, GRWD1, UNG, MPHOSPH10, and NME2) can be assessed in a prostate cancer sample from a subject and optionally in prostate cancer samples) from patients known to respond to enzalutamide therapy (or to not respond). The amounts of enzalutamide resistance-related proteins from one or more enzalutamide resistance-related molecular pathways and/or transcriptional regulatory programs (such as one or more of MYC, SLC19A1, MRTO4, TMEM97, RRP9, PES1, TFB2M, EXOSC5, IPO4, NDUFAF4, NOC4L, SRM, PA2G4, GNL3, NOLC1, WDR43, RABEPK, NOP16, TBRG4, DDX18, NIP7, WDR74, BYSL, HSPD1, PLK4, NOP2, PPAN, NOP56, RCL1, NPM1, AIMP2, RRP12, PPRC1, TCOF1, MCM5, HK2, CBX3, PLK1, PHB, MCM4, CDK4, DUSP2, MYBBP1A, UTP20, PRMT3, FARSA, MAP3K6, LAS1L, PUS1, HSPE1, SLC29A2, DCTPP1, SUPV3L1, SORD, IMP4, GRWD1, UNG, MPHOSPH10, and NME2) in the prostate cancer sample from the subject can be compared to levels of the protein(s) found in prostate cancer samples from patients known to respond to enzalutamide therapy (or not respond) or other control (such as a standard value or reference value). A significant increase or decrease in the amount can be evaluated using statistical methods.

Quantitative spectroscopic approaches, such as SELDI, can be also used to analyze expression of enzalutamide resistance-related proteins from one or more enzalutamide resistance-related molecular pathways and/or transcriptional regulatory programs (such as one or more of MYC, SLC19A1, MRTO4, TMEM97, RRP9, PES1, TFB2M, EXOSC5, IPO4, NDUFAF4, NOC4L, SRM, PA2G4, GNL3, NOLC1, WDR43, RABEPK, NOP16, TBRG4, DDX18, NIP7, WDR74, BYSL, HSPD1, PLK4, NOP2, PPAN, NOP56, RCL1, NPM1, AIMP2, RRP12, PPRC1, TCOF1, MCM5, HK2, CBX3, PLK1, PHB, MCM4, CDK4, DUSP2, MYBBP1A, UTP20, PRMT3, FARSA, MAP3K6, LAS1L, PUS1, HSPE1, SLC29A2, DCTPP1, SUPV3L1, SORD, IMP4, GRWD1, UNG, MPHOSPH10, and NME2) in a sample (such as a prostate cancer sample). In one example, surface-enhanced laser desorption-ionization time-of-flight (SELDI-TOF) mass spectrometry is used to detect protein expression, for example by using the ProteinChip™ (Ciphergen Biosystems, Palo Alto, CA). Such methods are well known in the art (for example see U.S. Pat. Nos. 5,719,060; 6,897,072; and 6,881,586). SELDI is a solid phase method for desorption in which the analyte is presented to the energy stream on a surface that enhances analyte capture or desorption.

### V. Samples and Controls

The methods provided herein include detecting expression of enzalutamide resistance-related molecules from one or more enzalutamide resistance-related molecular pathways and/or transcriptional regulatory programs (such as one or more of MYC, SLC19A1, MRTO4, TMEM97, RRP9, PES1, TFB2M, EXOSC5, IPO4, NDUFAF4, NOC4L, SRM, PA2G4, GNL3, NOLC1, WDR43, RABEPK, NOP16, TBRG4, DDX18, NIP7, WDR74, BYSL, HSPD1, PLK4, NOP2, PPAN, NOP56, RCL1, NPM1, AIMP2, RRP12, PPRC1, TCOF1, MCM5, HK2, CBX3, PLK1, PHB, MCM4, CDK4, DUSP2, MYBBP1A, UTP20, PRMT3, FARSA, MAP3K6, LAS1L, PUS1, HSPE1, SLC29A2, DCTPP1, SUPV3L1, SORD, IMP4, GRWD1, UNG, MPHOSPH10, and NME2) in prostate cancer samples. In some examples, the sample is a primary prostate cancer sample. In other examples, the sample is a metastatic prostate cancer sample (such as a sample from a metastatic tumor that originated from a prostate cancer).

In some implementations, the samples are obtained from subjects diagnosed with prostate cancer A “sample” refers to part of a tissue that is either the entire tissue, or a diseased or healthy portion of the tissue. As described herein, prostate cancer samples can be compared to a control. In some implementations, the control is a prostate cancer sample obtained from a subject or group of subjects known to have favorably responded to enzalutamide therapy (or not to have responded to enzalutamide therapy).

In other implementations, the control is a standard or reference value based on an average of historical values. In some examples, the reference values are an average expression value for each of a molecule from enzalutamide resistance-related molecules from one or more enzalutamide resistance-related molecular pathways and/or transcriptional regulatory programs (such as one or more of MYC, SLC19A1, MRTO4, TMEM97, RRP9, PES1, TFB2M, EXOSC5, IPO4, NDUFAF4, NOC4L, SRM, PA2G4, GNL3, NOLC1, WDR43, RABEPK, NOP16, TBRG4, DDX18, NIP7, WDR74, BYSL, HSPD1, PLK4, NOP2, PPAN, NOP56, RCL1, NPM1, AIMP2, RRP12, PPRC1, TCOF1, MCM5, HK2, CBX3, PLK1, PHB, MCM4, CDK4, DUSP2, MYBBP1A, UTP20, PRMT3, FARSA, MAP3K6, LAS1L, PUS1, HSPE1, SLC29A2, DCTPP1, SUPV3L1, SORD, IMP4, GRWD1, UNG, MPHOSPH10, and NME2) in a sample (such as a prostate cancer sample) obtained from a subject or group of subjects known to have favorably responded to enzalutamide therapy (or not to have responded).

Tissue samples can be obtained from a subject, for example, from cancer prostate patients who have undergone tumor biopsy or tumor resection. In some implementations, prostate cancer samples are obtained by biopsy. Biopsy samples can be fresh, frozen or fixed, such as formalin-fixed and paraffin embedded. Samples can be removed from a patient surgically, by extraction (for example by hypodermic or other types of needles), by microdissection, by laser capture, or by other means.

In some examples, proteins and/or nucleic acid molecules (e.g., DNA, RNA, mRNA, and cDNA) are isolated or purified from the prostate cancer sample. In some examples, the sample (such as a prostate cancer sample) is used directly, or is concentrated, filtered, or diluted.

### VI. Methods of Treating Enzalutamide-Resistant Prostate Cancer

As described herein, inhibition of Myc molecular pathway or NME2 transcriptional regulatory program can restore sensitivity or responsiveness to prostate cancer that was enzalutamide resistant. Therefore, provided are methods of treating a subject with enzalutamide-resistant prostate cancer, for example, a subject that has received enzalutamide therapy and undergone disease progression. In particular examples, the subject with enzalutamide resistant prostate cancer has a prostate tumor or metastasis that has increased expression of Myc and NME2 compared to a control (such as a sample from a subject who positively responds to enzalutamide therapy). The methods include administering to the subject an inhibitor of a Myc molecular pathway or a NME2 transcriptional regulatory program. In some implementations, the inhibitor of the Myc molecular pathway inhibits Myc expression or activity. In one example, the inhibitor of the Myc molecular pathway is Myc-i975. Other Myc inhibitors can also be used, such as IIA6B17, NY2267, 10058-F4, 10074-G5, 3jc48-3, JY-3-094, 3JC-91-2, Mycro1, Mycro2, Mycro3, MYCMI-6, MYCi361, KJ-Pyr-9, celastrol, JKY-2-169, EN4, Omomyc, monoclonal antibodies, and others. In other implementations, the inhibitor of the NME2 transcriptional regulatory program inhibits NME2 expression or activity (such as an antisense oligonucleotide or siRNA that specifically binds to an NME2 nucleic acid). In one example, the inhibitor of the NME2 transcriptional regulatory pathway is an siRNA, such as SEQ ID NO: 7 or SEQ ID NO: 8. In other implementations, the inhibitor of the NME2 transcriptional regulatory pathway is an shRNA, such as SEQ ID NO: 9. In some implementations, the methods further include treating the subject with enzalutamide.

### EXAMPLES

The following examples are provided to illustrate certain particular features and/or implementations. These examples should not be construed to limit the disclosure to the particular features or implementations described.

### Example 1

### Materials and Methods

### I. Computational Methods

Datasets utilized: Datasets utilized for network construction, mining, validation, and negative control analysis are summarized in Table 1.

(i) Dataset to associate activity levels of MYC pathway with response to Enzalutamide: To determine if increased activity levels of MYC pathway were associated with Enzalutamide resistance, we utilized Enzalutamide-associated CRPC metastatic samples from Abida et al. (PNAS 116:11328, 2019) cohort (fresh-frozen needle biopsies), profiled on Illumina HiSeq 2500 and downloaded from github.com/cBioPortal/datahub/tree/master/public/prad_su2c_2019. We specifically selected samples that at the time of biopsy (sample collection) were ARSI-naïve (not subjected to any ARSI treatment), treated with Enzalutamide after sample collection, and then followed up for Enzalutamide-associated disease progression (n=22, one sample per patient, as described in Abida et al.). In this sub-group, the mean age at diagnosis was 59 years with a standard deviation of 6.85, the mean age at biopsy was 67.6 years with a standard deviation of 8.3, and the mean prostate-specific antigen (PSA) was 189.4 ng/ml with a standard deviation of 526.18. Metastatic composition of this sub-group included lymph node (n=13), bone (n=6), lung (n=1), other soft tissue (n=1), and liver (n=1) samples. We utilized Enzalutamide-associated disease progression, defined as the time on Enzalutamide treatment without being subjected to another agent such as taxane, as the clinical end-point (as defined and suggested in Abida et al.).

(ii) Dataset to associate activity levels of MYC pathway with response to Abiraterone: To determine if elevated activity levels of MYC pathway were specifically associated with Enzalutamide (and not Abiraterone) resistance, we utilized Abiraterone-associated metastatic CRPC sample from Abida et al. cohort. We specifically selected samples that at the time of biopsy (sample collection) were ARSI-naïve (as above), treated with Abiraterone after sample collection, and then followed up for Abiraterone-associated disease progression (n=33, one sample per patient) for negative control analysis. The mean age at diagnosis for this patient sub-group was 61.38 years with a standard deviation of 5.94, the mean age at biopsy was 66.73 years with a standard deviation of 7.02, and the mean PSA was 51.4 ng/ml with a standard deviation of 91.05. Metastatic composition of this sub-group included lymph node (n=18), bone (n=11), liver (n=2), and other soft tissue (n=2) samples. We utilized Abiraterone-associated disease progression, defined as the time on Abiraterone treatment, without being subjected to other agents such as taxane, as the clinical end-point (as defined and suggested in Abida et al.).

(iii) Dataset for network reconstruction: To construct a mechanism-centric network, we utilized the Stand Up to Cancer (SU2C) East Coast cohort (Robinson et al., Cell 161:1215-1228, 2015; Abida et al.), profiled on Illumina HiSeq 2500 and downloaded from dbGaP phs000915.v2.p2. This cohort included metastatic CRPC samples, obtained as fresh-frozen needle biopsies. We examined 280 samples available at dbGaP, and to avoid any overlap with treatment-associated analysis in Abida et al. (which we have utilized in part for validation and in part for a negative control), we removed all SU2C East Coast cohort samples that were present in Abida et al. (n=29). Subsequently, we also removed samples that were duplicated (when the same sample was sequenced by different facilities) and selected one sample per patient to avoid signal duplication for our final network-building. Our final cohort comprised of 153 patients with a mean age at diagnosis of 59.2 years with a standard deviation of 8.38, a mean age at biopsy of 66.1 years with a standard deviation of 8.07, and a mean PSA of 234.5 ng/ml with a standard deviation of 1574.4. Metastatic composition of this cohort included adrenal (n=1), bone (n=39), liver (n=26), lymph node (n=57), other soft tissue (n=19), prostate (n=4), lung (n=2) and unknown origin (n=5) samples. At the time of biopsy (sample collection), patients either were exposed to ARSI (n=67), were ARSI-naïve (n=75), were on treatment (n=4), or their treatment was unknown (n=7).

(iv) Datasets for network mining: For network mining (query/interrogation), we utilized LNCaP cell line samples from Kregel et al. (Oncotarget 7:26259-26274, 2016) (n=12), that were profiled with the HumanHT-12 v4 Expression BeadChip and downloaded from GEO GSE78201. These dataset included three phenotypes: (i) LNCaP cells subjected to DMSO (referred to as Intact to indicate that they were not subjected to Enzalutamide treatment) (n=4); (ii) LNCaP cells subjected to Enzalutamide for 48 hours and sensitive to it (referred to as Enzalutamide-sensitive, EnzaSens) (n=4); and (iii) LNCaP cells subjected to Enzalutamide for 6 months and having developed resistance to it (referred as Enzalutamide-resistant, EnzaRes) (n=4).

(v) Datasets for clinical validation: For validation purposes, we utilized (i) He et al. (Nature Medicine 27:426-433, 2021) (ii) Enzalutamide-associated Abida et al., and (iii) SU2C West Coast (Quigley et al., Cell 174:758-769.e759, 2018; Aggarwal et al., Eur. Urol. Focus 2:469-471, 2016) datasets. First, to confirm that upregulation of the NME2 transcriptional regulatory program and MYC pathway characterize Enzalutamide-naïve samples (before patients were exposed to Enzalutamide) from patients that were later exposed to Enzalutamide and eventually failed it, we selected two sequential single-cell samples from the same CRPC patient (01115655) from He et al. cohort. These samples were profiled on Illumina NextSeq 500 and downloaded from singlecell.broadinstitute.org/single_cell/study/SCP1244/transcriptional-mediators-of-treatment-resistance-in-lethal-prostate-cancer. In particular, the first sample was collected before the patient was subjected to Enzalutamide and second sample was collected after the same patient received Enzalutamide and developed resistance to it. Both samples were collected from the lymph-node metastatic site.

Findings from He et al. were confirmed in Enzalutamide-associated Abida et al. cohort. Briefly, we selected a subset of patients that were ARSI-naïve at biopsy, treated with Enzalutamide after sample collection, and subsequently monitored for Enzalutamide-associated disease progression (n=22, as described above).

Further, we validated the predictive ability of NME2 TR and MYC pathway in SU2C West Coast cohort, which comprises of samples from CRPC patients (obtained from fresh frozen image guided core needle biopsies), profiled on Illumina HiSeq 2500 or NextSeq 500 and downloaded from GDC (portal.gdc.cancer.gov/projects/WCDT-MCRPC). The samples in this cohort were subjected to Enzalutamide and/or Abiraterone either before biopsy (sample collection) or after biopsy (sample collection). Subsequently, all patients were monitored for disease progression (n=83, one sample per patient). The mean age for the patients in this cohort was 70.59 years with standard deviation of 8.14. Alongside the patients in this cohort were from various races, including, white (n=70), Asian (n=2), African American (n=5) and unknown (n=6). Metastatic composition of this cohort included bone (n=36), liver (n=7), lymph node (n=31), and unknown (n=9). Further, samples were obtained from patients who were either in M1b stage (i.e., when prostate cancer has spread to bone, n=36) or M1c stage (when prostate cancer has spread to other parts of the body, n=47). We utilized treatment-associated disease progression (defined as an increase in PSA level (minimum 2 ng/mL) that has risen at least twice in an interval of least one week or soft tissue progression (nodal and visceral) based on RECIST v1.1) as the clinical end-point (as defined and suggested in Quigley et al. and Aggarwal et al.).

(vi) Datasets for negative control analysis: To evaluate if the predictive ability of NME2 TR and MYC pathway are indeed Enzalutamide specific, we utilized (i) Abiraterone-associated Abida et al. cohort (as described above); and (ii) PROMOTE (Wang et al., Ann. Oncol. 29:352-360, 2018) cohort, as negative controls. As described above, Abiraterone-associated Abida et al. cohort included ARSI-naïve CRPC samples obtained at biopsy, treated with Abiraterone after sample collection, and subsequently monitored for Abiraterone-associated disease progression (n=33, as described above).

PROMOTE cohort included samples from patients with CRPC profiled on Illumina HiSeq 2500 and downloaded from dbGaP phs001141.v1.p1. These samples were obtained at biopsy from different metastatic sites (n=77, one sample per patient), including bone (n=56), soft tissue (n=2), liver (n=2), prostate bed (n=2), lymph-node (n=14), and lung (n=1) and were ARSI-naïve at the time of sample collection. After sample collection, the patients were subjected to Abiraterone for 12 weeks, and were assessed for Abiraterone-associated disease progression right after that, which was defined based on the score that combined serum PSA level, bone and CT imaging, and symptom assessment at week 12. Patients that developed disease progression at week 12 were classified as non-responders (n=32) and those that did not develop disease progression at week 12 were classified as responders (n=45).

Data download, processing, and normalization: Abida et al. RNA-seq samples profiled on Illumina HiSeq 2500, were downloaded from github.com/cBioPortal/datahub/tree/master/public/prad_su2c_2019 as Fragments Per Kilobase of transcript per Million mapped reads (FPKM). The clinical and treatment data were downloaded from the supplementary material of Abida et al. and from cBioPortal (cbioportal.org/).

SU2C East Coast cohort RNA-seq samples profiled on Illumina HiSeq 2500, were requested and downloaded from dbGaP phs000915.v2.p2 as SRA files using the prefetch command and were converted to FASTQ files utilizing the fastq-dump command from sra toolkit (version 10.8.2). Following this, the FASTQ files were aligned to a reference genome hg19 using STAR aligner with the quantMode option, which generated raw count files. The raw counts were normalized using R DESeq package for further statistical analysis. The clinical data were obtained from the supplementary material of Abida et al. and from cBioPortal.

Kregel et al. LNCaP cell line samples were profiled on HumanHT-12 v4 Expression BeadChip Kit and their quantile-normalized gene expression data were downloaded from GEO GSE78201. The phenotype information was obtained from GEO GSE78201.

He et al. single-cell RNA-seq samples profiled on Illumina NextSeq 500, were downloaded from singlecell.broadinstitute.org/single_cell/study/SCP1244/transcriptional-mediators-of-treatment-resistance-in-lethal-prostate-cancer, as single-cell Transcripts Per Million (TPM) data matrix. The clinical data were obtained from the main body and supplementary material of He et al.

SU2C West Coast cohort RNA-seq samples profiled on either Illumina HiSeq 2500 or NextSeq 500 were downloaded from GDC (portal.gdc.cancer.gov/projects/WCDT-MCRPC) as BAM files. These BAM files were then converted to FASTQ files utilizing bam2fastq from bedtools. Subsequently, the FASTQ files were aligned to a reference genome hg19 using STAR aligner with the quantMode option, which generated raw count files. The raw counts were normalized using R DESeq for further statistical analysis. The clinical and treatment data were obtained from GDC (portal.gdc.cancer.gov/projects/WCDT-MCRPC).

PROMOTE RNA-seq samples profiled on Illumina HiSeq 2500, were requested and downloaded from dbGaP phs001141.v1.p1 as SRA files using the prefetch command and then converted to FASTQ files using the fastq-dump command from sra toolkit (version 10.8.2). Subsequently, the FASTQ files were aligned to the reference genome hg19 using STAR aligner with the quantMode option to generate raw count files. The raw count files were normalized using R DESeq package. The clinical data were obtained from dbGaP phs001141.v1.p1.

Estimating activity levels of molecular pathways: A list of molecular pathways and their corresponding genes were obtained from Molecular Signatures Database (MSigDB), available from Broad Institute, and included C2 pathway collection (KEGG, BioCarta, and Reactome) and Hallmark (Liberzon et al., Cell Systems 1:417-425, 2015) gene sets. To estimate activity levels of each molecular pathway, we utilized signature-based or single-patient (single-sample) based Gene Set Enrichment Analysis (GSEA), similarly to Epsi et al. (Communications Biology 2:334, 2019) and Rahem et al. (EBioMedicine 61:103047, 2020). For the signature-based GSEA analysis, a signature of interest (e.g., defined as a list of genes ranked by their differential expression using two-tailed Welch t-test between any two phenotypes of interest, such as Enzalutamide-resistant and Enzalutamide-sensitive phenotypes) is used as a reference signature and genes from a specific pathway are used as a query gene set. For single-patient (single-sample) GSEA analysis, gene expression profiles were scaled/standardized (i.e., z-scored) on gene-level so that mean of values for each gene was 0 and the standard deviation was 1, allowing for comparison of gene ranks across different samples. A single-sample signature was defined as a list of genes ranked by their z-scores and utilized as a reference signature in single-sample GSEA analysis (pathway genes were utilized for query, in the same manner as above). For signature-based and single-sample GSEA analysis, Normalized Enrichment Score (NES) and p-values were estimated using 1,000 gene permutations. NESs from this analysis were utilized as pathway activity values, where positive NES corresponds to an enrichment of pathway genes in the over-expressed part of the signature and negative NES corresponds to an enrichment of pathways genes in the under-expressed part of the signature.

Estimating activity levels of Transcriptional Regulatory programs: To estimate the activity levels of transcriptional regulators we utilized MARINa (for a signature-based analysis) and VIPER (for a single-sample-based analysis). Signatures were defined in the same manner as for the pathway enrichment analysis and were utilized as a reference for MARINa/VIPER. Instead of utilizing pathway data, MARINa and VIPER analyses require tissue-specific prostate cancer transcriptional regulatory network (interactome), as reconstructed previously in Aytes et al. (Cancer Cell 25:638-651, 2014). This interactome comprises of transcriptional regulators (TR, transcription factors and co-factors) and their potential transcriptional targets, connected by the transcriptional regulatory relationships. During MARINa/VIPER analysis, these transcriptional targets (for each transcriptional regulator separately) are utilized as a query gene set. We refer to the TR and the set of its corresponding transcriptional targets as a transcriptional regulatory program. Similar to GSEA, NESs/z-scores from MARINa and VIPER analysis were utilized to define activity levels of TRs. MARINa was implemented using msviper function and VIPER was implemented using viper function from R VIPER package in Bioconductor.

TR-2-PATH: reconstruction of a mechanism-centric regulatory network: To identify potential regulatory relationships between molecular pathways and their upstream transcriptional regulatory programs in CRPC patients, we have reconstructed a CRPC-specific mechanism-centric regulatory network, using newly developed TR-2-PATH method. In this network, each node represents a mechanism: a molecular pathway or transcriptional regulatory program. SU2C East Coast cohort (as described above) was first scaled/standardized on the gene level and then subjected to single-sample pathway enrichment analysis (as described above) and single-sample transcriptional regulatory analysis (as described above). We then defined activity vectors for each molecular pathway (where each pathway vector corresponds to the NESS for this pathway across all patients in the SU2C East Coast cohort) and for each TR program (where each TR vector corresponds to the NESs/z-scores for this TR across all patients in SU2C East Coast cohort). Specifically, let us assume that we have n samples. If the activity level of pathway i in sample j is NES_{i,j}, then the activity vector for pathway i, P_{i activity} is defined as.

$${P}_{i activity} = [ \begin{matrix}{NES}_{i , 1} \\ {NES}_{i , 2} \\ . \\ . \\ . \\ {NES}_{i , n}\end{matrix} ]$$

Similarly, if the activity level of a TR t in a sample j is a_{t,j}, then the activity vector for TR t, TR_{t activity} is defined as

$${TR}_{t activity} = [ \begin{matrix}\text{?} \\ \text{?} \\ . \\ . \\ . \\ \text{?}\end{matrix} ]$$

$$\text{?} \text{indicates text missing or illegible when filed}$$

To estimate potential regulatory relationships between transcriptional regulatory programs and molecular pathways, we first performed a pairwise comparison of each TR activity vector and each pathway activity vector using linear regression analysis, where a TR activity vector was used as a predictor variable (independent variable) and pathway activity vector was used as a response variable (dependent variable), as below. For each pathway i and TR t:

$${P}_{i activity} = α + β * T \text{?}$$

$$\text{?} \text{indicates text missing or illegible when filed}$$

The positive Beta (β) coefficient from the linear regression analysis (which corresponds to a positive slope for the fitted line between TR activity vector and pathway activity vector) indicated a positive relationship/association from the TR to the pathway and a negative Beta coefficient (negative slope) indicated a negative relationship/association from the TR to the pathway. Following the regression analysis for all TR-pathway pairs, we subjected it to multiple hypotheses FDR correction, which was performed for each pathway separately. If this relationship showed FDR <0.05, it was added as an edge to the final network. Otherwise, it was discarded. Linear regression analysis was performed using the R lm function and multiple hypotheses testing per pathway was performed using the R p.adjust function.

Bootstrap analysis for the mechanism-centric regulatory network: To evaluate if the edges in the mechanism-centric regulatory network could be “recovered” in the presence of noise (re-sampling), we performed bootstrap analysis. For this, SU2C East Coast cohort gene expression profiles (n=153) were sampled with replacements 100 times. Each sampled/bootstrapped gene expression profile was then used to reconstruct a bootstrapped mechanism-centric regulatory network using the TR-2-PATH method (as above). We then utilized results from these 100 networks to assign weights to each edge, which was defined as the number of times this edge appears (was recovered) across 100 bootstrapped networks (edge frequency). In particular, the edge weights were defined as the percent (%) of times an edge identified in the original network was also identified across the bootstrapped networks while maintaining the same direction of the relationship (positive/negative) between a particular TR program and a particular molecular pathway, across all 100 bootstrapped networks. These edge weights were then added to the original network (making it a weighted mechanism-centric network) and further utilized in the network query step.

The R functions hist and density were utilized to depict weight distributions. To cluster the molecular pathways based on their edge weights, we utilized t-distributed stochastic neighbor embedding clustering (t-SNE), a common dimensionality reduction technique that clustered pathways with similar edge weight patterns as nearby points and pathways with dissimilar edge weight patterns as distal points. t-SNE was implemented using the Rtsne function from R Rtsne package.

Network mining I: Identifying differentially altered sub-networks: To identify parts of the mechanisms-centric network (sub-networks comprising of the molecular pathways and their upstream TR programs) that significantly alter their activity across the response to Enzalutamide, we queried (mined) the mechanism-centric regulatory network using signatures of Enzalutamide-response. In particular, we specifically utilized gene expression profiles from Kregel et al. (as described above), which consists of (i) Intact (DMSO subjected) LNCaP cells (n=4), (ii) Enzalutamide-sensitive (EnzaSens) LNCaP cells (n=4); and (iii) Enzalutamide-resistant (EnzaRes) LNCaP cells (n=4). We hypothesized that if a particular sub-network is up-regulated (positive NES) in the intact state, then becomes down-regulated (negative NES) in the sensitive state, yet “recovers” and again become up-regulated (positive NES) in the resistant state (we call this “up-down-up” behavior), then such sub-network is important in Enzalutamide-resistance and could potentially constitute a functional marker and a therapeutic vulnerability. To identify such sub-networks and establish the significance of this change, we defined two gene expression query signatures (i) signature between intact and sensitive phenotype; and (ii) signature between sensitive and resistant phenotype. These signatures were defined utilizing two-tailed Welch t-test and implemented using the R t.test function.

To identify sub-networks with such “up-down-up” behavior, we evaluated their enrichment in the “intact to sensitive” signature (looking for “up-down” behavior, corresponding to the down-regulation as a result of response to Enzalutamide) and enrichment in the “sensitive to resistant” signature (looking for “down-up” behavior, corresponding to the subsequent up-regulation as a result of resistance to Enzalutamide). To achieve this, we first estimated pathway activity levels and TR activity levels in each signature and overlayed them with our mechanism-centric regulatory network relationships/structure to identify parts of the network that exercise “up-down-up” behavior, as described above. To estimate if such “up-down-up” changes were statistically significant, we performed pathway-on-pathway and TR-on-TR GSEA, where pathways from “intact to sensitive” signature were compared to pathways from “sensitive to resistant” signature (same for the TR programs). Parts of the network with significant negative enrichment in “intact to sensitive” signature and significant positive enrichment in “sensitive to resistant” signature (GSEA p-value <0.001) were utilized for Network mining step II.

### Network Mining II: Prioritization of Upstream Regulatory Programs

Variance Inflation Factor analysis: Sub-networks identified in “Network mining I” include molecular pathways and their potential upstream TR programs. Such TR programs might exercise multi-collinearity in their effect on the pathway and could obstruct further statistical analysis (by making results not interpretable), yet deserve to remain in the analysis (as opposed to simply being eliminated). First, to check for multi-collinearity among TRs, we subjected the activity level of these TRs to Variance Inflation Factor analysis (VIF) in the SU2C East Coast cohort. VIF runs a multivariable regression analysis, iteratively using each TR (activity vector) as a response variable and activity vectors from the rest of the TRs as predictor variables. The percentage of variation that the predictor variables could explain about the response variable is defined by the coefficient of determination, R², where higher R² values indicate a higher degree of multi-collinearity and VIF is defined as 1/(1−R). Typically, the multi-collinearity is observed if VIF >10. VIF analysis was implemented utilizing the vif function from the R usdm package.

PLS regression analysis: To address TR multi-collinearity, we developed a Partial Least Squares (PLS)-inspired method. To prioritize the effect of TR programs on a specific pathway i, our approach considers TR activity vectors TR_{t activity}, t=1 . . . m (where m is the number of TRs upstream of a specific pathway i) as predictor variables and utilizes a pathway i activity vector P_{t activity} as a response variable. TR activity vectors are then regressed (linear regression) on the pathway vector so that their β coefficients (slopes), indicating the effect of each TR on a pathway i, are denoted as weights wₜ. Next, utilizing the TR activity vectors and weights associated with each TR, first latent variable LV1 is defined as:

$$LV 1 = \text{?} T \text{?} * \text{?}$$

$$\text{?} \text{indicates text missing or illegible when filed}$$

Further, the contribution (also referred to as loadings) of each TR on the LV1 is determined through a multivariable regression analysis, where the activity vectors of all the transcriptional regulators TR_{t activity} are utilized as independent variables and the LV1 is utilized as a dependent variable. The B coefficients associated with each TR in this multivariable analysis, indicating the contribution of each TR_{t activity} to LV1, adjusted for the effect of all other TRs, as denoted as loadings. Loadings are most often utilized in social science analyses.

This latent variable LV1 is then “subtracted” from the TR activity vectors and the pathway i activity vector, leaving the residuals to be utilized for defining the next latent variable. In particular, the first latent variable LV1 is utilized as an independent variable to be regressed on the activity vectors of each TR program TR_{t activity} as well as activity of the molecular pathway P_{i activity}, so that the residuals from this analysis explain amount of information that has not been explained by LV1. The residuals are then utilized to define the second latent variable LV2 in the similar fashion. This process is repeated until latent variables can explain a significant amount of information about a pathway i. PLS was implemented utilizing the plsreg1 function from the R plsdepot package.

PLS-inspired circle of correlation analysis: Identified latent variables do not express collinearity or multi-collinearity and are utilized as axes to build a “circle of correlation,” which depicts the association of TR programs and a specific pathway i (defined as arrows on the circle of correlation) to each latent variable. In particular, axes of the circle of correlation depict Pearson correlation r values, defined between latent variables and TR/pathway activity vectors. Each TR and a pathway i are indicated as arrows on the circle of correlation, with x and y coordinates that correspond to the values of Pearson correlation between their vectors and the latent variables.

To identify TRs that affect a specific pathway i as a group, we developed a method that utilized unsupervised hierarchical clustering on the degree of closeness (angle) between TR and pathway arrows so that TRs in high proximity to one another (thus having similar effects on latent variables) are grouped as they express simultaneous effect on the pathway i. In particular, for each TR and pathway arrow we first calculated their angle of inclination (i.e., cos⁻¹ θ). To calculate the cos⁻¹ θ we utilized R acos function. Following this, angle of inclination in radian was converted to a degree using the rad2deg function from the R rCAT package. To determine the degree of closeness, we subtracted the angle of inclination of each TR arrow from angle of inclination of a pathway i arrow. These degrees of closeness for TRs were then subjected to hierarchical clustering, which identified groups of TR programs with similar effects on the pathway i. For hierarchical clustering we utilized the R hclust function.

Prioritizing TR groups: The TR groups/clusters (which also include groups with one TR) are then “prioritized” based on their effect on a pathway i using “effect scores,” which are defined as a combination of (i) degree of closeness between a TR group/cluster and a pathway i on the circle of correlation; (ii) association (Pearson correlation r) between a TR group/cluster and each evaluated latent variable; and (iii) edge weight between a TR group/cluster and a pathway i from the TR-2-PATH mechanism-centric network reconstruction step. For clusters that contained more than one TR, average values for all TRs in that cluster were considered. Each of these categories assigned a “rank” for each cluster and then ranks were combined (using geometric mean) to define the final effect score for each cluster. Geometric mean was implemented utilizing the geometric.mean function from the R psych package.

Validation in independent cohorts and Enzalutamide specificity analysis: For validation and negative control analysis, we utilized He et al., Abida et al., SU2C West Coast and PROMOTE cohorts. Clinical characteristics and data normalization for these cohorts are described above and in Table 1.

In He et al. (single-cell profiles), we reproduced data analysis performed by in the original manuscript. In particular, we applied UMAP dimensionality reduction technique on single-cell Transcripts Per Million (TPM) data for each sample of the selected patient. We then utilized the AR activity and CK8 and CD45 expression on the UMAP projected data to identify adenocarcinoma cell clusters. Next, we estimated NME2 TR activity and MYC pathway activity on a single-cell level, in a manner similar to the single-sample analysis (as described above) and compared their activities between adenocarcinoma cells and the rest of the cells utilizing one-tailed Welch t-test, using the t.test function in R.

In Abida et al., we subjected the cohort samples to a single-sample pathway and single-sample TR analysis to estimate activity levels of MYC pathway and NME2 TR program across all samples. For Enzalutamide-associated subset, we first performed Cook's distance analysis to identify outliers that can influence the regression analysis results (no outliers identified) utilizing R cooks.distance function. Following this, we performed association analyses between activity vectors of NME2 TR and MYC pathway using the R cor.test function. Next to identify patients with high-NME2 TR and high-MYC pathway activities in Enzalutamide-associated subset, we performed hierarchical and kmeans clustering on MYC pathway and NME2 TR activity vectors. For Abiraterone-associated subset, we also performed the Cook's distance analysis (one outlier identified and removed) to identify outliers, followed by identification of patients with high-NME2 TR and high-MYC pathway activities. To identify patients with high-NME2 TR and high-MYC pathway activities, we applied the same thresholds that was estimated in the Enzalutamide-associated subset. Hierarchical clustering was implemented using the R hclust function and kmeans clustering was performed using the R kmeans function and identified two clusters of patients (i) patients with high-NME2 activity and high-MYC pathway activity and (ii) the rest of the patients (e.g., patients with low-NME2 and low-MYC pathway activity; patients with low-NME2 and high-MYC pathway activity; and patients with high-NME2 and low-MYC pathway activity). Further, to evaluate the difference in treatment response between the two identified groups, we utilized Kaplan-Meier survival analysis and Cox proportional hazards model analysis, where treatment-associated disease progression (as described above) was utilized as the clinical end-points, as defined in Abida et al. For Kaplan-Meier survival analysis, we utilized the Surv and the ggsurvplot functions from R survival and survminer packages, respectively. The Cox proportional hazards model analysis was adjusted for age and Gleason score and utilized the coxph function from the R survival package.

In SU2C West Coast cohort, similar to analysis on Abida et al, we subjected the cohort samples to a single-sample pathway and single-sample TR analysis to estimate activity levels of MYC pathway and NME2 TR program across all samples. As above, we first performed Cook's distance analysis to identify outliers (three outliers identified and removed) using R cooks.distance function followed by performing association analyses between activity vectors of NME2 TR and MYC pathway using the R cor.test function. Next, to identify patients with high-NME2 TR and high-MYC pathway activities we utilized hierarchical and kmeans clustering on MYC pathway and NME2 TR activity vectors. Hierarchical clustering was implemented using R hclust function and kmeans clustering was implemented using the R kmeans function and identified two clusters of patients (i) patients with high-NME2 activity and high-MYC pathway activity and (ii) the rest of the patients (e.g., patients with low-NME2 and low-MYC pathway activity; patients with low-NME2 and high-MYC pathway activity; and patients with high-NME2 and low-MYC pathway activity). Further, to evaluate the difference in treatment response between the two identified groups, we utilized Kaplan-Meier survival analyses36 and Cox proportional hazards model analysis, where treatment-associated disease progression (as described earlier) was utilized as the clinical end-points. For Kaplan-Meier survival analysis, we utilized the Surv and the ggsurvplot functions from the R survival and survminer packages respectively. The Cox proportional hazards model analysis was adjusted for race, Mstage, age and metastatic site and utilized the coxph function from the R survival package.

In PROMOTE cohort, we first performed Cook's distance analysis to identify outliers as above (two outliers identified and removed) using R cooks.distance function. Since PROMOTE cohort has binary outcomes (responders vs non-responders), to evaluate the ability of NME2 TR and MYC pathway activities to classify patients based on their binary response to Abiraterone treatment, we performed ROC analysis using a multiplicative logistic regression model, where the product of activity level of the NME2 TR program and activity level of the MYC pathway was utilized as predictor (independent) variable and responder/non-responder classification was utilized as response (dependent) variable. ROC curves were evaluated using area under the curve (AUROC), with AUROC=0.5 being a random classifier. The logistic regression analysis was implemented using the R glm function and ROC analysis was implemented using the roc function from the R PROC package.

Comparison to markers of aggressiveness and therapeutic response: To compare the ability of MYC and NME2 to predict Enzalutamide resistance to the predictive ability of known transcriptomic and genomic markers of aggressiveness and therapeutic response we utilized patients from Enzalutamide-associated Abida et al. cohort (as described above). In particular, comparisons were done in two ways: (i) comparison between high-NME2 and high-MYC pathway patients and the rest of the patients (“others”), as described above using two-tailed Welch t-test (for transcriptomic markers) and Fisher exact test151 (for genomic markers); and (ii) direct independent association with the Enzalutamide-associated disease progression using Cox proportional hazards model. For transcriptomic markers, we utilized their gene expression/normalized counts. For genomic markers, we utilized genomic alterations (obtained from cbioportal), including deep and shallow deletions, gains, and amplifications, as available in cbioportal. Two-tailed Welch t-test was implemented using the R t.test function, Fisher exact test was implemented using the R fisher.test function, and Cox proportional hazards model analysis was implemented using the coxph function from the R survival package.

Comparative analysis to gene-centric computational methods: To evaluate if TR-2-PATH mechanism-centric predictions (activity levels of NME2 TR and MYC pathway) outperform predictive ability of commonly used gene-centric methods, we compared TR-2-PATH to differential expression analyses, Random (survival) Forests (RF), and Support Vector Machine (SVM) methods all utilized on the Enzalutamide-associated Abida et al. cohort. For differential gene expression analysis, we considered genes that were differentially expressed between the three phenotypes (Intact, EnzaSens, and EnzaRes) in the mining step I and considered genes at (i) Welch t-test p-value <0.05; (ii) top 470 differentially expressed genes (comparable to the total number of target genes and pathway genes used for activity estimation) and not excluding target/pathway genes from NME2 TR and MYC pathway; (iii) top 470 differentially expressed genes, excluding target/pathway genes from NME2 TR and MYC pathway. For RF and SVM analysis, we utilized 470 genes from (iii) to avoid overfitting and then selected top 10 most significant genes/features from the outputs. Final gene list from each of these analyses were utilized to cluster patients using hierarchical and kmeans clustering (as above), and then subjected these groups to Kaplan-Meier survival analysis and Cox proportional hazards model analysis. For Kaplan-Meier survival analysis, we utilized the Surv and the ggsurvplot functions from the R survival and the survminer packages, respectively. Additionally, for Cox proportional hazards model analysis, we utilized the coxph function from the R survival package. For adjusted Cox proportional hazards model analysis, the model was adjusted for age and Gleason score. Random (survival) Forests were constructed utilizing rfsrc function from R randomForestSRC package. The tuning parameters for Random (survival) Forests included (i) the maximum number of trees (“ntrees”), (ii) the number of variables assessed at each split (“mtry”), and (iii) maximum number of samples in the terminal (leaf) nodes (“nodesize”). The optimization of mtry and nodesize variables was performed utilizing tune function from R randomForestSRC package, which determined optimal value for mtry as 100 and nodesize as 5 and iterations of ntrees converged to a stable C-index around 3000, thus 3000 was selected as an optimal value for ntrees. For SVM, we utilized fit function from R rminer package with default parameters.

Data visualization: We utilized the geom_violin and the geom_boxplot function from the ggplot2 in R for data visualization.

TABLE 1
Description of datasets

| | Description and use in this | | | | | Reference |
|---|---|---|---|---|---|---|
| Dataset | study | n | Type of data | Platform | Geo/Link | (PubMed Id) |
| SU2C East | Description: | 153 | RNA | Illumina | dbGaP | PMID: 26000489 |
| Coast | Samples of CRPC patients | | sequencing | HiSeq 2500 | phs000915.v2.p2 | PMID: 31061129 |
| cohort | obtained from biopsies of | | | | | |
| | metastatic tissues. | | | | | |
| | Metastatic sites of biopsy | | | | | |
| | includes, adrenal, bone, liver, | | | | | |
| | lymph node, other soft | | | | | |
| | tissue, prostate, lung and | | | | | |
| | unknown | | | | | |
| | Patients in this cohort are | | | | | |
| | either ARSI exposed or ARSI | | | | | |
| | naive. | | | | | |
| | Use: | | | | | |
| | Network reconstruction | | | | | |
| Kregel et | Description: | 12 | Microarray | HumanHT- | GEO | PMID: 27036029 |
| al | Samples of LNCaP cell line | | gene | 12 v4 | GSE78201 | |
| | from untreated phenotype, | | expression | Expression | | |
| | samples of LNCaP cell line | | | BeadChip Kit | | |
| | from sensitive phenotype | | | | | |
| | and samples of LNCaP cell | | | | | |
| | line from resistant | | | | | |
| | phenotype. | | | | | |
| | Use: | | | | | |
| | Network mining | | | | | |
| He et al | Description: | 2 | Single cell | Illumina | https://singlecell.broadinstitute.org/ | PMID: 33664492 |
| | Sequential samples from a | | Transcripts | NextSeq 500 | single_cell/ | |
| | patient (01115655) that | | Per Millions | | study/SCP1244/ | |
| | failed enzalutamide | | (TPM) | | transcriptional- | |
| | One sample was collected | | | | mediators- | |
| | before the patient was | | | | of- | |
| | subjected to enzalutamide | | | | treatment- | |
| | One sample collected after | | | | resistance- | |
| | the patient developed | | | | in-lethal- | |
| | resistance to Enzalutamide | | | | prostate- | |
| | Use: | | | | cancer | |
| | Clinical Validation | | | | | |
| Abida et al | Description: | 22 | Fragments | Illumina | https://github.com/ | PMID: 31061129 |
| | Samples of patients with | | Per Kilobase | HiSeq 2500 | cBioPortal/ | |
| | CRPC that were ARSI-naive at | | of transcript | | datahub/ | |
| | biopsy | | per Million | | tree/master/ | |
| | After sample collection, | | mapped | | public/ | |
| | patients were treated with | | reads (FPKM) | | prad_su2c_2019 | |
| | Enzalutamide | | | | | |
| | Use: | | | | | |
| | Clinical Validation | | | | | |
| SU2C | Description: | 83 | RNA | Illumina | https://portal.gdc.cancer.gov/ | PMID: 3003370 |
| West | Samples of patients with | | sequencing | HiSeq 2500 | projects/WCDT- | PMID: 28723508 |
| Coast | CRPC that were either | | | NextSeq 500 | MCRPC | |
| cohort | treated with ARSI before | | | | | |
| | sample collection of were | | | | | |
| | treated with ARSI after | | | | | |
| | sample collection | | | | | |
| | Use: | | | | | |
| | Clinical Validation | | | | | |
| Abida et al | Description: | 33 | Fragments | Illumina | https://github.com/ | PMID: 31061129 |
| | Samples of patients with | | Per Kilobase | HiSeq 2500 | cBioPortal/ | |
| | CRPC that were ARSI-naive at | | of transcript | | datahub/ | |
| | biopsy | | per Million | | tree/master/ | |
| | After sample collection, | | mapped | | public/ | |
| | patients were treated with | | reads (FPKM) | | prad_su2c_2019 | |
| | Abiraterone | | | | | |
| | Use: | | | | | |
| | Negative control | | | | | |
| PROMOTE | Description: | 77 | RNA | Illumina | dbGap | PMID: 29069303 |
| | Samples from patients with | | sequencing | HiSeq 2500 | phs001141.v1.p1 | |
| | CRPPC obtained from | | | | | |
| | biopsies of metastatic tissues | | | | | |
| | After sample collection, | | | | | |
| | patients were subsequently | | | | | |
| | treated with Abiraterone for | | | | | |
| | 12 weeks. | | | | | |
| | Use: | | | | | |
| | Negative control | | | | | |

### II. Experimental Methods

Generation of Enzalutamide-resistant cell lines: LNCaP (clone FDG) and C42B cells were purchased from ATCC and were grown in RPMI1640 media (GIBCO #11875093) supplemented with 10% Fetal Bovine Serum (FBS, Corning Cat #35-011-CV) and maintained at 370° C. in and 5% CO2. Enzalutamide powder was purchased from Sellekchem (cat #S1250) and re-suspended in DMSO. Cells were plated in 6 well plates and treated either with DMSO, or with Enzalutamide (20 μM), refreshed every 4 days for up to 3 months until the resistance emerged. RNA from cells was extracted on indicated days using the methods described below.

RNA extraction, cDNA preparation, transcript knockdown, and qRT-PCR analysis: RNA was isolated from cells by the Quick-RNA miniprep kit (Zymogen #R1054) and digested with DNase (provided in the kit). cDNA was synthesized from 1 μg RNA, using an All-in-One 5×RT-master mix (Abm #G592), per the manufacturer's protocol. qRT-PCR was carried out on the StepOne Real-Time PCR system (Applied Biosystems) using gene-specific primers designed with Primer-BLAST and synthesized by IDT Technologies. ON-TARGETplus SMARTpool (cat #L005102-00-0005) was obtained from Dharmacon and was used at 100 nmol/L. Cells were transfected in 6-well plates at a density of 100,000 cells per well using Lipofectamine RNAiMax (Invitrogen #13778075), according to the manufacturer's protocol. RNA was extracted and converted to cDNA as described above. qRT-PCR data were analyzed using the relative quantification method using 18sRNA as an internal reference, and plotted as average fold-change compared with DMSO the non-targeting siRNA (Relative Quantity or RQ). Determination of transcript levels was carried out using Fast SYBR Green Master Mix (Invitrogen), using specific primer sets for c-MYC: c-MYC (F) 5′-CCTGGTGCTCCATGAGGAGAC-3′ (SEQ ID NO: 1); c-MYC (R) 5′-CAGACTCTGACCTTTTGCCAGG-3; (SEQ ID NO: 2).

Evaluating expression of AR: To evaluate the expression of AR in Enzalutamide-naïve and Enzalutamide-resistant conditions, we utilized cells from LNCaP and C42B cell lines under Enzalutamide-naïve and Enzalutamide-resistant conditions (as described above) and determined the expression level of AR under both conditions using qRT-PCR assay (described above). The specific set of primers used for AR includes: AR (F) 5′-TCTTGTCGTCTTCGGAAATGTT-3′ (SEQ ID NO: 3); AR (R) 5′-AAGCCTCTCCTTCCTCCTGTA-3′ (SEQ ID NO: 4).

Evaluating expression of NME2 in Enzalutamide-resistant vs Enzalutamide-nave cells: To evaluate the expression of NME2 in Enzalutamide-naïve and Enzalutamide-resistant conditions, we utilized cells from LNCaP and C42B cell lines under Enzalutamide-naïve and Enzalutamide-resistant conditions (as described above) and determined the expression level of NME2 under both conditions using qRT-PCR assay (as described above). The specific set of primers used for NME2 is: NME2 (F) 5′-AGGATTCCGCCTTGTTGGTCTG-3′ (SEQ ID NO: 5); NME2 (R) 5′-CGGCAAAGAATGGACGGTCCTT-3′ (SEQ ID NO: 6).

Knockdown of NME2: Two different siRNA against NME2 (siNME2 #1 AAUAAGAGGUGGACACAAC (SEQ ID NO: 7); siNME2 #2 CUGAAGAACACCUGAAGCA (SEQ ID NO: 8)), or non-targeting control (siScram) were obtained from Dharmacon and used at 100 nmol/L.

### Example 2

### Identification Role of MYC Pathway in Enzalutamide Resistance

We observed that the levels of MYC are increased in Enzalutamide-resistant conditions (presence of 20 μM Enzalutamide for up to 3 months, EnzaRes) compared to Intact (DMSO) conditions in LNCaP (a cell line derived from prostate cancer metastasis to lymph-node, commonly used to study Enzalutamide resistance) and C42B (LNCaP metastatic CRPC derivative) cell lines (FIGS. 1A-1B, one-tailed Welch t-test p-value=0.002 and p-value=0.0018 for LNCaP and C42B cells, respectively), which were accompanied by increased levels of AR (FIGS. 9A-9B, one-tailed Welch t-test p-value=0.011 and p-value=0.001 for LNCaP and C42B cells, respectively).

To evaluate if this observation could be translated to human patients, we tested if high MYC pathway activity was characteristic of CRPC patients at risk of developing resistance to Enzalutamide. For this, we utilized RNA-seq profiles of CRPC patients from Abida et al., specifically selecting samples from CRPC patients that did not receive any ARSI treatment prior to sample collection (ARS-naïve). We further selected patients that after biopsy (sample collection), were treated with Enzalutamide and monitored for Enzalutamide-associated disease progression (Table 1, n=22). We subjected this patient cohort to single-sample Gene Set Enrichment Analysis (GSEA) to estimate activity levels of MYC pathway in each patient, that were then subjected to unsupervised clustering (see Methods), separating patients with high MYC pathway activity (FIG. 1C, yellow, n=15) and normal/low MYC pathway activity (FIG. 1C, blue, n=7). We then compared Enzalutamide-associated disease progression (which was defined in Abida et al. as the time on Enzalutamide without being subjected to another agent, such as taxane) between these groups using Kaplan-Meier survival analysis and Cox proportional hazards modeling, which demonstrated a significant difference between patients from high MYC and normal/low MYC pathway activity groups (FIG. 1C, log-rank p-value=0.012, adjusted HR (hazard ratio)=4.39, CI (confidence interval): 1.2-15.97), indicating that increased MYC pathway activity is characteristic for patients with a higher risk of resistance to Enzalutamide. The patient group with high MYC pathway activity also demonstrated increased levels of AR expression and activity (see Methods) (FIGS. 9C-9D, one-tailed Welch t-test p-value=0.002 and p-value=0.01, for AR expression and AR activity, respectively) and significant correlation was observed between MYC pathway activity and AR expression/activity levels in the Abida et al. cohort (n=22), as described above (Spearman correlation rho=0.482, p-value=0.024; and Spearman correlation rho=0.484, p-value=0.023, for AR expression and AR activity, respectively).

To further evaluate if this finding was specific to Enzalutamide treatment, we tested if MYC pathway activity could predict treatment response to Abiraterone in CRPC patients. For this, we utilized RNA-seq profiles of CRPC patients from Abida et al. cohort, selecting patients that did not receive any ARSI treatment prior to sample collection and then sub-selecting patients that after biopsy (sample collection) were treated with Abiraterone and monitored for Abiraterone-associated disease progression (which was defined in Abida et al. as the time on Abiraterone without being subjected to another agent, Table 1, n=33). Contrary to the results obtained from Enzalutamide-associated disease progression, we identified no significant difference in Abiraterone-associated disease progression between patients from high MYC and normal/low MYC groups (FIG. 1D, log-rank p-value=0.23, adjusted HR=1.75, CI: 0.7-4.40). Taken together, these analyses demonstrate that increased activity of MYC pathway is indicative of a higher risk of resistance specifically to Enzalutamide and could potentially serve as a marker to stratify patients for their risk of developing resistance to Enzalutamide.

### Defining MYC Upstream Regulatory Programs Through Mechanism-Centric Network Analysis

To elucidate MYC regulation implicated in Enzalutamide resistance and define potential additional axes for salvage therapeutics, we investigated transcriptional regulatory mechanisms upstream of MYC pathway that might affect MYC while also governing Enzalutamide resistance. For this, we reconstructed a de novo CRPC-specific mechanism-centric regulatory network, which connects molecular pathways (FIG. 2A) with potential upstream transcriptional regulatory (TR) programs (FIG. 2A), through “TR-2-PATH” method. Nodes in this mechanism-centric network do not correspond to individual genes or alterations, but rather represent mechanisms: such as transcriptional regulatory programs or molecular pathways (FIG. 2A). For TR-2-PATH network reconstruction, we utilized RNA-seq profiles from CRPC patients in the Stand Up to Cancer (SU2C) East Coast cohort, excluding repeated samples and samples from Abida et al. (Table 1, n=153). The selected SU2C East Coast cohort was well-suited for CRPC mechanism-centric network reconstruction as it (i) constitutes the largest-to-date cohort of CRPC patients, essential for statistical learning/inference; (ii) is characterized by wide-ranged age (59.2±8.38 years) and prostate specific antigen (PSA) levels (234.5±1574.4 ng/ml); (iii) includes different metastatic sites, including bone (n=39), liver (n=26), lymph-node (n=57), prostate (n=4), lung (n=2), adrenal (n=1), other soft tissue (n=19), etc.; and (iv) represents different stages of therapeutic intervention, including samples from patients previously exposed to ARSIs (including Enzalutamide and Abiraterone, n=67), ARSI-naïve at the time of sample collection (n=75), currently on treatment (n=4), etc.; all together capturing a wide range of clinical variables necessary for accurate statistical inference.

To evaluate relationships between molecular pathways and their upstream transcriptional regulatory programs, we first needed to estimate pathway activity levels and transcriptional regulator activity levels in each sample in the SU2C East Coast cohort (FIG. 2A). To estimate pathway activity levels, we performed single-sample GSEA on the scaled SU2C East Coast cohort, so that each sample (n=153) was used as a reference signature and each molecular pathway (n=883, from KEGG38, BioCarta, Reactome, and Hallmark collections) was as a query. To estimate activity of TRs in the SU2C East Coast cohort, we performed VIPER analysis on the scaled SU2C East Coast cohort (as above) utilizing each sample (n=153) as a reference and transcriptional regulatory programs (n=2,678) from a prostate cancer specific interactome as a query (see Methods). For each molecular pathway, its activity level (defined as Normalized Enrichment Scores, NES) across all patients in the cohort then defined a “pathway activity vector” (FIG. 2A). Similarly, for each transcriptional regulatory program, its activity level across all patients in the cohort defined a “TR activity vector” (FIG. 2A, bottom). All pairs of TR-pathway activity vectors were then subjected to linear regression analysis (see Methods), where “TR activity vector” was utilized as a predictor (independent) variable and “pathway activity vector” was used as a response (dependent) variable, with an objective to identify TR programs whose changes could potentially explain changes in the activity of molecular pathways in CRPC-specific manner. Significant relationships, corrected for multiple hypotheses testing (see Methods), between TRs and pathways (both positive and negative) were then considered for network reconstruction (FIG. 2A).

To ensure that the network is robust to experimental and sampling noise, we enhanced our network reconstruction with bootstrap analysis. For this, patients from the SU2C East Coast cohort (n=153) were sampled with replacement (see Methods, k=100) and each bootstrap was subjected to TR-2-PATH network reconstruction. A comparison of edge distributions across the 100 bootstrapped networks showed their similarity (FIG. 2E), indicating the method's overall reproducibility. A total of 100 bootstrapped networks were then used to assign “weight” to each edge in the network reconstructed from the whole dataset (n=153), reflecting the number of times an edge appears (and thus could be recovered) across the bootstrapped networks (FIG. 2B). Unsupervised t-distributed stochastic neighbor embedding clustering (t-SNE) was utilized to cluster molecular pathways based on weights of their incoming edges, demonstrating co-clustering of MYC pathway with Chemokine, Cytokine, IL-6, JAK STAT 3 signaling, and IgA pathways (FIG. 2C), demonstrating their potential cross-talk in CRPC setting.

### Network Mining I: Identifying Differentially Altered Sub-Networks

The next step was to utilize this network to identify TR programs upstream of MYC pathway that are involved in Enzalutamide response and resistance. To achieve this, we aimed to identify parts (sub-networks) of the mechanism-centric network that significantly change (alter) between phenotypes of interest, in our case-phenotypes that describe response to Enzalutamide (FIG. 3A). To accurately capture response and resistance to Enzalutamide in a controlled setting, we utilized gene expression profiles from Kregel et al. (Table 1, n=12), which is based on the LNCaP experimental system that has been widely used to study Enzalutamide-resistance previously. These profiles included (i) LNCaP parental intact samples subjected to DMSO (phenotype 1-Intact, FIG. 3A); (ii) LNCaP samples treated for 48 hours with Enzalutamide, where their survival and proliferation was sensitive to Enzalutamide (phenotype 2-EnzaSens, FIG. 3A; and (iii) LNCaP samples treated with Enzalutamide for 6 months, where their survival and proliferation did not depend on Enzalutamide (phenotype 3-EnzaRes, FIG. 3A).

We hypothesized that regulatory programs that are active in the intact state, then are repressed by Enzalutamide treatment (EnzaSens phenotype) and further re-activated as Enzalutamide resistance develops (EnzaRes phenotype) would be effective candidates to uncover mechanisms that govern Enzalutamide-resistance (FIG. 3A). Such network mining (using pairwise phenotype comparison, FIGS. 3C-3D) identified TR mechanisms (n=28) upstream of the MYC pathway, which constitutes of MYC-centric TR sub-network with a significant “active->repressed->reactivated” activity changes across the Enzalutamide-related phenotypes (FIG. 3B).

### Network Mining II: Prioritization of Upstream Regulatory Programs

The next step was to prioritize the identified transcriptional regulatory programs upstream of a pathway of interest (e.g., MYC pathway) for experimental validation and potential salvage therapeutic targeting. We developed such a prioritization step to overcome several important drawbacks, commonly present in widely utilized statistical analyses. First of all, our method considers potential multi-collinearity among input variables (TRs), which is often naturally present in biological systems, yet can substantially obstruct statistical learning. In fact, variance inflation factor (VIF) analysis of the 28 TR programs identified upstream of MYC demonstrated significant multi-collinearity (all TRs had VIF>10, a multi-collinearity threshold suggested in Vatcheva et al. (Epidemiology (Sunnyvale) 6:227, 2016 and Kim (Korean J. Anesthesiol. 72:558-569, 2019)) (FIG. 4) and thus requires special methods to avoid information loss or model misinterpretation. Commonly utilized methods for handling multi-collinearity (e.g., regularization techniques), often keep one of the collinear variables in the model and eliminate others at random, thus limiting biological interpretability and translatability of the model. Our technique keeps all input variables intact, instead identifying their potential groups (based on their effect on the pathway of interest) and preventing information loss. Furthermore, our prioritization method not only test for direct regulatory relationships but also considers that a meaningful regulatory relationship can exist between entities that do not necessarily have direct (but rather indirect) interactions, which are widely present in biological systems, potentially including relationships between TRs and biological pathways.

To overcome these limitations, we developed a prioritization step, inspired by the Partial Least Squares (PLS) approach, which has been mostly utilized in social sciences (sometimes referred to as a “supervised PCA”) but has not been used for network-based mining in oncology to date. Briefly, to prioritize the effect of the TR programs on the MYC pathway, our approach considers TR activity vectors as predictor (input) variables and utilizes MYC pathway activity vector as a response (output) variable (FIG. 5A, left). It then regresses TR activity vectors on the MYC pathway vector so that a linear combination of TRs defines a latent variable (FIG. 5A, left). This latent variable is then “subtracted” from the TR activity vectors, leaving the residuals to be utilized for defining the next latent variable. Identified latent variables do not express collinearity or multi-collinearity and are utilized as axes to build a “circle of correlation” (FIG. 5A, middle). Such a circle of correlation depicts the association of TR programs and the MYC pathway (defined as arrows on the circle of correlation, see Methods) to each latent variable. We then defined a method that utilized unsupervised hierarchical clustering on the degree of closeness (angle) between TR and pathway arrows so that TRs in high proximity to one another (thus having similar effects on latent variables) are grouped as they express simultaneous effect on the MYC pathway (FIG. 5A, right). Such TR groups/clusters (which also include groups with one TR) are then “prioritized” based on their effect on the MYC pathway activity (FIG. 5A, right) using effect scores, which are defined as a combination of (i) degree of closeness between a TR group/cluster and the MYC pathway on the circle of correlation (angle between their arrows), which reflects effect of each TR group activity changes on MYC pathway; (ii) association (Pearson correlation) between a TR group/cluster and each evaluated latent variable, which reflects contribution of each TR group to each latent variable; and (iii) edge weight between TR group/cluster and the MYC pathway from the TR-2-PATH mechanism-centric network reconstruction step, which reflects robustness of their regulatory relationship (FIG. 5B).

This approach identified 7 TR groups/clusters, based on their effect on the MYC pathway activity (two of the clusters had single TRs, FIG. 5C): group/cluster 1 (HNRNPAB, YEATS4, BAZ1A, ZNF146, WDR77, RUVBL1, and PA2G4), group/cluster 2 (MYBBP1A), group/cluster 3 (NME2), group/cluster 4 (ACTL6A, LRPPRC and SRFBP1), group/cluster 5 (FOXM1, MYBL2, BRCA1, MLF1IP, ASF1B, ZNF367, CENPF, ZNF165, CENPK, and UHRF1), group/cluster 6 (BRCA2, PTTG1, and BLM), and group/cluster 7 (TIMELESS, TRIP13, and DNMT3B) (FIG. 5C). Each group/cluster was then assigned an effect score, with group/cluster 3 (NME2) having the highest effect (score) on the MYC pathway (FIG. 5C, FIGS. 6A-6B, Table 2). While our analysis nominated NME2 transcriptional regulatory program to have the highest effect on MYC pathway in Enzalutamide-associated CRPC context, it has also been previously shown to bind to the MYC promoter region and upregulate MYC transcription, suggesting that further investigation of this relationship may uncover aspects of the transcriptional regulatory mechanisms governing the MYC pathway which could potentially provide an additional axis for therapeutic targeting for CRPC patients.

TABLE 2
Prioritization of transcriptional regulatory programs affecting MYC pathway

| clusters of | correlation of | | correlation of | | | | | | |
|---|---|---|---|---|---|---|---|---|---|
| collinear/stand alone | transcriptional | | transcriptional | | angle | | | | |
| transcriptional | regulatory | | regulatory | | with | | | | |
| regulatory | programs with | | programs with | | respect | | | | |
| programs affecting | latent variable | | latent variable | | to MYC | | edge | | rank |
| MYC pathway | 1 | rank | 2 | rank | pathway | rank | weight | rank | product |
| Cluster 3 (NME2) | 0.52 | 6 | 0.53 | 1 | 4.18 | 1 | 100.00 | 1 | 1.57 |
| Cluster 5 | 0.89 | 1 | −0.35 | 6 | 19.76 | 4 | 100.00 | 1 | 2.21 |
| Cluster 1 | 0.63 | 4 | −0.10 | 5 | 10.86 | 2 | 97.14 | 2 | 2.99 |
| Cluster 7 | 0.87 | 2 | −0.49 | 7 | 34.83 | 7 | 100.00 | 1 | 3.15 |
| Cluster 6 | 0.84 | 3 | 0.18 | 4 | 11.02 | 3 | 96.67 | 3 | 3.22 |
| Cluster 2 (MYBBP1A) | 0.16 | 7 | 0.38 | 3 | 26.37 | 6 | 100.00 | 1 | 3.35 |
| Cluster 4 | 0.57 | 5 | 0.38 | 2 | 23.69 | 5 | 91.67 | 4 | 3.76 |

### Example 3

### Validation in Clinical Cohorts and Enzalutamide-Specificity

We next sought to confirm and evaluate if activation of NME2 TR program and MYC pathway are present in patients at risk of resistance to Enzalutamide and if they can be used as markers to risk-stratify patients prior to Enzalutamide administration. First, to evaluate if activity of the MYC pathway and NME2 TR program are high in treatment-naïve patients, who are at the risk of developing resistance to Enzalutamide, we have evaluated single-cell profiles from two sequential samples from a CRPC patient (01115655) that eventually failed Enzalutamide: neoadjuvant sample (prior to Enzalutamide treatment, FIG. 8A) and adjuvant sample (after developing resistance to Enzalutamide, EnzaRes, FIG. 8B) from He et al. (Table 1). After subjecting single-cell transcriptomic data to unsupervised uniform manifold approximation and projection (UMAP) clustering, to identify adenocarcinoma cells among the cell populations we assessed activity levels of AR, alongside expression of CK8 and CD45 (FIGS. 7A-7D). Following adenocarcinoma identification, we evaluated activity levels of the MYC pathway and NME2 TR program in both neoadjuvant and adjuvant samples. Our analysis indicated significantly higher levels of both NME2 TR activity and MYC pathway activity in adenocarcinoma cells, compared to other cells in Enzalutamide-naïve (neoadjuvant, FIG. 8A, one-tailed Welch t-test p-value <2.26E-16 for NME2 and p-value=1.74E-7 for MYC) and Enzalutamide-resistant, EnzaRes (adjuvant, FIG. 8B, one-tailed Welch t-test p-value <2.26E-16 for NME2 and p-value <2.26E-16 for MYC) samples, indicating that (i) both high-MYC pathway and high-NME2 TR activity levels were present prior to treatment in a patient who was at risk of developing subsequent resistance to Enzalutamide, nominating them as markers to identify patients at potential risk of Enzalutamide resistance; and (ii) both high-MYC pathway and high-NME2 TR activity levels were also observed after resistance to Enzalutamide developed (similar to our observation in LNCaP and C42B cell lines, FIGS. 1A-1B, FIGS. 6A-6B), cautiously nominating a MYC-centered salvage therapeutic line for patients that fail Enzalutamide.

Given increased activity levels of both NME2 TR and MYC pathway in a single-cell sample from a treatment-naïve patient that later developed resistance to Enzalutamide, we sought to confirm their collective ability to predict CRPC patients at the treatment-naïve stage for risk of developing primary resistance to Enzalutamide using the Abida et al. cohort, which was also utilized in FIG. 1C (Table 1, n=22). As previously described, we used ARSI-naïve CRPC patients (that were later subjected to Enzalutamide and monitored for Enzalutamide-associated disease progression) to estimate NME2 TR and MYC pathway activity levels in each patient (see Methods). The NME2 TR and MYC pathway demonstrated striking concordance of their activity levels (FIG. 8C, left top, Pearson r=0.8, p-value=5.2E-6). We then subjected the activity levels of the NME2 TR and MYC pathway to unsupervised clustering (see Methods) that identified (i) patients with both high levels of NME2 transcriptional activity and MYC pathway activity (FIG. 8C, left bottom, n=13) and (ii) patients with at least one low/normal NME2 transcriptional activity and/or MYC pathway activity, categorized as “others” (FIG. 8C, left bottom, n=9). A comparison of these groups using Kaplan-Meier survival analysis and Cox proportional hazards model analysis, demonstrated a significant difference in their Enzalutamide-associated disease progression (FIG. 8C, right, log-rank p-value=0.0035, adjusted HR=5.28, CI=1.58-18.38, see Methods), suggesting that the high activity levels of NME2 TR and MYC pathway can be utilized to predict CRPC patients who are at a higher risk of developing resistance to Enzalutamide, prior to therapy administration. Next, to evaluate if our analysis is more applicable to specific patient sub-groups, we performed a stratified Kaplan-Meier survival analysis and separated patients by age at diagnosis (median age ≤57 and >57), age at biopsy (median age ≤66.3 and >66.3), and Gleason score (Gleason score 6+7 and 8+9) (FIGS. 11A-11F), and demonstrated that predictive ability of NME2 TR and MYC pathway was applicable in all stratified patient groups and was not significantly affected by these co-variates.

To extend our validation studies to an additional patient cohort, we utilized SU2C West Coast cohort (Table 1, n=83) which comprises of CRPC patients that were subjected to Enzalutamide and/or Abiraterone (complete separation of treatments was not possible according to correspondence with the dataset owners) either before or after biopsy (before or after sample collection) and were subsequently monitored for treatment-associated disease progression (which was defined in Quigley et al. and Aggrawal et al. as increase in PSA level (minimum 2 ng/mL) that has risen at least twice, in an interval of at least one week or soft tissue progression (nodal and visceral) based on RECIST v1.1). For each of these CRPC patients, we first estimated their NME2 TR and MYC pathway activity levels (see Methods) followed by evaluating association between NME2 TR and MYC pathway activity. Our analysis demonstrated striking concordance between NME2 TR and MYC pathway activity (FIG. 8D, left top, Pearson r=0.82, p-value <2.2E-16, see Methods). Next, we subjected the activity levels of the NME2 TR and MYC pathway to unsupervised clustering that identified (i) patients with both high NME2 TR and MYC pathway activity levels (FIG. 8D, left bottom, n=40) and (ii) patients with at least one low/normal NME2 TR and/or MYC activity levels, categorized as “others” (FIG. 8D, left bottom, n=43). A comparison of these groups using Kaplan-Meier survival analysis and Cox proportional hazards model analysis demonstrated a significant difference in treatment-associated disease progression (FIG. 8D, right, log-rank p-value=0.026, adjusted HR=1.90, CI=1.11-3.24, see Methods), which supports our previous observations.

To further investigate and confirm that the activation of NME2 TR and MYC pathway could specifically predict Enzalutamide-failure (and not, for example, Abiraterone-failure), we analyzed RNA-seq profiles from two Abiraterone-specific cohorts: (i) ARSI-naïve CRPC patients from Abida et at. that were subjected to Abiraterone after sample collection and monitored for Abiraterone-associated disease progression, as in FIG. 1D (Table 1, n=33) and (ii) PROMOTE cohort, which is comprised of ARSI-naïve CRPC patients, subjected to Abiraterone for 12 weeks after sample collection and then assessed for disease progression (binary outcomes, where disease progression was defined based on the combined score that included serum PSA level, bone and CT imaging and symptom assessments at week 12, Table 1, n=77). In both cohorts, we estimated the NME2 TR and MYC pathway activity in each sample and subjected them to similar analyses as above. Kaplan-Meier survival analysis36 and Cox proportional hazards model analysis on the Abida et al. cohort demonstrated no significant difference in Abiraterone-associated disease progression between the two identified patient groups (FIG. 8E left, log-rank p-value=0.09, adjusted HR=2.37, CI=0.92-6.09). ROC analysis in the PROMOTE cohort demonstrated that activation of NME2 and MYC did not classify patients based on their binary response to Abiraterone (FIG. 8E, right, AUROC=0.58, where AUROC=0.5 indicates a random classifier), demonstrating that partnership between NME2 TR and MYC pathway is specifically indicative of the risk of developing resistance to Enzalutamide and not Abiraterone.

### Example 4

### Comparison to Common Markers of PCa Aggressiveness and Treatment Response

We next compared the ability of NME2 TR and MYC pathway to predict Enzalutamide resistance in Abida et al. cohort, with the predictive ability of known markers of prostate cancer (i) aggressiveness, (ii) response to first-generation ADT and ARSIs (not specific to any particular drug), and (iii) Enzalutamide-specific response (FIGS. 12A-12F). Transcriptomic and genomic markers were considered separately. Comparison to transcriptomic markers of PCa aggressiveness (FIG. 12A), demonstrated that three markers (ONECUT2, ERG, and DLX1) showed significant differential expression in the high-MYC and high-NME2 group, compared to the rest of the patients, “others” (two-tailed Welch t-test p-value <0.05, Table 3). Interestingly, all three have a direct relationship to MYC: ONECUT2 is a known transcriptional target of MYC, ERG fusion (which eventually leads to overexpression of ERG) was shown to be correlated to MYC expression, and DLX1 is a known transcriptional target of ERG (FIG. 12A). To assess an independent association of the transcriptomic markers to Enzalutamide resistance (independent of MYC and NME2), we have performed a univariable Cox proportional hazards model analysis, using Enzalutamide-associated disease progression as the end-point in Abida et al. cohort, which showed no significant association of these markers to Enzalutamide resistance (Table 3). From genomic markers of PCa aggressiveness (FIG. 12B, where TP53 and RBI have also been shown to be markers of response to ARSIs), RBI with shallow and deep deletion was significantly enriched in patients with high-NME2 and high-MYC pathway activity (Fisher's exact test p-value=0.03) (FIG. 12B, Table 4). Interestingly, RBI loss has been shown to correlate with MYC expression in small cell lung carcinoma, indicating potential cross-talk with the MYC pathway. Independent Cox proportional hazards model analysis identified a shallow and deep deletion of KRAS to be significantly associated with response to Enzalutamide (Wald p-value=0.01, Table 4), which has also been previously shown to be associated with MYC.

Comparison to transcriptomic markers of first-generation ADT and ARSIs (taken from Panja et al. (EBioMedicine 31:110-121, 2018), Arriaga et al. (Nature Cancer 1:1082-1096, 2020), Hankey et al. (Cancer Research 80:2427-2436, 2020), and Zhang et al. (Cancer Cell 38:279-296, 2020)), demonstrated that five markers (TTC27, WDR12, AZIN1, FOXA1, and GATA2) were significantly differentially expressed in the high-MYC and high-NME2 patients (two-tailed Welch t-test p-value <0.05, Table 5, FIG. 12C). Interestingly, WDR12 and AZIN1 are members of the MYC pathway and TTC27, FOXA1, and GATA2 are MYC transcriptional targets. Furthermore, Cox proportional hazards model analysis37 indicated that five of the transcriptomic markers (STMN1, WDR12, AZIN1, MAD2LI, and MCM4) had a significant association with response to Enzalutamide (Wald p-value <0.05, Table 5), yet many of them were borderline significant and did not outperform MYC and NME2 (Table 5). Additionally, genomic markers of first-generation ADT and ARSIs described in Arriaga et al. and Abida et al., had no significant enrichment in the high-MYC and high-NME2 group (FIG. 12D, Table 6) or independent response to Enzalutamide.

Comparison to transcriptomic markers of Enzalutamide-specific response (described by Zhang et al (Cancer Cell 38:584-598, 2020), Kohrt et al. (Mol. Cancer Ther. 20:398-409, 2021), Verma et al. (Int. J. Mol. Sci. 21, doi: 10.3390/ijms21249568, 2020), He et al. (Nucleic Acids Res. 46:1895-1911, 2018), Korpal et al. (Cancer Discov. 3:1030-1043, 2013), Taavitsainen et al. (Nature Commun. 12:5307, 2021), in addition to others) demonstrated that a group of 14 markers (EIF6, AR, SOX9, TK1, PPP1R14B, TMEM54, UBE2S, MYC, ODC1, DYNLL1, CD81, BCL2, TCF4 and RAC1) had significant differential expression in the high-MYC and high-NME2 group (two-tailed Welch t-test p-value <0.05, Table 7, FIG. 12E). Interestingly, 12 of these (EIF6, SOX9, TK1, PPP1R14B, TMEM54, UBE2S, MYC, ODC1, DYNLL1, CD81, BCL2, and TCF4) were transcriptional MYC targets, determined from ChEA transcription factor targets dataset. Another member of this group, AR, as we have shown previously (FIG. 9C), was also differentially expressed between the groups. Finally, RAC1 (FIG. 12E, Table 7) is a member of the RAS pathway which has been shown to be associated with MYC pathway in CRPC samples. Cox proportional hazards model analysis37 demonstrated that 10 of the transcriptomic markers (EIF6, ACAT1, TK1, PPP1R14B, TMEM54, UBE2S, DYNLL1, TUBA1C, RAC1, and WNT5A) had significant association with response to Enzalutamide (Wald p-value <0.05, Table 7), yet many of them were border-line significant and none of them outperformed NME2 and MYC (Table 7). None of the genomic markers of Enzalutamide-specific response (described by Zhang et al. and Guan et al. (Clin Cancer Res 26:3616-4624, 2020)) were significantly enriched in the high-MYC and high-NME2 group (FIG. 12F, Table 8) or independently associated with response to Enzalutamide. Taken together, these findings indicate that the majority of the markers of PCa aggressiveness and therapeutic response that are enriched in the high-MYC and high-NME2 group are associated with MYC-related mechanisms and none of them outperform the ability of MYC and NME2 to predict risk of Enzalutamide resistance.

TABLE 3
Comparison to known markers of overall prostate cancer aggressiveness

| | | | Comparison of | | | | | |
|---|---|---|---|---|---|---|---|---|
| | | | known markers | | Association of known markers to | | | |
| | Progression | | between groups | | Enzalutamide associated progression | | | |
| Human | related | Publication | T-test | | Hazard | | | |
| entrez | markers | PubMed Id | tvalue | T-test p | ratio | lower_95 | upper_95 | Cox p |
| 9480 | ONECUT2 | PMID: 30478421 | 2.93 | 0.01* | 1.15 | 0.72 | 1.83 | 0.57 |
| 2078 | ERG | PMID: 16254181, | 2.41 | 0.03* | 1.00 | 0.99 | 1.02 | 0.78 |
| | | PMID: 19584279 | | | | | | |
| 1745 | DLX1 | PMID: 34493733 | 2.13 | 0.05* | 1.00 | 0.96 | 1.04 | 0.98 |
| 3579 | CXCR2 | PMID: 31801883 | −1.69 | 0.13 | 0.98 | 0.95 | 1.02 | 0.38 |
| 2146 | EZH2 | PMID: 34489575 | −1.33 | 0.21 | 1.01 | 0.95 | 1.08 | 0.78 |
| 4824 | NKX3-1 | PMID: 11085535 | 1.24 | 0.24 | 1.00 | 1.00 | 1.00 | 0.48 |
| 196528 | ARID2 | PMID: 34163028 | 0.68 | 0.50 | 0.96 | 0.86 | 1.09 | 0.55 |
| 6597 | SMARCA4 | PMID: 33144576 | 0.59 | 0.56 | 0.97 | 0.93 | 1.02 | 0.22 |
| 2305 | FOXM1 | PMID: 30719174 | 0.45 | 0.66 | 1.06 | 0.99 | 1.13 | 0.07 |
| 2026 | ENO2 | PMID: 31547070 | 0.35 | 0.73 | 1.03 | 0.97 | 1.09 | 0.33 |
| 7113 | TMPRSS2 | PMID: 19584279 | 0.18 | 0.86 | 1.00 | 1.00 | 1.00 | 0.77 |
| 1869 | E2F1 | PMID: 31802906 | 0.17 | 0.87 | 1.08 | 0.99 | 1.18 | 0.10 |
| 6595 | SMARCA2 | PMID: 33144576 | 0.09 | 0.93 | 0.95 | 0.87 | 1.03 | 0.23 |

*significant p-value < 0.05

TABLE 4
Comparison to known genomic markers of overall prostate cancer aggressiveness

| | | | Deletion (both Shallow and Deep) | | | | | Gain and Amplification |
|---|---|---|---|---|---|---|---|---|
| | Progression | | Fisher | | | | | Fisher |
| Human | related | Publication PubMed | exact | Hazard | lower_{—} | upper_{—} | | exact |
| entrez | markers | Id | p | ratio | 95 | 95 | Cox p | p |
| 324 | APC | PMID: 26000489, | 0.33 | 1.24 | 0.43 | 3.57 | 0.68 | 1 |
| 1499 | CTNNB1 | PMID: 29610475, | 1 | NA | NA | NA | NA | 1 |
| 5290 | PIK3CA | PMID: 30928160 | 1 | NA | NA | NA | NA | 1 |
| 472 | ATM | | 0.15 | 1.3 | 0.29 | 5.81 | 0.72 | 0.11 |
| 675 | BRCA2 | | 0.36 | 1.09 | 0.35 | 3.38 | 0.87 | 0.24 |
| 51755 | CDK12 | | 1 | 9.7E+09 | 0 | Inf | 0.99 | 0.62 |
| 5728 | PTEN | | 1 | 1.33 | 0.51 | 3.49 | 0.55 | 1 |
| 7157 | TP53 | | 0.09 | 2.1 | 0.79 | 5.57 | 0.13 | 1 |
| 5925 | RB1 | | 0.03* | 1.89 | 0.69 | 5.18 | 0.21 | 1 |
| 3169 | FOXA1 | | 1 | 0.64 | 0.08 | 4.94 | 0.67 | 1 |
| 3845 | KRAS | PMID: 26299074 | 0.62 | 5.48 | 1.5 | 19.93 | 0.01* | 1 |
| 6776 | STAT5A | PMID: 31292140 | 0.24 | 3.34 | 0.85 | 0.001 | 0.08 | 0.41 |

| | | | | | Diploid | | | | |
|---|---|---|---|---|---|---|---|---|---|
| | Gain and Amplification | | | | Fisher | | | | |
| Human | Hazard | lower_{—} | upper_{—} | Cox | exact | Hazard | lower_{—} | upper_{—} | |
| entrez | ratio | 95 | 95 | p | p | ratio | 95 | 95 | Cox p |
| 324 | 0.64 | 0.08 | 4.94 | 0.67 | 0.16 | 0.94 | 0.34 | 2.55 | 0.9 |
| 1499 | 0.76 | 0.21 | 2.66 | 0.66 | 1 | 1.31 | 0.37 | 4.61 | 0.66 |
| 5290 | 1.42 | 0.54 | 3.78 | 0.47 | 1 | 0.69 | 0.26 | 1.85 | 0.47 |
| 472 | 1.94 | 0.62 | 5.99 | 0.24 | 1 | 0.54 | 0.19 | 1.48 | 0.23 |
| 675 | 1.46 | 0.41 | 5.12 | 0.55 | 0.07 | 0.75 | 0.28 | 2 | 0.57 |
| 51755 | 0.71 | 0.2 | 2.51 | 0.59 | 0.36 | 0.98 | 0.31 | 3.06 | 0.97 |
| 5728 | 0.64 | 0.08 | 4.94 | 0.67 | 0.67 | 0.83 | 0.31 | 2.21 | 0.72 |
| 7157 | NA | NA | NA | NA | 0.09 | 0.47 | 0.17 | 1.25 | 0.13 |
| 5925 | NA | NA | NA | NA | 0.03* | 0.52 | 0.19 | 1.44 | 0.21 |
| 3169 | 0.72 | 0.16 | 3.19 | 0.67 | 0.62 | 1.5 | 0.42 | 5.29 | 0.52 |
| 3845 | 2.48 | 0.52 | 11.86 | 0.25 | 1 | 0.16 | 0.04 | 0.59 | 0.005* |
| 6776 | 1.20E−08 | 0 | Inf | 0.99 | 0.62 | 0.9 | 0.25 | 3.16 | 0.87 |

TABLE 5
Comparison to known markers related with response to ADT and ARSI

| | | | Comparison of | | | | | |
|---|---|---|---|---|---|---|---|---|
| | Second | | known markers | | Association of known markers to | | | |
| | generation | | between groups | | Enzalutamide associated progression | | | |
| Human | related | Publication | T-test | | Hazard | lower_{—} | upper_{—} | |
| entrez | markers | PubMed Id | tvalue | T-test p | ratio | 95 | 95 | Cox p |
| 55622 | TTC27 | PMID: 29685789 | 2.21 | 0.04* | 1.14 | 0.98 | 1.34 | 0.09 |
| 3925 | STMN1 | | 1.76 | 0.09 | 1.02 | 1.01 | 1.03 | 0.002* |
| 8468 | FKBP6 | | −1.00 | 0.35 | 0.00 | 0.00 | 296.57 | 0.29 |
| 10675 | CSPG5 | | 0.88 | 0.39 | 1.11 | 0.95 | 1.29 | 0.20 |
| 2354 | FOSB | | 0.79 | 0.45 | 1.00 | 0.96 | 1.05 | 1.00 |
| 55759 | WDR12 | PMID: 34085047 | 2.71 | 0.01* | 1.27 | 1.06 | 1.52 | 0.01* |
| 51582 | AZIN1 | | 2.54 | 0.02* | 1.02 | 1.00 | 1.03 | 0.03* |
| 6873 | TAF2 | | 1.76 | 0.10 | 1.02 | 0.95 | 1.10 | 0.60 |
| 5885 | RAD21 | | 1.67 | 0.11 | 1.01 | 1.00 | 1.02 | 0.12 |
| 11169 | WDHD1 | | −1.09 | 0.30 | 1.05 | 0.88 | 1.26 | 0.58 |
| 29028 | ATAD2 | | 0.74 | 0.47 | 1.05 | 1.00 | 1.10 | 0.06 |
| 4085 | MAD2L1 | | 0.71 | 0.49 | 1.13 | 1.04 | 1.24 | 0.01* |
| 7112 | TMPO | | −0.69 | 0.50 | 1.01 | 0.98 | 1.05 | 0.48 |
| 4173 | MCM4 | | 0.55 | 0.59 | 1.03 | 1.00 | 1.05 | 0.03* |
| 7153 | TOP2A | | −0.42 | 0.68 | 1.01 | 0.99 | 1.02 | 0.32 |
| 29127 | RACGAP1 | | −0.35 | 0.74 | 1.03 | 0.99 | 1.0 | 0.15 |
| 6732 | SRPK1 | | 0.31 | 0.76 | 1.01 | 0.98 | 1.04 | 0.46 |
| 10635 | RAD51AP1 | | −0.23 | 0.82 | 1.07 | 0.98 | 1.16 | 0.11 |
| 9134 | CCNE2 | | 0.14 | 0.89 | 1.07 | 0.93 | 1.22 | 0.34 |
| 54821 | ERCC6L | | −0.13 | 0.90 | 1.32 | 0.87 | 2.01 | 0.19 |
| 3169 | FOXA1 | PMID: 32094298 | 2.88 | 0.01* | 1.00 | 1.00 | 1.01 | 0.22 |
| 2624 | GATA2 | | 2.82 | 0.011* | 1.01 | 0.99 | 1.02 | 0.42 |
| 10481 | HOXB13 | | 1.64 | 0.13 | 1.00 | 1.00 | 1.01 | 0.23 |
| 3084 | NRG1 | PMID: 32679108 | −1.29 | 0.23 | 0.48 | 0.08 | 3.03 | 0.43 |

*significant p-value < 0.05

TABLE 6
Comparison to known genomic markers related with response to ARSI

| | | | Deletion (both Shallow and Deep) | | | | | Gain and Amplification |
|---|---|---|---|---|---|---|---|---|
| | Progression | | Fisher | | | | | Fisher |
| Human | related | Publication | exact | Hazard | lower_{—} | upper_{—} | Cox | exact |
| entrez | markers | PubMed Id | p | ratio | 95 | 95 | p | p |
| 4609 | MYC | PMID: 34085047 | 1 | NA | NA | NA | NA | 0.07 |
| 8405 | SPOP | PMID: 31061129 | 1 | NA | NA | NA | NA | 0.62 |
| 367 | AR | | 1 | NA | NA | NA | NA | 0.38 |

| | | | | | Diploid | | | | |
|---|---|---|---|---|---|---|---|---|---|
| | Gain and Amplification | | | | Fisher | | | | |
| Human | Hazard | lower_{—} | upper_{—} | Cox | exact | Hazard | lower_{—} | upper_{—} | |
| entrez | ratio | 95 | 95 | p | p | ratio | 95 | 95 | Cox p |
| 4609 | 1.79 | 0.58 | 5.52 | 0.3 | 0.07 | 0.55 | 0.18 | 1.7 | 0.3 |
| 8405 | 1.04 | 0.28 | 3.56 | 0.98 | 0.62 | 0.98 | 0.28 | 3.46 | 0.98 |
| 367 | 1.45 | 0.55 | 4.05 | 0.42 | 0.38 | 0.66 | 0.24 | 1.81 | 0.42 |

TABLE 7
Comparison to known markers related with response to Enzalutamide

| | | | Comparison of | | | | | |
|---|---|---|---|---|---|---|---|---|
| | | | known markers | | Association of known markers to | | | |
| | Enzalutamide | | between groups | | Enzalutamide associated progression | | | |
| Human | related | Publication | T-test | | Hazard | lower_{—} | | |
| entrez | markers | PubMed Id | tvalue | T-test p | ratio | 95 | upper_95 | Cox p |
| 2908 | NR3C1 | PMID: | −1.62 | 0.12 | 1.01 | 0.93 | 1.10 | 0.77 |
| 7025 | NR2F1 | 32220301 | −1.42 | 0.18 | 0.82 | 0.67 | 1.01 | 0.06 |
| 1105 | CHD1 | | −1.32 | 0.22 | 0.94 | 0.80 | 1.11 | 0.47 |
| 5454 | POU3F2 | | −0.79 | 0.45 | 0.01 | 0.00 | 131.03 | 0.31 |
| 6909 | TBX2 | | 0.1 | 0.92 | 0.99 | 0.90 | 1.10 | 0.92 |
| 3692 | EIF6 | PMID: | 3.41 | 0.003* | 1.03 | 1.01 | 1.05 | 0.003* |
| 367 | AR | 33298586 | 2.9 | 0.01* | 1.00 | 1.00 | 1.01 | 0.05 |
| 25803 | SPDEF | | 1.86 | 0.08 | 1.00 | 1.00 | 1.00 | 0.98 |
| 5718 | PSMD12 | | 1.66 | 0.12 | 1.02 | 1.00 | 1.05 | 0.11 |
| 4296 | MAP3K11 | | 1.41 | 0.18 | 1.03 | 0.97 | 1.08 | 0.34 |
| 38 | ACAT1 | | 1.32 | 0.21 | 1.03 | 1.01 | 1.04 | 0.01* |
| 2566 | GABRG2 | | −1.03 | 0.33 | 0.00 | 0.00 | 249624.90 | 0.41 |
| 7022 | TFAP2C | | 0.87 | 0.4 | 0.98 | 0.90 | 1.07 | 0.63 |
| 11140 | CDC37 | | 0.81 | 0.43 | 1.00 | 0.99 | 1.01 | 0.84 |
| 790 | CAD | | −0.2 | 0.85 | 0.97 | 0.83 | 1.13 | 0.66 |
| 1290 | COL5A2 | | −0.14 | 0.89 | 1.00 | 0.97 | 1.03 | 0.85 |
| 6662 | SOX9 | PMID: | 2.46 | 0.03* | 1.00 | 0.98 | 1.02 | 0.99 |
| 79923 | NANOG | 33339129 | −1.23 | 0.26 | 0.00 | 0.00 | 163.01 | 0.15 |
| 650 | BMP2 | | −1.11 | 0.28 | 0.89 | 0.59 | 1.34 | 0.59 |
| 960 | CD44 | | −0.99 | 0.34 | 0.99 | 0.96 | 1.01 | 0.27 |
| 5460 | POU5F1 | | −0.73 | 0.49 | 0.90 | 0.68 | 1.19 | 0.47 |
| 648 | BMI1 | | 0.5 | 0.62 | 1.01 | 0.98 | 1.05 | 0.46 |
| 6657 | SOX2 | | −0.46 | 0.65 | 0.86 | 0.64 | 1.14 | 0.29 |
| 216 | ALDH1A1 | | 0.31 | 0.76 | 1.00 | 1.00 | 1.00 | 0.92 |
| 3397 | ID1 | PMID: 33750801 | 0.55 | 0.59 | 1.00 | 0.97 | 1.02 | 0.91 |
| 51523 | CXXC5 | | 0.52 | 0.61 | 1.02 | 0.96 | 1.08 | 0.51 |
| 7083 | TK1 | PMID: | 2.76 | 0.01* | 1.03 | 1.01 | 1.05 | 0.01* |
| 1719 | DHFR | 23842682 | −0.46 | 0.66 | 1.00 | 0.97 | 1.04 | 0.81 |
| 26472 | PPP1R14B | PMID: 34489465 | 3.42 | 0.004* | 1.01 | 1.00 | 1.03 | 0.02* |
| 113452 | TMEM54 | | 3.27 | 0.004* | 1.02 | 1.01 | 1.04 | 0.01* |
| 27338 | UBE2S | | 3.3 | 0.004* | 1.06 | 1.02 | 1.10 | 0.0031* |
| 4609 | MYC | | 3 | 0.01* | 1.01 | 1.00 | 1.02 | 0.16 |
| 4953 | ODC1 | | 2.53 | 0.03* | 1.00 | 1.00 | 1.00 | 0.28 |
| 8655 | DYNLL1 | | 2.3 | 0.03* | 1.03 | 1.02 | 1.05 | 0.0002* |
| 975 | CD81 | | 2.15 | 0.05* | 1.01 | 1.00 | 1.01 | 0.11 |
| 84674 | CARD6 | | −2.11 | 0.07 | 0.91 | 0.74 | 1.12 | 0.39 |
| 4288 | MKI67 | | −1.81 | 0.11 | 0.99 | 0.95 | 1.03 | 0.71 |
| 84790 | TUBA1C | | 1.35 | 0.19 | 1.01 | 1.00 | 1.03 | 0.04* |
| 1063 | CENPF | | −1.36 | 0.21 | 1.00 | 0.95 | 1.06 | 0.97 |
| 3161 | HMMR | | −1.32 | 0.22 | 1.02 | 0.97 | 1.07 | 0.47 |
| 9787 | DLGAP5 | | −1.26 | 0.24 | 1.00 | 0.97 | 1.03 | 0.92 |
| 9793 | CKAP5 | | 1.15 | 0.27 | 1.04 | 0.99 | 1.08 | 0.13 |
| 1062 | CENPE | | −1.13 | 0.29 | 1.01 | 0.91 | 1.12 | 0.79 |
| 9232 | PTTG1 | | −0.66 | 0.52 | 1.01 | 1.00 | 1.02 | 0.14 |
| 9918 | NCAPD2 | | −0.58 | 0.57 | 1.00 | 0.97 | 1.04 | 0.89 |
| 891 | CCNB1 | | 0.54 | 0.59 | 1.03 | 1.00 | 1.05 | 0.05 |
| 991 | CDC20 | | −0.4 | 0.7 | 1.01 | 0.99 | 1.04 | 0.31 |
| 55814 | BDP1 | | 0.28 | 0.78 | 1.00 | 0.90 | 1.11 | 0.99 |
| 9133 | CCNB2 | | −0.27 | 0.79 | 1.02 | 0.99 | 1.06 | 0.24 |
| 23198 | PSME4 | | −0.18 | 0.86 | 0.98 | 0.91 | 1.06 | 0.65 |
| 5347 | PLK1 | | 0.12 | 0.91 | 1.04 | 0.99 | 1.10 | 0.09 |
| 6925 | TCF4 | PMID: 31536510 | −2.72 | 0.02* | 0.89 | 0.70 | 1.14 | 0.37 |
| 596 | BCL2 | PMID: 31228231, | −2.71 | 0.02* | 0.79 | 0.57 | 1.09 | 0.15 |
| | | PMID: | | | | | | |
| | | 33542074 | | | | | | |
| 5879 | RAC1 | PMID: 32782617 | 2.45 | 0.02* | 1.02 | 1.01 | 1.04 | 0.003* |
| 10413 | YAP1 | PMID: 33664454 | 1.34 | 0.2 | 1.01 | 0.97 | 1.05 | 0.58 |
| 7474 | WNT5A | https://doi.org/ | 0.71 | 0.49 | 1.02 | 1.00 | 1.03 | 0.02* |
| | | 10.1097/ | | | | | | |
| | | JU.0000000000002526.02 | | | | | | |
| 4853 | NOTCH2 | PMID: | 0.66 | 0.52 | 0.99 | 0.92 | 1.06 | 0.74 |
| | | 30940724 | | | | | | |
| 3551 | IKBKB | PMID: | −0.47 | 0.64 | 1.01 | 0.96 | 1.06 | 0.78 |
| | | 33542074 | | | | | | |
| 8644 | AKR1C3 | PMID: 25649766 | −0.27 | 0.79 | 1.02 | 0.99 | 1.06 | 0.17 |
| 2535 | FZD2 | https://doi.org/ | 0.01 | 1 | 0.96 | 0.66 | 1.40 | 0.84 |
| | | 10.1097/ | | | | | | |
| | | JU.0000000000002526.02 | | | | | | |

*significant p-value < 0.05

TABLE 8
Comparison to known genomic markers related with response to Enzalutamide

| | | | Deletion (both Shallow and Deep) | | | | | Gain and Amplification |
|---|---|---|---|---|---|---|---|---|
| | Progression | | Fisher | | | | | Fisher |
| Human | related | Publication | exact | Hazard | lower_{—} | upper_{—} | | exact |
| entrez | markers | PubMed Id | p | ratio | 95 | 95 | Cox p | p |
| 1105 | CHD1 | PMID: 32220301 | 0.38 | 1.05 | 0.38 | 2.92 | 0.91 | 1 |
| 54894 | RNF43 | PMID: 32727885 | 0.49 | 1.27 | 0.28 | 5.74 | 0.76 | 1 |

| | | | | | Diploid | | | | |
|---|---|---|---|---|---|---|---|---|---|
| | Gain and Amplification | | | | Fisher | | | | |
| Human | Hazard | lower_{—} | upper_{—} | | exact | Hazard | lower_{—} | upper_{—} | |
| entrez | ratio | 95 | 95 | Cox p | p | ratio | 95 | 95 | Cox p |
| 1105 | 0.64 | 0.08 | 4.94 | 0.67 | 0.2 | 1.07 | 0.4 | 2.85 | 0.88 |
| 54894 | 0.42 | 0.05 | 3.22 | 0.4 | 0.62 | 1.34 | 0.38 | 4.71 | 0.64 |

### Example 5

### Comparison to Gene-Centric Computational Methods

To evaluate if TR-2-PATH mechanism-centric predictions (activity levels of NME2 TR and MYC pathway) outperform predictive ability of commonly used gene-centric methods, we compared TR-2-PATH to differential expression analyses, Random (survival) Forest (RF), and Support Vector Machine (SVM) methods all utilized on the Enzalutamide-specific Abida et al. cohort. For differential gene expression analysis, we considered genes that were differentially expressed between the three phenotypes (Intact, EnzaSens, and EnzaRes) in the mining step I and considered genes at (i) Welch t-test p-value <0.05; (ii) top 470 differentially expressed genes (comparable to the total number of target genes and pathway genes used for activity estimation) and not excluding target/pathway genes from NME2 TR and MYC pathway; (iii) top 470 differentially expressed genes, excluding target/pathway genes from NME2 TR and MYC pathway. For RF and SVM analysis, we utilized 470 genes from (iii) to avoid overfitting and then selected top 10 most significant genes/features from the outputs. Final gene list from each of these analyses was subjected to Kaplan-Meier survival analysis and Cox proportional hazards model analysis (crude and adjusted for age and Gleason), which did not show a significant association with Enzalutamide-associated disease progression using log-rank test, Wald test, or crude/adjusted hazards models (FIGS. 10A-10D). Such analysis demonstrates that mechanisms identified by TR-2-PATH (NME2 TR and MYC pathway) have significant advantage in predicting the risk of Enzalutamide resistance, compared to commonly used gene-centric methods.

### Example 6

### Targeting MYC and NME2 is Beneficial in Enzalutamide Resistant Conditions

Given that NME2 and MYC are upregulated in both patients that are at risk of Enzalutamide resistance and patients that fail Enzalutamide, we experimentally evaluated the benefits of therapeutic targeting of MYC and NME2 in similar experimental conditions. For this, we utilized LNCaP and C42B cell lines, as our experimental systems in Enzalutamide-naïve and Enzalutamide-resistant (EnzaRes) conditions (see Methods). To target MYC we used MYCi975, a small molecule that directly inhibits MYC activity, developed by Han et al. (Cancer Cell 36:483-497, 2019). To understand the dose-dependent effect of MYCi975 in Enzalutamide-naïve and Enzalutamide-resistant conditions, we performed dose-response assays in both LNCaP and C42B cell lines (FIG. 13A, FIG. 14A, see Methods) with varying doses of both drugs. This analysis demonstrated a striking reduction of the cell viability when treated with MYCi975 both in Enzalutamide-naïve and Enzalutamide-resistant conditions in a dose-dependent manner (FIG. 13A, FIG. 14A).

Next, we utilized identified sub-IC₅₀ concentrations of Enzalutamide (10 μM) alone, MYCi975 (2 μM) alone, or Enzalutamide and MYCi975 in combination, to perform colony formation assay using Enzalutamide-resistant LNCaP and C42B cells. Inhibition of MYC using MYCi975 reduced the colony formation of LNCaP-EnzaRes cells (one-tailed Welch t-test p-value=0.013, FIG. 14B) and C42B-EnzaRes cells (one-tailed Welch t-test p-value=3.98E-6, FIG. 13B), compared to Intact (DMSO). Interestingly, the colony formation ability was significantly reduced when MYCi975 and Enzalutamide were administered in combination on LNCaP-EnzaRes cells (one-tailed Welch t-test p-value=6.39E-5 FIG. 10B) and C42B-EnzaRes (one-tailed Welch t-test p-value=4.7E-8 FIG. 13B) compared to Intact (DMSO).

To evaluate the impact of Enzalutamide alone, MYCi975 alone, or in combination on the migratory capacity of Enzalutamide-resistant cells, we performed Boyden chamber-based in vitro migration assay using C42B-EnzaRes cells (LNCaP cells do not migrate) and observed a significant reduction in cell migration when treated with MYCi975 (one-tailed Welch t-test p-value=0.02, FIG. 13C) and even greater reduction when treated with a combination of MYCi975 and Enzalutamide (one-tailed Welch t-test p-value=0.003, FIG. 13C).

Next, to evaluate the effect of NME2 silencing on MYC expression, we first evaluated the expression of NME2 in EnzaRes compared to Intact in C42B cells, which showed elevated levels of NME2 in C42B-EnzaRes cells (FIG. 13D, one-tailed Welch t-test p-value=0.0005). NME2 knockdown in C42B-EnzaRes cells using two different siRNAs (FIG. 13E), demonstrated a significant reduction in expression of NME2 (FIG. 13E, left) and MYC (FIG. 13E, right), supported by the previously identified NME2 upstream regulation of MYC.

In addition, to evaluate if silencing of NME2 could enhance the effect of MYC inhibition on cells' metastatic potential, we performed Boyden chamber-based in vitro migration assay using C42B-EnzaRes cells, which demonstrated that the cell migration was further reduced when NME2 knockdown was added to MYCi975 administration (FIG. 13F).

Finally, C4-2B cells expressing doxycycline-inducible shRNA targeting NME2 were generated using the targeting sequence GAAATCAGCCTATGGTTTAAG (SEQ ID NO: 9). Cells were treated with 0-2000 ng/mL doxycycline for 96 hours and NME2 and MYC expression were evaluated by Western blot. This confirmed that NME2 knockdown suppresses MYC expression (FIG. 15).

Taken together, our results using relevant pre-clinical models of Enzalutamide resistance indicate that therapeutic targeting of MYC is beneficial in Enzalutamide-resistant conditions and MYC inhibition could be combined with concurrent Enzalutamide administration for improved efficacy. Further, apart from direct inhibition of MYC, its indirect inhibition via NME2 knockdown enhances MYC-related therapeutic targeting. We propose that MYC-centered therapeutic targeting could be an alternative therapy for patients at risk of Enzalutamide-resistance and/or salvage therapy for patients that failed Enzalutamide treatment.

In view of the many possible implementations to which the principles of the disclosure may be applied, it should be recognized that the illustrated implementations are only examples and should not be taken as limiting the scope of the invention. Rather, the scope of the invention is defined by the following claims. We therefore claim as our invention all that comes within the scope and spirit of these claims.

1. A method of treating a subject with prostate cancer, comprising:
(i) measuring expression of one or more enzalutamide resistance-related molecules from one or more enzalutamide resistance-related molecular pathways and/or transcriptional regulatory programs in a sample obtained from the subject, wherein the enzalutamide resistance-related molecular pathways or transcriptional regulatory programs comprise:
(a) a Myc molecular pathway; or
(b) a NME2 transcriptional regulatory program; or
(c) any combination thereof; and
(ii) administering enzalutamide to the subject with prostate cancer, wherein expression of the one or more enzalutamide resistance-related molecules is similar to a control representing expression for the one or more enzalutamide resistance-related molecules expected in a sample from a subject who positively responds to enzalutamide therapy; or
administering an androgen receptor signaling inhibitor that is not enzalutamide to the subject with prostate cancer, wherein expression of the one or more enzalutamide resistance-related molecules differs from a control representing expression for the one or more enzalutamide resistance-related molecules expected in a sample from a subject who positively responds to enzalutamide therapy,
thereby treating the subject with prostate cancer.

2. The method of claim 1, wherein the androgen receptor signaling inhibitor that is not enzalutamide is abiraterone.

3. A method of identifying a subject with prostate cancer who will respond positively to enzalutamide therapy, comprising:
measuring expression of one or more enzalutamide resistance-related molecules from one or more enzalutamide resistance-related molecular pathways or transcriptional regulatory programs in a sample obtained from the subject, wherein the enzalutamide resistance-related molecular pathways or transcriptional regulatory programs comprise:
(a) a Myc molecular pathway;
(b) a NME2 transcriptional regulatory program; or
(c) any combination thereof,
wherein expression of the enzalutamide resistance-related molecules is similar to a control representing expression for the enzalutamide resistance-related molecules expected in a sample from a subject who positively responds to enzalutamide therapy,
thereby identifying a subject with prostate cancer who will respond positively to enzalutamide therapy.

4. The method of claim 1, wherein the one or more enzalutamide resistance-related molecules from one or more enzalutamide resistance-related molecular pathways or transcriptional regulatory programs comprise:
(a) one or more of MYC, SLC19A1, MRTO4, TMEM97, RRP9, PES1, TFB2M, EXOSC5, IPO4, NDUFAF4, NOC4L, SRM, PA2G4, GNL3, NOLC1, WDR43, RABEPK, NOP16, TBRG4, DDX18, NIP7, WDR74, BYSL, HSPD1, PLK4, NOP2, PPAN, NOP56, RCL1, NPM1, AIMP2, RRP12, PPRC1, TCOF1, MCM5, HK2, CBX3, PLK1, PHB, MCM4, CDK4, DUSP2, MYBBP1A, UTP20, PRMT3, FARSA, MAP3K6, LAS1L, PUS1, HSPE1, SLC29A2, DCTPP1, SUPV3L1, SORD, IMP4, GRWD1, UNG, and MPHOSPH10 from the Myc molecular pathway;
(b) NME2; or
(c) any combination thereof.

5. The method of claim 1, wherein expression of the one or more enzalutamide resistance-related molecules from one or more enzalutamide resistance-related molecular pathways or transcriptional regulatory programs is measured by RNAseq or qRT-PCR.

6. The method of claim 5, wherein the one or more enzalutamide resistance-related molecules from one or more enzalutamide resistance-related molecular pathways or transcriptional regulatory programs comprise Myc, NME2, or both.

7. The method of claim 1, wherein the subject who positively responds to enzalutamide therapy is a subject who does not develop resistance to enzalutamide therapy.

8. The method of claim 1, wherein treating the subject with enzalutamide therapy occurs wherein the subject is identified as a subject who responds positively to enzalutamide therapy with a p value of 0.01 or less.

9. The method of claim 3, wherein the subject is identified as a subject who will respond positively to enzalutamide therapy with a p value of 0.01 or less.

10. The method of claim 8, wherein the p value is 0.0035.

11. The method of claim 1, wherein the expression comprises mRNA expression.

12. The method of claim 1, wherein the sample is a prostate cancer sample.

13. The method of claim 1, wherein the subject is human.

14. The method of claim 3, wherein a subject that responds positively to enzalutamide therapy is a subject:
with a prostate cancer that is reduced in size by at least 20%, at least 50%, at least 80%, at least 90%, at least 95%, at least 98%, or even at least 100%, following administration of the enzalutamide therapy, as compared to no treatment with the enzalutamide therapy;
with a metastasis that is reduced in size by at least 20%, at least 50%, at least 80%, at least 90%, at least 95%, at least 98%, or even at least 100%, following administration of the enzalutamide therapy, as compared to no treatment with the enzalutamide therapy;
has an increase in survival time following administration of the enzalutamide therapy, as compared to no treatment with the enzalutamide therapy;
has a reduction of at least 65%, at least 85%, at least 90%, at least 95%, or at least 98%, in developing resistance to the enzalutamide therapy; or
combinations thereof,
within one year of starting treatment with the enzalutamide therapy.

15. The method of claim 1, further comprising administering subject an inhibitor of a Myc molecular pathway or a NME2 transcriptional regulatory program to the subject.

16. A method of treating a subject with prostate cancer, comprising administering to the subject an inhibitor of a Myc molecular pathway or a NME2 transcriptional regulatory program,
wherein the subject has an enzalutamide-resistant prostate cancer that expresses increased Myc and NME2 compared to a sample from a subject who positively responds to enzalutamide therapy,
thereby treating the prostate cancer.

17. The method of claim 16, wherein the inhibitor of the Myc molecular pathway inhibits Myc expression or activity.

18. The method of claim 17, wherein the inhibitor of the Myc molecular pathway is Myc-i975.

19. The method of claim 16, wherein the inhibitor of the NME2 transcriptional regulatory program inhibits NME2 expression or activity.

20. The method of claim 19, wherein the inhibitor of the NME2 transcriptional regulatory program is an antisense oligonucleotide or siRNA that specifically binds to an NME2 nucleic acid.

21. The method of claim 16, further comprising treating the subject with enzalutamide.

22. The method of claim 16, wherein the subject with prostate cancer is a subject that has received enzalutamide therapy and undergone disease progression.
